(19) United States
(12) Patent Application Publication
(10) Pub. No.: US 2022/0008414 A1
(43) Pub. Date: Jan. 13, 2022

(54) PHARMACEUTICAL COMPOSITION COMPRISING HISTONE DEACETYLASE 6 INHIBITORS

(71) Applicant: Chong Kun Dang Pharmaceutical Corp., Seoul (KR)

(72) Inventors: Young II Choi, Gyeonggi-do (KR); Nina Ha, Gyeonggi-do (KR); Ju Young Song, Gyeonggi-do (KR); Min Cheol Kim, Gyeonggi-do (KR); Daekwon Bae, Gyeonggi-do (KR); Ji Yeon Baek, Gyeonggi-do (KR)

(21) Appl. No.: 17/296,008

(22) PCT Filed: Nov. 22, 2019
(86) PCT No.: PCT/KR2019/016202

§ 371 Date: May 21, 2021

(30) Foreign Application Priority Data
Nov. 23, 2018 (KR) 10-2018-0146732

(51) Int. Cl. A61K 31/496; A61P 25/02; A61K 31/495

(52) U.S. Cl. CPC A61K 31/496; A61K 31/495; A61P 25/02

(57) ABSTRACT

The present invention relates to a pharmaceutical composition for preventing or treating CMT disease, comprising histone deacetylase 6 inhibitor. The pharmaceutical composition according to the present invention has histone deacetylase 6 (HDAC6) inhibitory activity and thus is effective in preventing or treating CMT disease.

### TECHNICAL FIELD

The present invention relates to a pharmaceutical composition for preventing or treating CMT disease, comprising histone deacetylase 6 (HDAC6) inhibitor as an active ingredient; a method for treating using the histone deacetylase 6 (HDAC6) inhibitor; and a use of histone deacetylase 6 (HDAC6) inhibitor in preparing a drug for preventing or treating CMT disease.

### BACKGROUND ART

Charcot-Marie-Tooth (CMT, hereditary motor and sensory neuropathy: HMSN) disease is the most common type of hereditary peripheral nerve disorder caused by a mutation of proteins which constitute nerves. More than 1,000 mutations have been identified from about 90 genes so far (Timmerman et al., (2014) Genes 5:13-32). Upon the development of Charcot-Marie-Tooth (CMT) disease, a progressive degeneration of peripheral nerves leads to atrophy of muscles affected by neural distribution, such that patients show a gradual atrophy in their muscles of hands and feet as well as symptoms of deformed hands and feet. CMT is genetically and clinically very diverse and complicated, and it is known that symptoms thereof vary ranging from a close-to-normal state to a wheelchair-bound state depending on mutation types. CMT emerges mainly in teen years and occurs to one for every 2,500 people (Krajewski et al., (2000) Brain 123:1516).

CMT belongs to rare diseases as a hereditary peripheral nerve disorder. However, a prevalence rate thereof amounts to 1 per 2,500 of the population. There are about 20,000 patients in South Korea and 2,800,000 ones worldwide. Until now, treatment for CMT is limited only to rehabilitation, aids, pain control, surgical therapy, etc., but a successful therapeutic agent has not been developed yet. Thus, there is a very urgent need for developing a therapeutic agent for CMT.

For example, with regard to CMT, i.e., the most common type out of hereditary motor and sensory neuropathies, a large-scale clinical trial was conducted on ascorbic acid, which had been proven as an essential material for myelination in the peripheral nervous system through an experiment on culturing lemmocytes and dorsal root ganglion cells together, but such trial was failed in proving validity (Pareyson et al., (2011) 10(4):3205).

### PRIOR ART REFERENCES

Korean Patent Registration No. 1697518

Krajewski et al., (2000) Brain 123:1516

Pareyson et al., (2011) 10(4):3205

### DISCLOSURE OF INVENTION

### Technical Problem

An objective of the present invention is to provide a pharmaceutical composition for preventing or treating Charcot-Marie-Tooth (CMT) disease, comprising the compound of the present invention, isomers thereof or pharmaceutically acceptable salts thereof.

Other objective of the present invention is to provide a use of the compound of the present invention, isomers thereof or pharmaceutically acceptable salts thereof in preparation of a medicament for preventing or treating Charcot-Marie-Tooth (CMT) disease.

Another objective of the present invention is to provide a method for preventing or treating Charcot-Marie-Tooth (CMT) disease, comprising a step of administering a therapeutically effective amount of the compound of the present invention, isomers thereof or pharmaceutically acceptable salts thereof into a subject.

Yet another objective of the present invention is to provide a use of the compound of the present invention, isomers thereof or pharmaceutically acceptable salts thereof for preventing or treating Charcot-Marie-Tooth (CMT) diseases.

### Solution to Problem

This is described in detail as follows. Meanwhile, each description and embodiment disclosed in the present invention may be also applied to other descriptions and embodiments thereof, respectively. In other words, all the combinations of various elements disclosed in the present invention fall within the scope of the present invention. Also, it cannot be seen that the scope of the present invention is limited to the specific description described below.

The present invention provides a pharmaceutical composition for preventing or treating Charcot-Marie-Tooth (CMT) disease, comprising histone deacetylase 6 inhibitor.

In the present invention, the histone deacetylase 6 inhibitor may be a compound represented by the following Formula I, isomers thereof or pharmaceutically acceptable salts thereof:

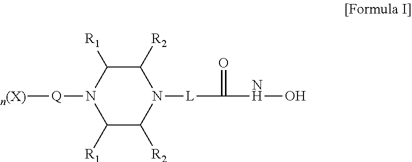

wherein

R₁ is hydrogen or —CH₃,

R₂ is hydrogen or —CH₃, wherein R₂ is —CH₃ when R₁ is hydrogen, and R₂ is hydrogen when R₁ is —CH₃,

L is —(C₄-C₅ alkyl)-; —(C₁-C₃ alkyl)-L₁-; —C(═O)-L₁- or —S(═O)₂-L₁-,

wherein —(C₄-C₅ alkyl)- and —(C₁-C₃ alkyl)- may be unsubstituted or substituted with —CH₃

L₁ is —(C₃-C₆)cycloalkyl-;

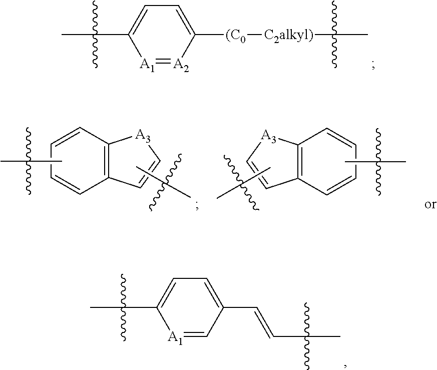

A₁ and A₂ are each independently —N— or —CR₃—, wherein both A₁ and A₂ cannot be —N—,

R₃ is hydrogen; —F, —Cl, —Br, —I or —OH, and

A₃ is —NH— or —O—,

Q is selected from the group consisting of —(C₁-C₆)alkyl-; —(C₂-C₆)alkenyl-; —C(═O)—; —C(═S)—; —S(═O)₂— and Q

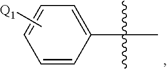

wherein —(C₁-C₆)alkyl- and —(C₂-C₆)alkenyl- may be unsubstituted or each independently substituted 1 to 3 —CH₃ groups or halogen atoms,

Q₁ is hydrogen; —F, —Cl, —Br or —I,

n is an integer of 0, 1 or 2, wherein n is 0 when Q is

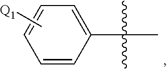

n is 1 when Q is —C(═O)—; —C(═S)— or —S(═O)₂—, and n is 1 or 2 when Q is —(C₁-C₆)alkyl- or —(C₂-C₆)alkenyl-, and

X may be selected from the group consisting of —C₁-C₆ alkyl; —C₃-C₆ cycloalkyl; —C₂—C₆ alkenyl; —C₃-C₆ cycloalkenyl; —(C₀-C₂ alkyl)Ar; —OAr; —(C₀-C₂ alkyl)Het; naphthyl and following groups:

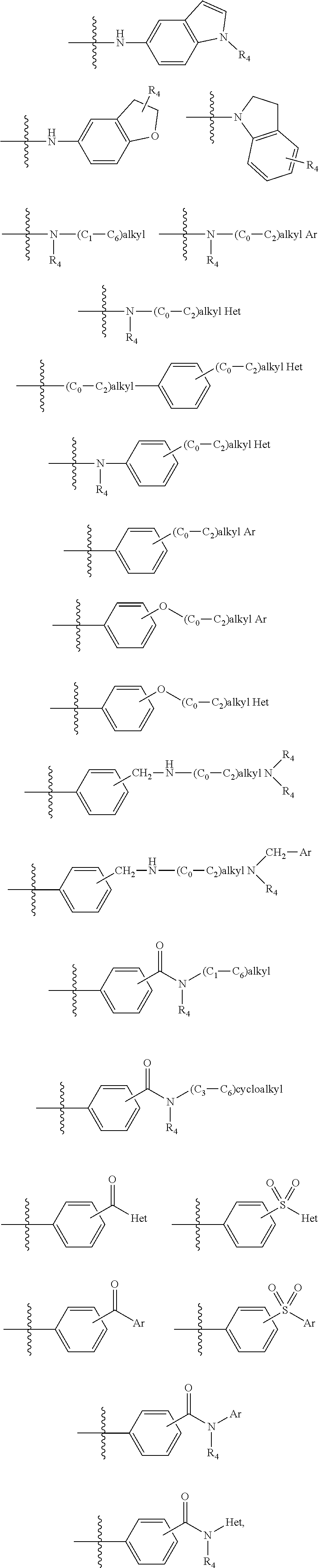

wherein R₄ is H or —C₁-C₄ alkyl,

—C₀-C₂ alkyl, —C₂-C₆ alkenyl and —C₁-C₆ alkyl may be unsubstituted or substituted with 1 to 2 —CH₃ groups; 1 to 3 —F groups; or both,

Ar is a C₆ monocyclic aromatic compound, which may be unsubstituted or substituted with one or more halogen atoms; —OH; —NH₂; —C₁-C₆ alkyl; —O(C₁-C₆)alkyl; —C₃-C₆ cycloalkenyl; —NH(C₁-C₆ alkyl); —N(C₁-C₃ alkyl)₂; —CH₂N(C₁-C₃ alkyl)₂; —S(═O)₂—(C₁-C₃ alkyl) or phenyl groups, wherein —C₁-C₃ alkyl; —C₁-C₆ alkyl and —C₃-C₆ cycloalkenyl may be each independently substituted with 1 to 5 —F or —CH₃ groups, and

Het is a 4- to 6-membered heteroaromatic or non-aromatic ring compound containing 1 to 3 elements selected from the group consisting of N, O and S while having 0 to 3 double bonds, and may be unsubstituted or substituted with one or more halogen atoms; —C₁-C₆ alkyl; —C(═O)(C₁-C₃ alkyl); —S(═O)₂ (C₁-C₃ alkyl) or benzyl groups, wherein —C₁-C₃ alkyl and —C₁-C₆ alkyl may be each independently substituted with —OH; 1 to 5 —F or —CH₃ groups.

As used herein, “C₀ alkyl” means that there is no carbon and therefore represents a bond. For example, “—(C₀ alkyl)Ar” means —Ar.

The compounds represented by formula I according to the present invention may be compounds represented by the following formula II or formula III:

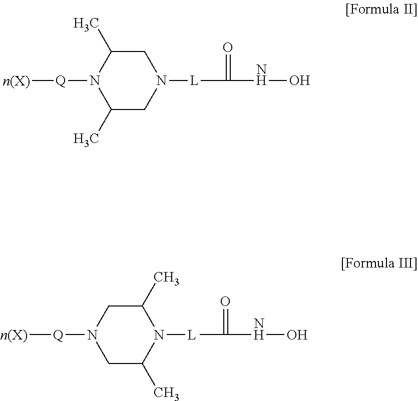

wherein

L is —(C₅ alkyl)-; —(C₁-C₂ alkyl)-L₁-; —C(═O)-L₁- or —S(═O)₂-L₁-,

wherein —(C₅ alkyl)- and —(C₁-C₂ alkyl)- are straight-chain and may be unsubstituted or substituted with —CH₃,

L₁ is —(C₃-C₆) cycloalkyl-;

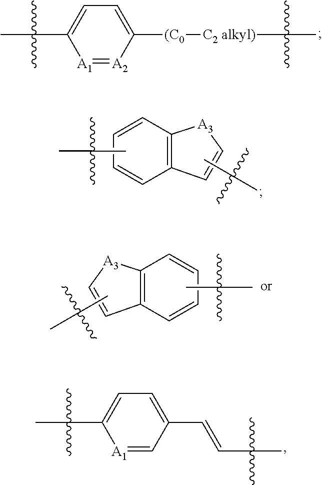

A₁ and A₂ are each independently —N— or —CR₃—, wherein both A₁ and A₂ cannot be —N—,

R₃ is hydrogen; —F or —OH, and

A₃ is —NH— or —O—,

Q is selected from the group consisting of —(C₁-C₃)alkyl-; —C(═O)—; —C(═S)—; —S(═O)₂- and

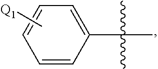

wherein —(C₁-C₃)alkyl- may be unsubstituted or substituted with 1 to 3 —CH groups or halogen atoms,

Q₁ is hydrogen; —F or —Cl,

n is an integer of 0 or 1, wherein n is 0 when Q is

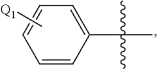

and n is 1 when Q is —C(═O)—, —C(═S)—, —S(═O)₂— or —(C₁-C₃)alkyl-, and

X may be selected from the group consisting of —C₁-C₆ alkyl; —C₃-C₆ cycloalkyl; —C₂—C₆ alkenyl; —C₃-C₆ cycloalkenyl; —(C₀-C₂ alkyl)Ar; —OAr; —(C₀-C₂ alkyl)Het; naphthyl; and following groups:

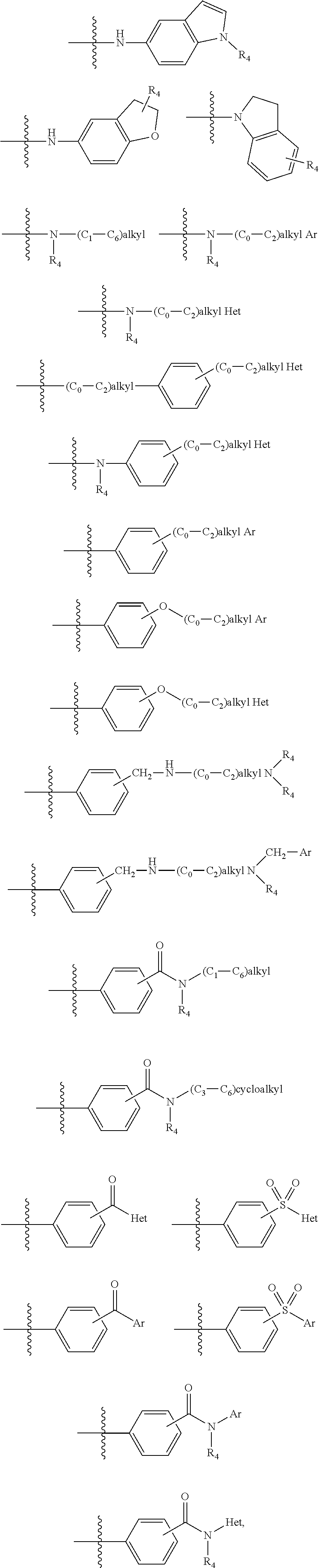

wherein R₄ is H or —C₁-C₄ alkyl,

—C₀-C₂ alkyl; —C₂-C₆ alkenyl and —C₁-C₆ alkyl may be unsubstituted or substituted with 1 or 2 —CH₃ groups or 1 to 3 —F groups,

Ar is a C₆ monocyclic aromatic compound, which may be unsubstituted or substituted with one or more halogen atoms; —OH; —NH₂; —C₁-C₆ alkyl; —O(C₁-C₆)alkyl; —C₃-C₆ cycloalkenyl; —NH(C₁-C₆ alkyl); —N(C₁-C₃ alkyl)₂; —CH₂N(C₁-C₃ alkyl)₂; —S(═O)₂—(C₁-C₃ alkyl) or phenyl groups, wherein —C₁-C₃ alkyl; —C₁-C₆ alkyl and —C₃ —C₆ cycloalkenyl may be each independently substituted with 1 to 5 —F or —CH₃ groups, and

Het is a 4- to 6-membered heteroaromatic or non-aromatic ring compound containing 1 to 3 elements selected from the group consisting of N; O and S while having 0 to 3 double bonds, and may be unsubstituted or substituted with one or more halogen atoms; —C₁-C₆ alkyl; —C(═O)(C₁-C₃ alkyl); —S(═O)₂ (C₁-C₃ alkyl) or benzyl groups, wherein —C₁-C₃ alkyl and —C₁-C₆ alkyl may be each independently substituted with —OH; or 1 to 5 —F or —CH₃ groups.

In a preferred embodiment of the present invention, L, Q and X in Formulas I, II and III may be defined as follows:

L is —CH₂-L₁-,

wherein

L₁ is

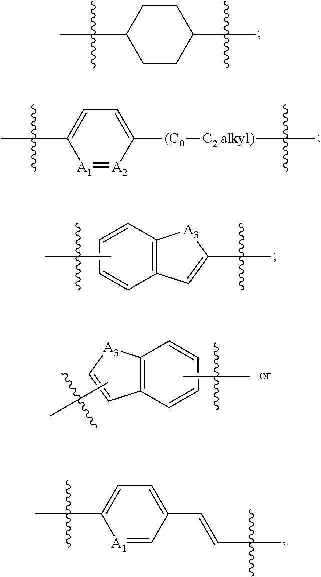

A₁ and A₂ are each independently —N— or —CR₃—, wherein both A₁ and A₂ cannot be —N—,

R₃ is hydrogen; —F or —OH, and

A₃ is —NH— or —O—,

Q is —CH₂-, —C(═O)— or —S(═O)₂-, and

X may be selected from the group consisting of —C₁-C₆ alkyl; —(C₀-C₂ alkyl)Ar; —(C₀ —C₂ alkyl)Het; —OAr; and following groups:

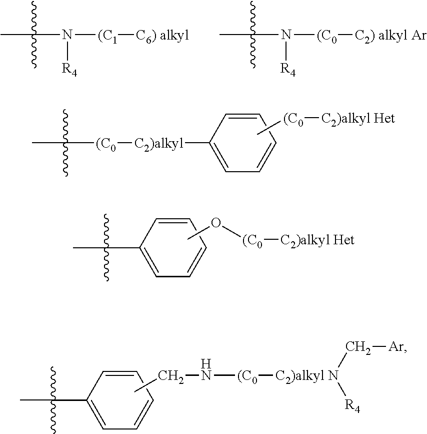

wherein R₄ is H or —C₁-C₄ alkyl,

—C₀-C₂ alkyl and —C₁-C₆ alkyl may be unsubstituted or substituted with 1 or 2 —CH₃ groups and/or 1 to 3 —F groups, and

Ar and Het is each independently as defined in formula I to formula III.

In an embodiment of the present invention, the heterocyclic compound (Het) mentioned as X or a substituent in formula I to formula III above may have a structure selected from the following group:

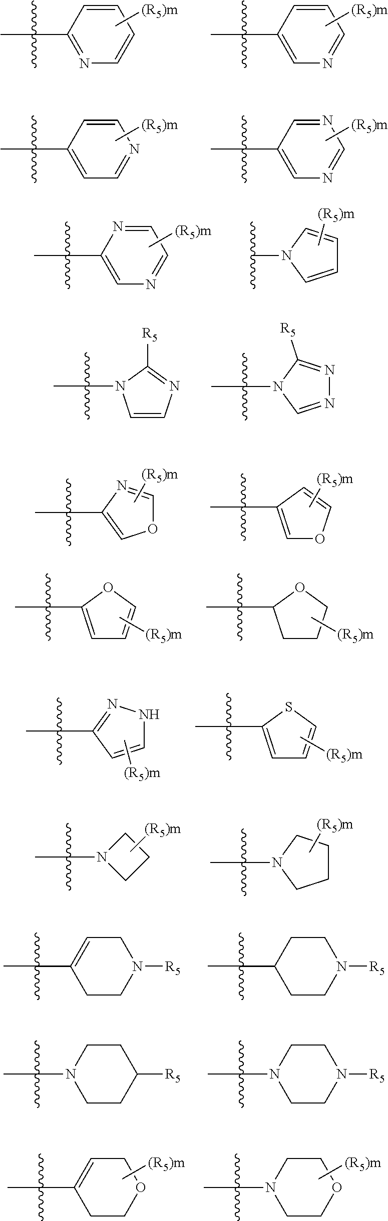

wherein

R₅ are each independently hydrogen; —F; —Cl; —C₁-C₆ alkyl; —C(═O)(C₁-C₃ alkyl); —S(═O)₂ (C₁-C₃ alkyl) or benzyl, wherein —C₁-C₃ alkyl and —C₁-C₆ alkyl may be each independently substituted with —OH; 1 to 5 —F or —CH₃ groups,

m is an integer of 0, 1, 2 or 3, and

Het is unsubstituted when m is 0, and Het may be substituted with independent R₅ when m is 1, 2 or 3.

The compounds represented by formula I according to the present invention may be compounds represented by the following formula I-1:

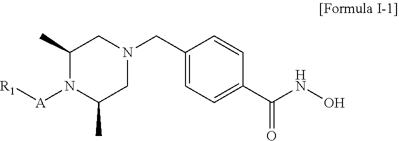

wherein

A is —C(═O)—; —CH₂—; —NH(C═O)—; —C(═S)— or —S(═O)₂—, and

R₁ is a straight or branched chain C₁-C₄ alkyl; a straight or branched chain C₁-C₄ alkenyl; a C₃-C₅ cycloalkyl; —OC₆C₅; —(C₀-C₂ alkyl)CF₃; phenyl (which may be unsubstituted or substituted with C₁-C₃ alkyl; pyrrole; —OCH₃; —CF₃; —F; —Cl or —OH); pyridyl; furanyl; thiophenyl; benzyl (which may be unsubstituted or substituted with one or more C₁-C₃ alkyl; pyrrole; —OCH₃; —CF₃; —F; —Cl or —OH groups); phenethyl; naphthyl; oxazolyl; pyrimidinyl; pyrazolyl or pyrazinyl.

The compounds represented by formula I-1 are preferably compounds 82, 83, 84, 98, 99, 100, 120, 121, 122, 123, 125, 126, 127, 128, 145, 146, 147, 148, 149, 159, 160, 161, 177, 184, 188, 204, 211, 212, 213, 214, 222, 223, 224, 225, 232, 255, 265, 266, 267, 270, 272, 275, 290, 291, 292, 293, 294, 295, 296, 297, 354, 355, 403, 404 and 405 as disclosed herein.

The compounds represented by formula I may be compounds represented by the following formula I-2:

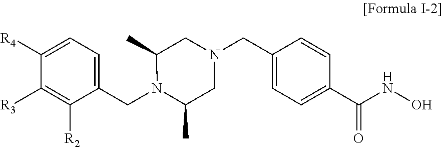

wherein R₂, R₃ or R₄ may be each independently hydrogen or any one selected from the following group:

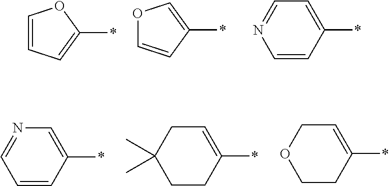

The compounds represented by formula I-2 are preferably compounds 309, 327, 328, 329, 330, 331, 332, 342, 343, 344, 345, 346 and 347 as disclosed herein.

The compounds represented by formula I according to the present invention may be compounds represented by the following formula I-3:

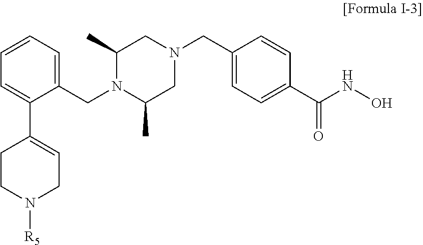

wherein R₅ may be selected from the group consisting of a straight or branched chain C₁-C₄ alkyl; —COCH₃; —CH₂CF₃ and —SO₂CH₃.

The compounds represented by formula I-3 are preferably compounds 481, 482, 483, 484 and 485 as disclosed herein.

The compounds represented by formula I according to the present invention may be compounds represented by formula I-4:

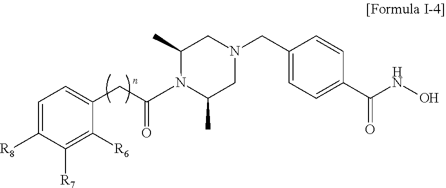

wherein n is an integer of 0, 1 or 2, and R₆, R₇ or R₈ may be each independently hydrogen or any one selected from the group consisting of the following group:

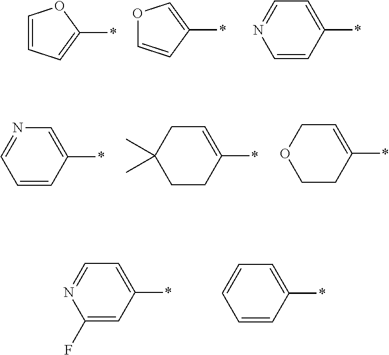

The compounds represented by formula I-4 are preferably compounds 234, 242, 243, 244, 245, 246, 247, 283, 284, 285, 286, 288, 326 and 340 as disclosed herein.

The compounds represented by formula I according to the present invention may be compounds represented by formula I-5:

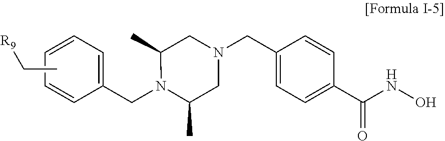

wherein R₉ may be selected from among morpholine; piperidine; pyrrolidine; azetidine; piperazine; —C₁-C₂ primary or secondary alkylamine;

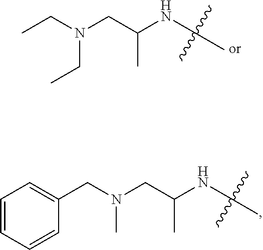

wherein pyrrolidine may be unsubstituted or substituted with one or more —F or —Cl groups, azeditine may be unsubstituted or substituted with one or more —F or —Cl groups, and piperazine may be unsubstituted or substituted with one or more straight or branched chain C₁-C₅ alkyl; benzyl; —COCH₃; —CH₂CF₃ or —SO₂CH₃ groups.

The compounds represented by formula I-5 are preferably compounds 356, 376, 382, 383, 384, 385, 411, 412, 413, 426, 427, 428, 429, 430, 431, 452, 453, 454, 455, 456, 457, 466, 467, 468 and 486 as disclosed herein.

The compounds represented by formula I according to the present invention may be compounds represented by the following formula I-6_{A} or formula I-6_{B}:

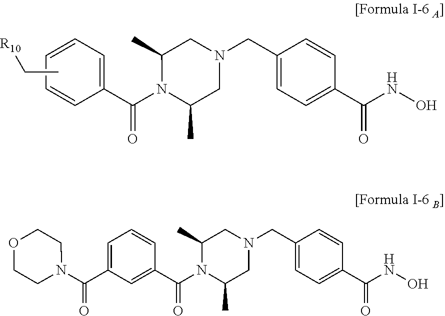

wherein R₁₀ may be selected from among pyrrolidine; piperidine; C₁-C₂ primary or secondary alkylamine; piperazine; morpholine;

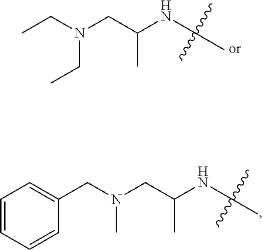

wherein pyrrolidine may be unsubstituted or substituted with one or more —F or —Cl groups, and piperazine may be unsubstituted or substituted with one or more straight or branched chain C₁-C₅ alkyl; —COCH₃; —CH₂CF₃ or —SO₂CH₃ groups.

The compounds represented by formula I-6_{A} are preferably compounds 423, 424, 425, 432, 433, 434, 435, 439, 440, 441, 442, 443, 444, 458, 459, 460, 461, 462 and 463 as disclosed herein. Furthermore, the compounds represented by formula I-6_{B} are preferably compound 446 as disclosed herein.

The compounds represented by formula I according to the present invention may be compounds represented by the following formula I-7:

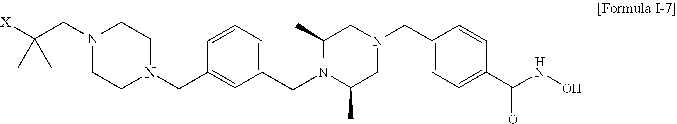

wherein X may be selected from —OH; —F; —Cl or —Br.

The compounds represented by formula I-7 are preferably compounds 386 and 387 as disclosed herein.

The compounds represented by formula I according to the present invention may be compounds represented by the following formula I-8:

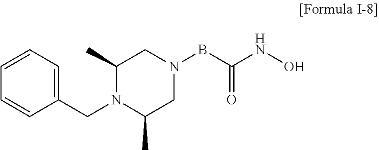

wherein B may be selected from among

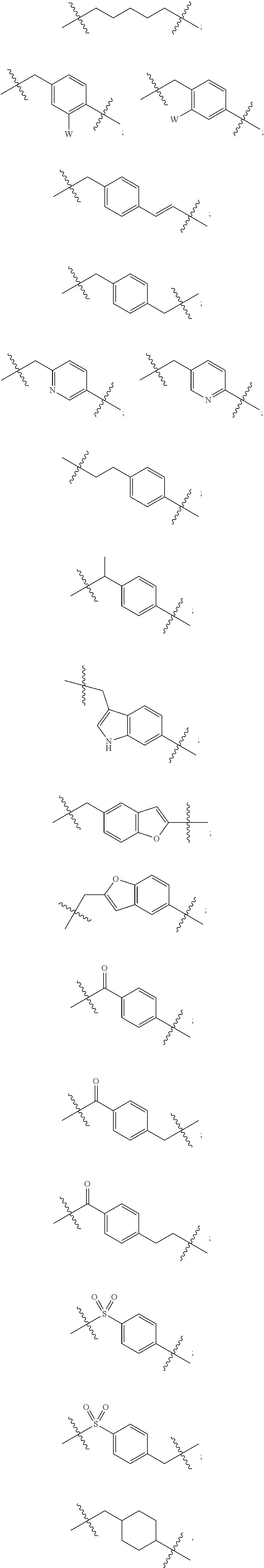

wherein w is hydrogen; —F; —Cl or —OH.

The compounds represented by formula I-8 are preferably compounds 154, 171, 172, 173, 194, 218, 219, 520, 571, 574, 652, 812, 813, 814, 818, 820, 822, 823 and 824 as disclosed herein.

The compounds represented by formula I according to the present invention may be compounds represented by the following formula I-9:

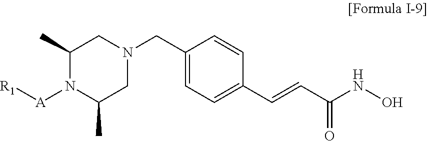

wherein A is —C(═O)—; —CH₂-; —NH(C═O)—; —C(═S)— or —S(═O)₂-, and R₁₁ may be selected from among furanyl; benzyl; pyrrole-substituted phenyl or pyrrole-substituted anilinyl.

The compounds represented by formula I-9 are preferably compounds 321, 322 and 323 as disclosed herein.

The compounds represented by formula I according to the present invention may be compounds represented by the following formula I-10:

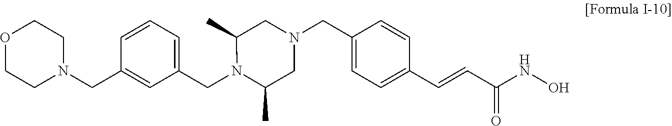 [SMILES: C[C@@H]1CN(Cc2ccc(/C=C/C(=O)NO)cc2)C[C@H](C)N1Cc1cccc(CN2CCOCC2)c1]

The compounds represented by formula I-10 are preferably compound 472 as disclosed herein.

The compounds represented by formula I according to the present invention may be compounds represented by the following formula I-11:

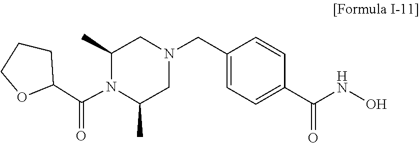 [SMILES: C[C@@H]1CN(Cc2ccc(C(=O)NO)cc2)C[C@H](C)N1C(=O)C1CCCO1]

The compounds represented by formula I-11 are preferably compound 402 as disclosed herein.

The compounds represented by formula I according to the present invention may be compounds represented by the following formula I-12:

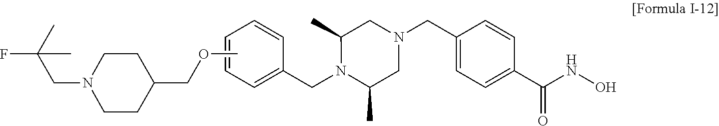

The compounds represented by formula I-12 are preferably compounds 380 and 388 as disclosed herein.

The compounds represented by formula I according to the present invention may be compounds represented by the following formula I-13:

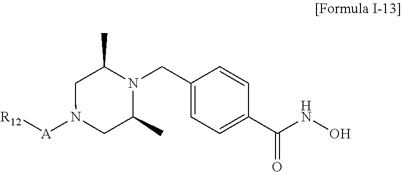

wherein A is —C(═O)—; —CH₂—; —CH(CH₃)—; —NH(C═O)—; —NCH₃(C═O)—; —C(═S)— or —S(═O)₂—, and R₁₂ may be selected from among straight or branched C₁-C₄ alkyl; phenyl (which may be unsubstituted or substituted with one or more straight or branched chain C₁-C₃ alkyl; —F; —Cl; CF₃; —OCH₃; —C₁-C₂ primary or secondary alkylamine; —SO₂CH₃; thiophenyl; pyrrole; pyrazolyl; furanyl; triazolyl or im-idazolyl); pyridinyl; thiophenyl; furanyl; benzyl (which may be unsubstituted or substituted with —F, —Cl or —OCH₃); indole (which may be unsubstituted or substituted with one or more straight or branched chain C₁-C₃ alkyl groups); dihydroben-zofuranyl; phenylamine (which may be unsubstituted or substituted with straight or branched chain C₁-C₃ alkyl); indoline or naphthyl.

The compounds represented by formula I-13 are preferably compounds 80, 81, 103, 104, 105, 106, 107, 108, 109, 110, 111, 112, 113, 114, 115, 118, 162, 163, 164, 165, 166, 167, 168, 183, 185, 186, 187, 189, 196, 197, 215, 220, 230, 231, 233, 256, 268, 271, 273, 274, 298, 299, 300, 301, 302, 303, 304 and 305 as disclosed herein.

The compounds represented by formula I according to the present invention may be compounds represented by the following formula I-14:

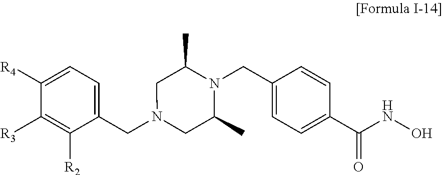

wherein R₂, R₃ and R₄ may be each independently hydrogen or any one selected from the following group:

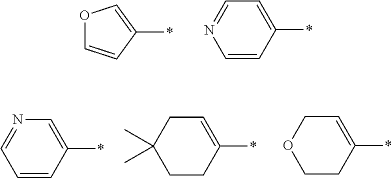

The compounds represented by formula I-14 are preferably compounds 348, 349, 350, 351, 352, 396, 400 and 401 as disclosed herein.

The compounds represented by formula I according to the present invention may be compounds represented by the following formula I-15:

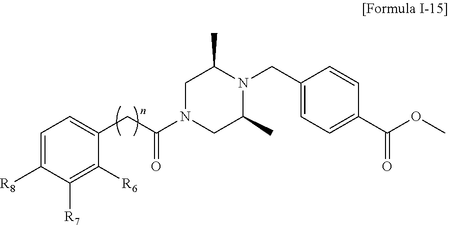

wherein n is an integer of 0, 1 or 2, and R₆, R₇ and R₈ may be each independently hydrogen or any one selected from the following group:

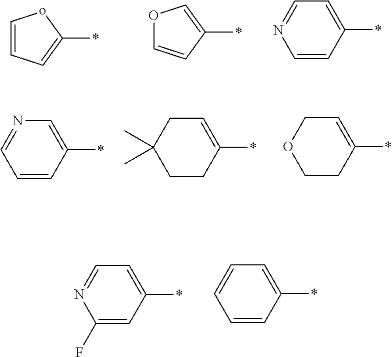

The compounds represented by formula I-15 are preferably compounds 250, 251, 252, 253, 257, 258, 259, 260, 261, 262, 263, 276, 277, 278, 279 and 280 as disclosed herein.

The compounds represented by formula I according to the present invention may be compounds represented by the following formula I-16:

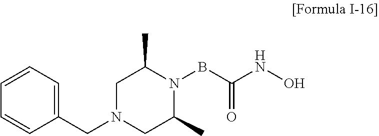

wherein B may be selected from among

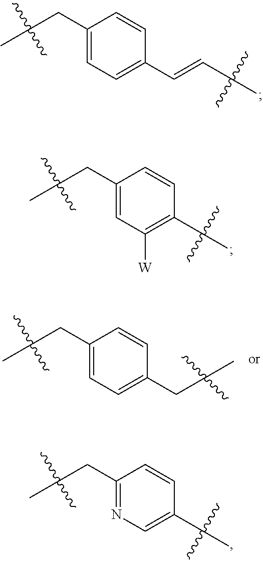

wherein w may be substituted with —F or —Cl.

The compounds represented by formula I-16 are preferably compounds 174, 175, 176 and 195 as disclosed herein.

The compounds represented by formula I according to the present invention may be compounds represented by the following formula I-17:

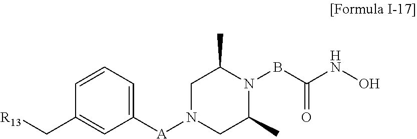

wherein A is —C(═O)—; —CH₂—; —NH(C═O)—; —C(═S)— or —S(═O)₂—, and B may be selected from among

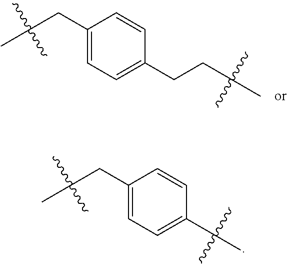

Also, R₁₃ may be or selected from among pyrrolidine and —C₁-C₂ primary or secondary alkylamine.

The compounds represented by formula I-17 are preferably compounds 475, 476, 478, 479, 480 and 487 as disclosed herein.

The compounds represented by formula I according to the present invention may be compounds represented by the following formula I-18:

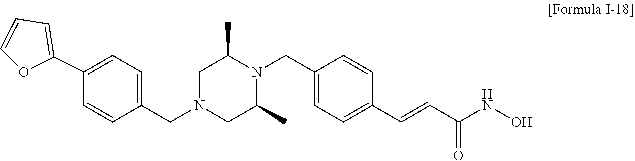 [SMILES: C[C@@H]1CN(Cc2ccc(-c3ccco3)cc2)C[C@H](C)N1Cc1ccc(/C=C/C(=O)NO)cc1]

The compounds represented by formula I-18 are preferably compound 477 as disclosed herein.

The compounds represented by formula I according to the present invention may be compounds represented by the following formula I-19:

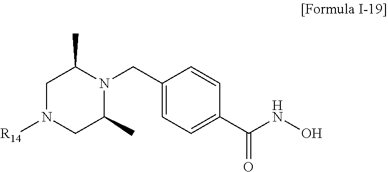

wherein R₁₄ may be selected from among

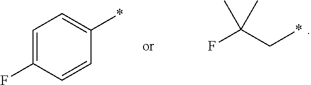

The compounds represented by formula I-19 are preferably compounds 119 and 193 as disclosed herein.

The compounds represented by formula I according to the present invention may be compounds represented by the following formula I-20:

[Formula I-20]

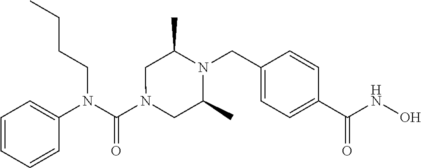 [SMILES: CCCCN(C(=O)N1C[C@@H](C)N(Cc2ccc(C(=O)NO)cc2)[C@@H](C)C1)c1ccccc1]

The compounds represented by formula I-20 are preferably compound 198 as disclosed herein.

The compounds represented by formula I according to the present invention may be compounds represented by the following formula I-21:

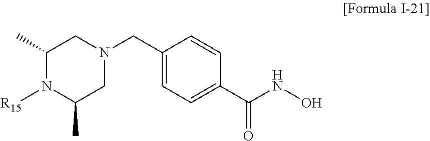

wherein R₁₅ may be selected from among

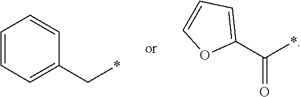

The compounds represented by formula I-21 are preferably compounds 248 and 249 as disclosed herein.

The compounds represented by formula I according to the present invention may be compounds represented by the following formula I-22:

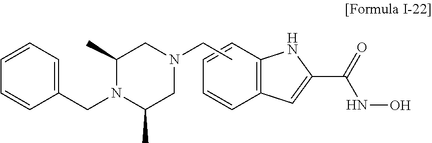

The compounds represented by formula I-22 are preferably compounds 569 and 573 as disclosed herein.

The compounds represented by formula I according to the present invention may be compounds represented by the following formula I-23:

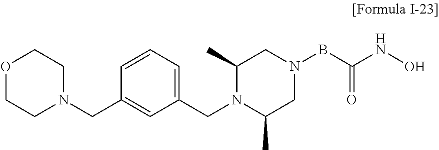

wherein B may be selected from among

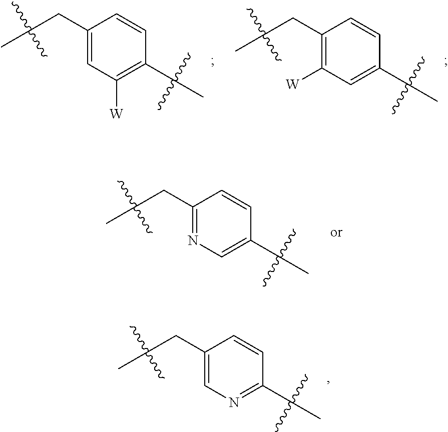

wherein w is hydrogen; —F; —Cl or —OH.

The compounds represented by formula I-23 are preferably compounds 609, 653 and 696 as disclosed herein.

The compounds represented by formula I, formula II, formula III and formulas I-1 to I-23 are shown below.

| No. | Name of Compound |
|---|---|
| 080 | 4-(((2S,6R)-4-benzoyl-2,6-dimethylpiperazin-1- |
| | yl)methyl)-N-hydroxybenzamide |
| 081 | 4-(((2S,6R)-4-benzyl-2,6-dimethylpiperazin-1- |
| | yl)methyl)-N-hydroxybenzamide |
| 082 | 4-(((3R,5S)-4-acetyl-3,5-dimethylpiperazin-1- |
| | yl)methyl)-N-hydroxybenzamide |
| 083 | 4-(((3R,5S)-4-benzoyl-3,5-dimethylpiperazin-1- |
| | yl)methyl)-N-hydroxybenzamide |
| 084 | 4-(((3R,5S)-4-benzyl-3,5-dimethylpiperazin-1- |
| | yl)methyl)-N-hydroxybenzamide |
| 098 | (2S,6R)-4-(4-(hydroxycarbamoyl)benzyl)-N-isopropyl- |
| | 2,6-dimethylpiperazine-1-carboxamide |
| 099 | (2S,6R)-N-(3-chlorophenyl)-4-(4-(hydroxycarbamoyl) |
| | benzyl)-2,6-dimethylpiperazine-1-carboxamide |
| 100 | 4-(((3R,5S)-3,5-dimethyl-4-(phenylsulfonyl)piperazin- |
| | 1-yl)methyl)-N-hydroxybenzamide |
| 103 | (3R,5S)-4-(4-(hydroxycarbamoyl)benzyl)-N-isopropyl-3,5- |
| | dimethylpiperazine-1-carboxamide |
| 104 | (3R,5S)-N-(3-chlorophenyl)-4-(4-(hydroxycarbamoyl) |
| | benzyl)-3,5-dimethylpiperazine-1-carboxamide |
| 105 | 4-(((2S,6R)-2,6-dimethyl-4-(phenylsulfonyl)piperazin- |
| | 1-yl)methyl)-N-hydroxybenzamide |
| 106 | 4-(((2S,6R)-4-(4-chlorobenzoyl)-2,6-dimethylpiperazin- |
| | 1-yl)methyl)-N-hydroxybenzamide |
| 107 | 4-(((2S,6R)-4-(3-chlorobenzoyl)-2,6-dimethylpiperazin- |
| | 1-yl)methyl)-N-hydroxybenzamide |
| 108 | 4-(((2S,6R)-4-(2-chlorobenzoyl)-2,6-dimethylpiperazin- |
| | 1-yl)methyl)-N-hydroxybenzamide |
| 109 | 4-(((2S,6R)-4-((4-chlorophenyl)sulfonyl)-2,6- |
| | dimethylpiperazin-1-yl)methyl)-N-hydroxybenzamide |
| 110 | 4-(((2S,6R)-4-((2-chlorophenyl)sulfonyl)-2,6- |
| | dimethylpiperazin-1-yl)methyl)-N-hydroxybenzamide |
| 111 | 4-(((2S,6R)-2,6-dimethyl-4-picolinoylpiperazin-1- |
| | yl)methyl)-N-hydroxybenzamide |
| 112 | 4-(((2S,6R)-2,6-dimethyl-4-nicotinoylpiperazin-1- |
| | yl)methyl)-N-hydroxybenzamide |
| 113 | 4-(((2S,6R)-2,6-dimethyl-4-(pyridin-3-ylsulfonyl)piperazin- |
| | 1-yl)methyl)-N-hydroxybenzamide |
| 114 | 4-(((2S,6R)-2,6-dimethyl-4-(thiophene-2-carbonyl)piperazin- |
| | 1-yl)methyl)-N-hydroxybenzamide |
| 115 | 4-(((2S,6R)-4-(furan-2-carbonyl)-2,6-dimethylpiperazin- |
| | 1-yl)methyl)-N-hydroxybenzamide |
| 118 | 4-(((2S,6R)-4-(2-chlorobenzyl)-2,6-dimethylpiperazin- |
| | 1-yl)methyl)-N-hydroxybenzamide |
| 119 | 4-(((2R,6S)-4-(4-fluorophenyl)-2,6-dimethylpiperazin- |
| | 1-yl)methyl)-N-hydroxybenzamide |
| 120 | 4-(((3R,5S)-4-(2-chlorobenzoyl)-3,5-dimethylpiperazin- |
| | 1-yl)methyl)-N-hydroxybenzamide |
| 121 | 4-(((3R,5S)-4-(3-chlorobenzoyl)-3,5-dimethylpiperazin- |
| | 1-yl)methyl)-N-hydroxybenzamide |
| 122 | 4-(((3R,5S)-4-(4-chlorobenzoyl)-3,5-dimethylpiperazin- |
| | 1-yl)methyl)-N-hydroxybenzamide |
| 123 | 4-(((3R,5S)-3,5-dimethyl-4-picolinoylpiperazin- |
| | 1-yl)methyl)-N-hydroxybenzamide |
| 125 | 4-(((3R,5S)-4-(furan-2-carbonyl)-3,5-dimethylpiperazin- |
| | 1-yl)methyl)-N-hydroxybenzamide |
| 126 | 4-(((3R,5S)-3,5-dimethyl-4-(thiophene-2-carbonyl)piperazin- |
| | 1-yl)methyl)-N-hydroxybenzamide |
| 127 | 4-(((3R,5S)-4-(2-chlorobenzyl)-3,5-dimethylpiperazin- |
| | 1-yl)methyl)-N-hydroxybenzamide |
| 128 | 4-(((3R,5S)-4-(3-chlorobenzyl)-3,5-dimethylpiperazin- |
| | 1-yl)methyl)-N-hydroxybenzamide |
| 145 | 4-(((3R,5S)-4-(furan-2-ylmethyl)-3,5-dimethylpiperazin- |
| | 1-yl)methyl)-N-hydroxybenzamide |
| 146 | 4-(((3R,5S)-3,5-dimethyl-4-(2-phenylacetyl)piperazin- |
| | 1-yl)methyl)-N-hydroxybenzamide |
| 147 | 4-(((3R,5S)-4-ethyl-3,5-dimethylpiperazin-1-yl)methyl)- |
| | N-hydroxybenzamide |
| 148 | 4-(((3R,5S)-3,5-dimethyl-4-propylpiperazin-1-yl)methyl)- |
| | N-hydroxybenzamide |
| 149 | 4-(((3R,5S)-3,5-dimethyl-4-(2,2,2-trifluoroethyl)piperazin- |
| | 1-yl)methyl)-N-hydroxybenzamide |
| 154 | 6-((3R,5S)-4-benzyl-3,5-dimethylpiperazin-1-yl)- |
| | N-hydroxyhexanamide |
| 159 | (2S,6R)-4-(4-(hydroxycarbamoyl)benzyl)-2,6-dimethyl-N- |
| | phenylpiperazine-1-carboxamide |
| 160 | (2S,6R)-4-(4-(hydroxycarbamoyl)benzyl)-2,6-dimethyl-N-(o- |
| | tolyl)piperazine-1-carboxamide |
| 161 | (2S,6R)-N-benzyl-4-(4-(hydroxycarbamoyl)benzyl)-2,6- |
| | dimethylpiperazine-1-carboxamide |
| 162 | (3R,5S)-4-(4-(hydroxycarbamoyl)benzyl)-3,5-dimethyl-N- |
| | phenylpiperazine-1-carboxamide |
| 163 | (3R,5S)-4-(4-(hydroxycarbamoyl)benzyl)-N-(2-methoxyphenyl)- |
| | 3,5-dimethylpiperazine-1-carboxamide |
| 164 | (3R,5S)-4-(4-(hydroxycarbamoyl)benzyl)-N-(3-methoxyphenyl)- |
| | 3,5-dimethylpiperazine-1-carboxamide |
| 165 | (3R,5S)-4-(4-(hydroxycarbamoyl)benzyl)-N-(4-methoxyphenyl)- |
| | 3,5-dimethylpiperazine-1-carboxamide |
| 166 | (3R,5S)-4-(4-(hydroxycarbamoyl)benzyl)-3,5-dimethyl-N-(o- |
| | tolyl)piperazine-1-carboxamide |
| 167 | (3R,5S)-4-(4-(hydroxycarbamoyl)benzyl)-3,5-dimethyl-N-(m- |
| | tolyl)piperazine-1-carboxamide |
| 168 | (3R,5S)-N-benzyl-4-(4-(hydroxycarbamoyl)benzyl)-3,5- |
| | dimethylpiperazine-1-carboxamide |
| 171 | 4-(((3R,5S)-4-benzyl-3,5-dimethylpiperazin-1-yl)methyl)- |
| | 2-fluoro-N-hydroxybenzamide |
| 172 | (E)-3-(4-(((3R,5S)-4-benzyl-3,5-dimethylpiperazin-1- |
| | yl)methyl)phenyl)-N-hydroxyacrylamide |
| 173 | 2-(4-(((3R,5S)-4-benzyl-3,5-dimethylpiperazin-1- |
| | yl)methyl)phenyl)-N-hydroxyacetamide |
| 174 | 4-(((2S,6R)-4-benzyl-2,6-dimethylpiperazin-1- |
| | yl)methyl)-2-fluoro-N-hydroxybenzamide |
| 175 | (E)-3-(4-(((2S,6R)-4-benzyl-2,6-dimethylpiperazin-1-yl)methyl)phenyl)- |
| | N-hydroxyacrylamide |
| 176 | 2-(4-(((2S,6R)-4-benzyl-2,6-dimethylpiperazin-1-yl)methyl)phenyl)- |
| | N-hydroxyacetamide |
| 177 | phenyl |
| | (2S,6R)-4-(4-(hydroxycarbamoyl)benzyl)-2,6- |
| | dimethylpiperazine-1-carboxylate |
| 183 | 4-(((2S,6R)-2,6-dimethyl-4-phenethylpiperazin-1-yl)methyl)- |
| | N-hydroxybenzamide |
| 184 | 4-(((3R,5S)-3,5-dimethyl-4-phenethylpiperazin-1-yl)methyl)- |
| | N-hydroxybenzamide |
| 185 | (3R,5S)-4-(4-(hydroxycarbamoyl)benzyl)-3,5-dimethyl-N-(1-methyl- |
| | 1H-indol-5-yl)piperazine-1-carboxamide |
| 186 | (3R,5S)-N-(3-(1H-pyrrol-1-yl)phenyl)-4-(4-(hydroxycarbamoyl)benzyl)- |
| | 3,5-dimethylpiperazine-1-carboxamide |
| 187 | (3R,5S)-N-(2,3-dihydrobenzofuran-5-yl)-4-(4-(hydroxycarbamoyl)benzyl)- |
| | 3,5-dimethylpiperazine-1-carboxamide |
| 188 | 4-(((3R,5S)-3,5-dimethyl-4-(3-phenylpropanoyl)piperazin-1- |
| | yl)methyl)-N-hydroxybenzamide |
| 189 | 4-(((2S,6R)-2,6-dimethyl-4-(m-tolylcarbamothioyl)piperazin- |
| | 1-yl)methyl)-N-hydroxybenzamide |
| 193 | 4-(((2S,6R)-4-(2-fluoro-2-methylpropyl)-2,6-dimethylpiperazin- |
| | 1-yl)methyl)-N-hydroxybenzamide |
| 194 | 6-(((3R,5S)-4-benzyl-3,5-dimethylpiperazin-1-yl)methyl)- |
| | N-hydroxynicotinamide |
| 195 | 6-(((2S,6R)-4-benzyl-2,6-dimethylpiperazin-1-yl)methyl)- |
| | N-hydroxynicotinamide |
| 196 | (3R,5S)-4-(4-(hydroxycarbamoyl)benzyl)-N,3,5-trimethyl- |
| | N-phenylpiperazine-1-carboxamide |
| 197 | N-hydroxy-4-(((2S,6R)-4-(indoline-1-carbonyl)-2,6- |
| | dimethylpiperazin-1-yl)methyl)benzamide |
| 198 | (3R,5S)-N-butyl-4-(4-(hydroxycarbamoyl)benzyl)-3,5- |
| | dimethyl-N-phenylpiperazine-1-carboxamide |
| 204 | 4-(((3R,5S)-3,5-dimethyl-4-(4,4,4-trifluorobutyl)piperazin- |
| | 1-yl)methyl)-N-hydroxybenzamide |
| 211 | 4-(((3R,5S)-4-(2-fluorobenzyl)-3,5-dimethylpiperazin- |
| | 1-yl)methyl)-N-hydroxybenzamide |
| 212 | 4-(((3R,5S)-4-(2,5-difluorobenzyl)-3,5-dimethylpiperazin- |
| | 1-yl)methyl)-N-hydroxybenzamide |
| 213 | 4-(((3R,5S)-3,5-dimethyl-4-(2,4,5-trifluorobenzyl) |
| | piperazin-1-yl)methyl)-N-hydroxybenzamide |
| 214 | 4-(((3R,5S)-4-(3,5-bis(trifluoromethyl)benzyl)-3,5- |
| | dimethylpiperazin-1-yl)methyl)-N-hydroxybenzamide |
| 215 | 4-(((2S,6R)-2,6-dimethyl-4-(2,4,5-trifluorobenzyl) |
| | piperazin-1-yl)methyl)-N-hydroxybenzamide |
| 218 | 4-(2-((3R,5S)-4-benzyl-3,5-dimethylpiperazin-1-yl) |
| | ethyl)-N-hydroxybenzamide |
| 219 | 4-(1-((3R,5S)-4-benzyl-3,5-dimethylpiperazin-1-yl) |
| | ethyl)-N-hydroxybenzamide |
| 220 | 4-(((2S,6R)-2,6-dimethyl-4-(1-phenylethyl)piperazin- |
| | 1-yl)methyl)-N-hydroxybenzamide |
| 222 | 4-(((3R,5S)-4-(2-(3-fluorophenyl)acetyl)-3,5- |
| | dimethylpiperazin-1-yl)methyl)-N-hydroxybenzamide |
| 223 | 4-(((3R,5S)-3,5-dimethyl-4-(2-(3-(trifluoromethyl) |
| | phenyl)acetyl)piperazin-1-yl)methyl)-N- |
| | hydroxybenzamide |
| 224 | 4-(((3R,5S)-4-(2-(3-chlorophenyl)acetyl)-3,5- |
| | dimethylpiperazin-1-yl)methyl)-N-hydroxybenzamide |
| 225 | 4-(((3R,5S)-4-(3-fluorobenzyl)-3,5-dimethylpiperazin- |
| | 1-yl)methyl)-N-hydroxybenzamide |
| 230 | (3R,5S)-4-(4-(hydroxycarbamoyl)benzyl)-N-(3- |
| | methoxybenzyl)-3,5-dimethylpiperazine-1-carboxamide |
| | (Trifluoroacetic acid salt) |
| 231 | (3R,5S)-N-(3-fluorobenzyl)-4-(4-(hydroxycarbamoyl) |
| | benzyl)-3,5-dimethylpiperazine-1-carboxamide |
| 232 | (2S,6R)-N-(3-fluorobenzyl)-4-(4-(hydroxycarbamoyl) |
| | benzyl)-2,6-dimethylpiperazine-1-carboxamide |
| 233 | 4-(((2S,6R)-4-(2-(3-chlorophenyl)acetyl)-2,6- |
| | dimethylpiperazin-1-yl)methyl)-N-hydroxybenzamide |
| 234 | 4-(((3R,5S)-4-(2-([1,1′-biphenyl]-3-yl)acetyl)- |
| | 3,5-dimethylpiperazin-1-yl)methyl)-N-hydroxybenzamide |
| 242 | 4-(((3R,5S)-4-(3-(furan-2-yl)benzoyl)-3,5-dimethyl |
| | piperazin-1-yl)methyl)-N-hydroxybenzamide |
| 243 | 4-(((3R,5S)-4-(3-(furan-3-yl)benzoyl)-3,5-dimethyl |
| | piperazin-1-yl)methyl)-N-hydroxybenzamide |
| 244 | 4-(((3R,5S)-4-(3-(3,6-dihydro-2H-pyran-4-yl)benzoyl)- |
| | 3,5-dimethylpiperazin-1-yl)methyl)-N-hydroxybenzamide |
| 245 | 4-(((3R,5S)-3,5-dimethyl-4-(3-(pyridin-4- |
| | yl)benzoyl)piperazin-1-yl)methyl)-N- |
| | hydroxybenzamide (Trifluoroacetic acid salt) |
| 246 | 4-(((3R,5S)-3,5-dimethyl-4-(3-(pyridin-3- |
| | yl)benzoyl)piperazin-1-yl)methyl)-N-hydroxybenzamide |
| 247 | 4-(((3R,5S)-4-(4′,4′-dimethyl-2′,3′,4′,5′- |
| | tetrahydro-[1,1′-biphenyl]-3-carbonyl)-3,5-dimethylpiperazin- |
| | 1-yl)methyl)-N-hydroxybenzamide |
| 248 | 4-(((3R,5R)-4-benzyl-3,5-dimethylpiperazin-1- |
| | yl)methyl)-N-hydroxybenzamide |
| 249 | 4-(((3R,5R)-4-(furan-2-carbonyl)-3,5-dimethylpiperazin-1- |
| | yl)methyl)-N-hydroxybenzamide |
| 250 | 4-(((2S,6R)-4-(2-([1,1′-biphenyl]-3-yl)acetyl)- |
| | 2,6-dimethylpiperazin-1-yl)methyl)-N-hydroxybenzamide |
| | (Trifluoroacetic acid salt) |
| 251 | 4-(((2S,6R)-2,6-dimethyl-4-(2-(3-(pyridin-4- |
| | yl)phenyl)acetyl)piperazin-1-yl)methyl)-N-hydroxybenzamide |
| | (Trifluoroacetic acid salt) |
| 252 | 4-(((2S,6R)-4-(2-(3-(3,6-dihydro-2H-pyran-4- |
| | yl)phenyl)acetyl)-2,6-dimethylpiperazin-1-yl)methyl)- |
| | N-hydroxybenzamide |
| 253 | 4-(((2S,6R)-4-(2-(4′,4′-dimethyl-2′,3′,4′,5′- |
| | tetrahydro-[1,1′-biphenyl]-3-yl)acetyl)-2,6- |
| | dimethylpiperazin-1-yl)methyl)-N-hydroxybenzamide |
| | (Trifluoroacetic acid salt) |
| 255 | 4-(((3R,5S)-4-(3-(1H-pyrrol-1-yl)benzyl)-3,5- |
| | dimethylpiperazin-1-yl)methyl)-N-hydroxybenzamide |
| 256 | 4-(((2S,6R)-4-(3-(1H-pyrrol-1-yl)benzyl)-2,6- |
| | dimethylpiperazin-1-yl)methyl)-N-hydroxybenzamide |
| 257 | 4-(((2S,6R)-4-(3-(furan-2-yl)benzoyl)-2,6-dimethyl |
| | piperazin-1-yl)methyl)-N-hydroxybenzamide |
| 258 | 4-(((2S,6R)-4-(3-(furan-3-yl)benzoyl)-2,6-dimethyl |
| | piperazin-1-yl)methyl)-N-hydroxybenzamide |
| 259 | 4-(((2S,6R)-4-(3-(3,6-dihydro-2H-pyran-4- |
| | yl)benzoyl)-2,6-dimethylpiperazin-1-yl)methyl)- |
| | N-hydroxybenzamide |
| 260 | 4-(((2S,6R)-2,6-dimethyl-4-(3-(pyridin-4- |
| | yl)benzoyl)piperazin-1-yl)methyl)- |
| | N-hydroxybenzamide |
| 261 | 4-(((2S,6R)-2,6-dimethyl-4-(3-(pyridin-3- |
| | yl)benzoyl)piperazin-1-yl)methyl)- |
| | N-hydroxybenzamide |
| 262 | 4-(((2S,6R)-4-(3-(2-fluoropyridin-4-yl)benzoyl)- |
| | 2,6-dimethylpiperazin-1-yl)methyl)- |
| | N-hydroxybenzamide |
| 263 | 4-(((2S,6R)-4-(4′,4′-dimethyl-2′,3′,4′,5′- |
| | tetrahydro-[1,1′-biphenyl]-3-carbonyl)-2,6- |
| | dimethylpiperazin-1-yl)methyl)-N-hydroxybenzamide |
| 265 | 4-(((3R,5S)-3,5-dimethyl-4-(naphthalen-2-ylmethyl) |
| | piperazin-1-yl)methyl)-N-hydroxybenzamide |
| 266 | 4-(((3R,5S)-3,5-dimethyl-4-(pyridin-4-ylmethyl)piperazin- |
| | 1-yl)methyl)-N-hydroxybenzamide |
| | (Trifluoroacetic acid salt) |
| 267 | 4-(((3R,5S)-4-(3-chloro-2-(trifluoromethyl)benzyl)-3,5- |
| | dimethylpiperazin-1-yl)methyl)-N-hydroxybenzamide |
| 268 | 4-(((2S,6R)-2,6-dimethyl-4-(naphthalen-2-ylmethyl) |
| | piperazin-1-yl)methyl)-N-hydroxybenzamide |
| 270 | N-hydroxy-4-(((3R,5S)-4-isopentyl-3,5-dimethylpiperazin- |
| | 1-yl)methyl)benzamide (Trifluoroacetic acid salt) |
| 271 | N-hydroxy-4-(((2S,6R)-4-isopentyl-2,6-dimethylpiperazin- |
| | 1-yl)methyl)benzamide (Trifluoroacetic acid salt) |
| 272 | 4-(((3R,5S)-3,5-dimethyl-4-(3-methylbut-2-en-1-yl) |
| | piperazin-1-yl)methyl)-N-hydroxybenzamide |
| 273 | N-hydroxy-4-(((2S,6R)-4-isopropyl-2,6-dimethylpiperazin- |
| | 1-yl)methyl)benzamide (Trifluoroacetic acid salt) |
| 274 | 4-(((2S,6R)-4-butyl-2,6-dimethylpiperazin-1- |
| | yl)methyl)-N-hydroxybenzamide (Trifluoroacetic acid salt) |
| 275 | 4-(((3R,5S)-4-butyl-3,5-dimethylpiperazin-1- |
| | yl)methyl)-N-hydroxybenzamide (Trifluoroacetic acid salt) |
| 276 | 4-(((2S,6R)-4-(2-(furan-2-yl)benzoyl)-2,6-dimethyl |
| | piperazin-1-yl)methyl)-N-hydroxybenzamide |
| 277 | 4-(((2S,6R)-4-(2-(furan-3-yl)benzoyl)-2,6-dimethyl |
| | piperazin-1-yl)methyl)-N-hydroxybenzamide |
| 278 | 4-(((2S,6R)-4-(2-(3,6-dihydro-2H-pyran-4-yl)benzoyl)- |
| | 2,6-dimethylpiperazin-1-yl)methyl)-N-hydroxybenzamide |
| 279 | 4-(((2S,6R)-2,6-dimethyl-4-(2-(pyridin-3- |
| | yl)benzoyl)piperazin-1-yl)methyl)-N-hydroxybenzamide |
| 280 | 4-(((2S,6R)-4-(4′,4′-dimethyl-2′,3′,4′,5′- |
| | tetrahydro-[1,1′-biphenyl]-2-carbonyl)-2,6- |
| | dimethylpiperazin-1-yl)methyl)-N-hydroxybenzamide |
| 283 | 4-(((3R,5S)-4-(2-(furan-2-yl)benzoyl)-3,5-dimethyl |
| | piperazin-1-yl)methyl)-N-hydroxybenzamide |
| 284 | 4-(((3R,5S)-4-(2-(furan-3-yl)benzoyl)-3,5-dimethyl |
| | piperazin-1-yl)methyl)-N-hydroxybenzamide |
| 285 | 4-(((3R,5S)-3,5-dimethyl-4-(2-(pyridin-4- |
| | yl)benzoyl)piperazin-1-yl)methyl)-N-hydroxybenzamide |
| 286 | 4-(((3R,5S)-3,5-dimethyl-4-(2-(pyridin-3-yl)benzoyl)piperazin- |
| | 1-yl)methyl)-N-hydroxybenzamide |
| 288 | 4-(((3R,5S)-4-(4′,4′-dimethyl-2′,3′,4′,5′- |
| | tetrahydro-[1,1′-biphenyl]-2-carbonyl)-3,5- |
| | dimethylpiperazin-1-yl)methyl)-N-hydroxybenzamide |
| 290 | 4-(((3R,5S)-3,5-dimethyl-4-(4-methylbenzoyl)piperazin- |
| | 1-yl)methyl)-N-hydroxybenzamide (Trifluoroacetic |
| | acid salt) |
| 291 | N-hydroxy-4-(((3R,5S)-4-(4-methoxybenzoyl)-3,5-dimethylpiperazin- |
| | 1-yl)methyl)benzamide (Trifluoroacetic acid salt) |
| 292 | 4-(((3R,5S)-3,5-dimethyl-4-pivaloylpiperazin-1-yl)methyl)- |
| | N-hydroxybenzamide (Trifluoroacetic acid salt) |
| 293 | 4-(((3R,5S)-3,5-dimethyl-4-propionylpiperazin-1-yl)methyl)- |
| | N-hydroxybenzamide (Trifluoroacetic acid salt) |
| 294 | 4-(((3R,5S)-4-butyryl-3,5-dimethylpiperazin-1-yl)methyl)- |
| | N-hydroxybenzamide (Trifluoroacetic acid salt) |
| 295 | 4-(((3R,5S)-4-(cyclopropanecarbonyl)-3,5-dimethylpiperazin- |
| | 1-yl)methyl)-N-hydroxybenzamide (Trifluoroacetic acid salt) |
| 296 | N-hydroxy-4-(((3R,5S)-4-isobutyryl-3,5-dimethylpiperazin- |
| | 1-yl)methyl)benzamide (Trifluoroacetic acid salt) |
| 297 | 4-(((3R,5S)-3,5-dimethyl-4-(3-methylbutanoyl)piperazin- |
| | 1-yl)methyl)-N-hydroxybenzamide (Trifluoroacetic acid salt) |
| 298 | 4-(((2S,6R)-2,6-dimethyl-4-(3-(trifluoromethyl)benzyl)piperazin- |
| | 1-yl)methyl)-N-hydroxybenzamide |
| 299 | 4-(((2S,6R)-4-(4-(dimethylamino)benzyl)-2,6- |
| | dimethylpiperazin-1-yl)methyl)-N-hydroxybenzamide |
| 300 | 4-(((2S,6R)-2,6-dimethyl-4-(4-(methylsulfonyl)benzyl)piperazin- |
| | 1-yl)methyl)-N-hydroxybenzamide |
| 301 | 4-(((2S,6R)-4-(4-(1H-imidazol-1-yl)benzyl)-2,6- |
| | dimethylpiperazin-1-yl)methyl)-N-hydroxybenzamide |
| 302 | 4-(((2S,6R)-2,6-dimethyl-4-(4-(thiophen-2- |
| | yl)benzyl)piperazin-1-yl)methyl)-N-hydroxybenzamide |
| 303 | 4-(((2S,6R)-4-(4-(furan-2-yl)benzyl)-2,6- |
| | dimethylpiperazin-1-yl)methyl)-N-hydroxybenzamide |
| 304 | 4-(((2S,6R)-4-(4-(4H-1,2,4-triazol-4-yl)benzyl)-2,6- |
| | dimethylpiperazin-1-yl)methyl)-N-hydroxybenzamide |
| 305 | 4-(((2S,6R)-2,6-dimethyl-4-(2-(2-methyl-1H-imidazol- |
| | 1-yl)benzyl)piperazin-1-yl)methyl)-N-hydroxybenzamide |
| 309 | 4-(((3R,5S)-4-(4-(furan-3-yl)benzyl)-3,5- |
| | dimethylpiperazin-1-yl)methyl)-N-hydroxybenzamide |
| 321 | (2S,6R)-N-(3-(1H-pyrrol-1-yl)phenyl)-4-(4-((E)-3- |
| | (hydroxyamino)-3-oxoprop-1-en-1-yl)benzyl)-2,6- |
| | dimethylpiperazine-1-carboxamide |
| 322 | (E)-3-(4-(((3R,5S)-4-(furan-2-carbonyl)-3,5- |
| | dimethylpiperazin-1-yl)methyl)phenyl)-N-hydroxyacrylamide |
| 323 | (E)-3-(4-(((3R,5S)-3,5-dimethyl-4-(2-phenylacetyl) |
| | piperazin-1-yl)methyl)phenyl)-N-hydroxyacrylamide |
| 326 | 4-(((3R,5S)-4-(2-(3,6-dihydro-2H-pyran-4-yl)benzoyl)- |
| | 3,5-dimethylpiperazin-1-yl)methyl)-N-hydroxybenzamide |
| 327 | 4-(((3R,5S)-4-(2-(furan-2-yl)benzyl)-3,5-dimethylpiperazin- |
| | 1-yl)methyl)-N-hydroxybenzamide |
| 328 | 4-(((3R,5S)-4-(2-(furan-3-yl)benzyl)-3,5-dimethylpiperazin- |
| | 1-yl)methyl)-N-hydroxybenzamide |
| 329 | 4-(((3R,5S)-3,5-dimethyl-4-(2-(pyridin-4-yl)benzyl)piperazin- |
| | 1-yl)methyl)-N-hydroxybenzamide |
| 330 | 4-(((3R,5S)-3,5-dimethyl-4-(2-(pyridin-3-yl)benzyl)piperazin- |
| | 1-yl)methyl)-N-hydroxybenzamide |
| 331 | 4-(((3R,5S)-4-((4′,4′-dimethyl-2′,3′,4′,5′- |
| | tetrahydro-[1,1′-biphenyl]-2-yl)methyl)-3,5- |
| | dimethylpiperazin-1-yl)methyl)-N-hydroxybenzamide |
| 332 | 4-(((3R,5S)-4-(2-(3,6-dihydro-2H-pyran-4-yl)benzyl)-3,5- |
| | dimethylpiperazin-1-yl)methyl)-N-hydroxybenzamide |
| 340 | 4-(((3R,5S)-4-(4-(furan-2-yl)benzoyl)-3,5- |
| | dimethylpiperazin-1-yl)methyl)-N-hydroxybenzamide |
| 342 | 4-(((3R,5S)-4-(3-(furan-2-yl)benzyl)-3,5- |
| | dimethylpiperazin-1-yl)methyl)-N-hydroxybenzamide |
| 343 | 4-(((3R,5S)-4-(3-(furan-3-yl)benzyl)-3,5- |
| | dimethylpiperazin-1-yl)methyl)-N-hydroxybenzamide |
| 344 | 4-(((3R,5S)-3,5-dimethyl-4-(3-(pyridin-3-yl)benzyl)piperazin-1- |
| | yl)methyl)-N-hydroxybenzamide |
| 345 | 4-(((3R,5S)-3,5-dimethyl-4-(3-(pyridin-4-yl)benzyl)piperazin-1- |
| | yl)methyl)-N-hydroxybenzamide |
| 346 | 4-(((3R,5S)-4-(3-(3,6-dihydro-2H-pyran-4-yl)benzyl)-3,5- |
| | dimethylpiperazin-1-yl)methyl)-N-hydroxybenzamide |
| 347 | 4-(((3R,5S)-4-((4′,4′-dimethyl-2′,3′,4′,5′- |
| | tetrahydro-[1,1′-biphenyl]-3-yl)methyl)-3,5- |
| | dimethylpiperazin-1-yl)methyl)-N-hydroxybenzamide |
| 348 | 4-(((2S,6R)-4-(2-(furan-3-yl)benzyl)-2,6- |
| | dimethylpiperazin-1-yl)methyl)-N-hydroxybenzamide |
| 349 | 4-(((2S,6R)-2,6-dimethyl-4-(2-(pyridin-3-yl)benzyl)piperazin- |
| | 1-yl)methyl)-N-hydroxybenzamide |
| 350 | 4-(((2S,6R)-2,6-dimethyl-4-(2-(pyridin-4-yl)benzyl)piperazin- |
| | 1-yl)methyl)-N-hydroxybenzamide |
| 351 | 4-(((2S,6R)-4-(2-(3,6-dihydro-2H-pyran-4-yl)benzyl)-2,6- |
| | dimethylpiperazin-1-yl)methyl)-N-hydroxybenzamide |
| 352 | 4-(((2S,6R)-4-((4′,4′-dimethyl-2′,3′,4′,5′- |
| | tetrahydro-[1,1′-biphenyl]-2-yl)methyl)-2,6- |
| | dimethylpiperazin-1-yl)methyl)-N-hydroxybenzamide |
| 354 | 4-(((3R,5S)-3,5-dimethyl-4-(oxazol-4-ylmethyl)piperazin- |
| | 1-yl)methyl)-N-hydroxybenzamide |
| 355 | 4-(((3R,5S)-3,5-dimethyl-4-(pyrimidin-5-ylmethyl)piperazin- |
| | 1-yl)methyl)-N-hydroxybenzamide |
| 356 | 4-(((3R,5S)-3,5-dimethyl-4-(3-(morpholinomethyl)benzyl)piperazin- |
| | 1-yl)methyl)-N-hydroxybenzamide |
| 376 | 4-(((3R,5S)-3,5-dimethyl-4-(4-(morpholinomethyl)benzyl)piperazin- |
| | 1-yl)methyl)-N-hydroxybenzamide |
| 380 | 4-(((3R,5S)-4-(3-((1-(2-fluoro-2-methylpropyl)piperidin- |
| | 4-yl)methoxy)benzyl)-3,5-dimethylpiperazin-1-yl)methyl)- |
| | N-hydroxybenzamide |
| 382 | 4-(((3R,5S)-3,5-dimethyl-4-(3-(piperidin-1- |
| | ylmethyl)benzyl)piperazin-1-yl)methyl)- |
| | N-hydroxybenzamide |
| 383 | 4-(((3R,5S)-4-(3-(((R)-3-fluoropyrrolidin-1- |
| | yl)methyl)benzyl)-3,5-dimethylpiperazin-1-yl)methyl)- |
| | N-hydroxybenzamide |
| 384 | 4-(((3R,5S)-4-(3-((3,3-difluoroazetidin-1- |
| | yl)methyl)benzyl)-3,5-dimethylpiperazin-1-yl)methyl)- |
| | N-hydroxybenzamide |
| 385 | 4-(((3R,5S)-4-(3-((4-benzylpiperazin-1- |
| | yl)methyl)benzyl)-3,5-dimethylpiperazin-1-yl)methyl)- |
| | N-hydroxybenzamide |
| 386 | N-hydroxy-4-(((3R,5S)-4-(3-((4-(2-hydroxy-2-methyl |
| | propyl)piperazin-1-yl)methyl)benzyl)-3,5- |
| | dimethylpiperazin-1-yl)methyl)benzamide |
| 387 | 4-(((3R,5S)-4-(3-((4-(2-fluoro-2-methylpropyl)piperazin- |
| | 1-yl)methyl)benzyl)-3,5-dimethylpiperazin-1-yl)methyl)- |
| | N-hydroxybenzamide |
| 388 | 4-(((3R,5S)-4-(4-((1-(2-fluoro-2-methylpropyl)piperidin- |
| | 4-yl)methoxy)benzyl)-3,5-dimethylpiperazin-1-yl)methyl)- |
| | N-hydroxybenzamide |
| 396 | 4-(((2S,6R)-4-(3-(furan-3-yl)benzyl)-2,6-dimethylpiperazin- |
| | 1-yl)methyl)-N-hydroxybenzamide |
| 400 | 4-(((2S,6R)-4-((4′,4′-dimethyl-2′,3′,4′,5′- |
| | tetrahydro-[1,1′-biphenyl]-4-yl)methyl)-2,6- |
| | dimethylpiperazin-1-yl)methyl)-N-hydroxybenzamide |
| 401 | 4-(((2S,6R)-4-(4-(3,6-dihydro-2H-pyran-4-yl)benzyl)- |
| | 2,6-dimethylpiperazin-1-yl)methyl)-N-hydroxybenzamide |
| 402 | 4-(((3R,5S)-3,5-dimethyl-4-(tetrahydrofuran-2- |
| | carbonyl)piperazin-1-yl)methyl)-N-hydroxybenzamide |
| 403 | 4-(((3R,5S)-3,5-dimethyl-4-(1H-pyrazole-3-carbonyl)piperazin- |
| | 1-yl)methyl)-N-hydroxybenzamide |
| 404 | 4-(((3R,5S)-3,5-dimethyl-4-(pyrazine-2-carbonyl)piperazin- |
| | 1-yl)methyl)-N-hydroxybenzamide |
| 405 | N-hydroxy-4-(((3R,5S)-4-isonicotinoyl-3,5-dimethyl |
| | piperazin-1-yl)methyl)benzamide |
| 411 | 4-(((3R,5S)-4-(4-(((R)-3-fluoropyrrolidin-1-yl)methyl)benzyl)- |
| | 3,5-dimethylpiperazin-1-yl)methyl)-N-hydroxybenzamide |
| 412 | 4-(((3R,5S)-4-(4-((3,3-difluoroazetidin-1- |
| | yl)methyl)benzyl)-3,5-dimethylpiperazin-1-yl)methyl)- |
| | N-hydroxybenzamide |
| 413 | 4-(((3R,5S)-4-(4-((4-benzylpiperazin-1-yl)methyl)benzyl)- |
| | 3,5-dimethylpiperazin-1-yl)methyl)-N-hydroxybenzamide |
| 423 | 4-(((3R,5S)-3,5-dimethyl-4-(3-(pyrrolidin-1- |
| | ylmethyl)benzoyl)piperazin-1-yl)methyl)-N-hydroxybenzamide |
| 424 | 4-(((3R,5S)-3,5-dimethyl-4-(3-(piperidin-1- |
| | ylmethyl)benzoyl)piperazin-1-yl)methyl)-N-hydroxybenzamide |
| 425 | 4-(((3R,5S)-4-(3-((diethylamino)methyl)benzoyl)- |
| | 3,5-dimethylpiperazin-1-yl)methyl)-N-hydroxybenzamide |
| 426 | 4-(((3R,5S)-4-(3-((diethylamino)methyl)benzyl)- |
| | 3,5-dimethylpiperazin-1-yl)methyl)-N-hydroxybenzamide |
| 427 | 4-(((3R,5S)-3,5-dimethyl-4-(3-(pyrrolidin-1- |
| | ylmethyl)benzyl)piperazin-1-yl)methyl)-N-hydroxybenzamide |
| 428 | 4-(((3R,5S)-3,5-dimethyl-4-(3-((4-methylpiperazin-1- |
| | yl)methyl)benzyl)piperazin-1-yl)methyl)-N-hydroxybenzamide |
| 429 | 4-(((3R,5S)-4-(3-((4-ethylpiperazin-1-yl)methyl)benzyl)- |
| | 3,5-dimethylpiperazin-1-yl)methyl)-N-hydroxybenzamide |
| 430 | N-hydroxy-4-(((3R,5S)-4-(3-((4-isopropylpiperazin-1- |
| | yl)methyl)benzyl)-3,5-dimethylpiperazin-1-yl)methyl)benzamide |
| 431 | 4-(((3R,5S)-4-(3-((4-acetylpiperazin-1-yl)methyl)benzyl)- |
| | 3,5-dimethylpiperazin-1-yl)methyl)-N-hydroxybenzamide |
| 432 | 4-(((3R,5S)-3,5-dimethyl-4-(3-((4-methylpiperazin-1- |
| | yl)methyl)benzoyl)piperazin-1-yl)methyl)-N-hydroxybenzamide |
| 433 | 4-(((3R,5S)-4-(3-((4-ethylpiperazin-1-yl)methyl)benzoyl)- |
| | 3,5-dimethylpiperazin-1-yl)methyl)-N-hydroxybenzamide |
| 434 | N-hydroxy-4-(((3R,5S)-4-(3-((4-isopropylpiperazin-1- |
| | yl)methyl)benzoyl)-3,5-dimethylpiperazin-1- |
| | yl)methyl)benzamide |
| 435 | 4-(((3R,5S)-4-(3-((4-acetylpiperazin-1-yl)methyl)benzoyl)- |
| | 3,5-dimethylpiperazin-1-yl)methyl)-N-hydroxybenzamide |
| 439 | 4-(((3R,5S)-3,5-dimethyl-4-(3-(morpholinomethyl)benzoyl)piperazin- |
| | 1-yl)methyl)-N-hydroxybenzamide |
| 440 | 4-(((3R,5S)-4-(3-(((1-(diethylamino)propan-2-yl)amino)methyl)benzoyl)- |
| | 3,5-dimethylpiperazin-1-yl)methyl)-N-hydroxybenzamide |
| 441 | 4-(((3R,5S)-4-(3-(((1-(benzyl(methyl)amino)propan-2- |
| | yl)amino)methyl)benzoyl)-3,5-dimethylpiperazin-1-yl)methyl)- |
| | N-hydroxybenzamide |
| 442 | N-hydroxy-4-(((3R,5S)-4-(3-((4-isopentylpiperazin-1- |
| | yl)methyl)benzoyl)-3,5-dimethylpiperazin-1-yl)methyl)benzamide |
| 443 | 4-(((3R,5S)-3,5-dimethyl-4-(3-((4-(2,2,2-trifluoroethyl)piperazin- |
| | 1-yl)methyl)benzoyl)piperazin-1-yl)methyl)-N-hydroxybenzamide |
| 444 | 4-(((3R,5S)-3,5-dimethyl-4-(3-((4-(methylsulfonyl)piperazin- |
| | 1-yl)methyl)benzoyl)piperazin-1-yl)methyl)-N-hydroxybenzamide |
| 446 | 4-(((3R,5S)-3,5-dimethyl-4-(3-(morpholine-4- |
| | carbonyl)benzyl)piperazin-1-yl)methyl)-N-hydroxybenzamide |
| 452 | 4-(((3R,5S)-3,5-dimethyl-4-(3-((4-(methylsulfonyl)piperazin- |
| | 1-yl)methyl)benzyl)piperazin-1-yl)methyl)-N-hydroxybenzamide |
| 453 | 4-(((3R,5S)-3,5-dimethyl-4-(3-((4-(2,2,2-trifluoroethyl)piperazin- |
| | 1-yl)methyl)benzyl)piperazin-1-yl)methyl)-N-hydroxybenzamide |
| 454 | N-hydroxy-4-(((3R,5S)-4-(3-((4-isopentylpiperazin-1- |
| | yl)methyl)benzyl)-3,5-dimethylpiperazin-1-yl)methyl)benzamide |
| 455 | 4-(((3R,5S)-4-(3-(((1-(diethylamino)propan-2-yl)amino)methyl)benzyl)- |
| | 3,5-dimethylpiperazin-1-yl)methyl)-N-hydroxybenzamide |
| 456 | 4-(((3R,5S)-4-(3-(((1-(benzyl(methyl)amino)propan-2- |
| | yl)amino)methyl)benzyl)-3,5-dimethylpiperazin-1-yl)methyl)- |
| | N-hydroxybenzamide |
| 457 | 4-(((3R,5S)-3,5-dimethyl-4-(2-(morpholinomethyl)benzyl)piperazin- |
| | 1-yl)methyl)-N-hydroxybenzamide |
| 458 | 4-(((3R,5S)-3,5-dimethyl-4-(4-(morpholinomethyl)benzoyl)piperazin- |
| | 1-yl)methyl)-N-hydroxybenzamide |
| 459 | 4-(((3R,5S)-3,5-dimethyl-4-(4-(piperidin-1-ylmethyl)benzoyl)piperazin- |
| | 1-yl)methyl)-N-hydroxybenzamide |
| 460 | 4-(((3R,5S)-4-(4-(((R)-3-fluoropyrrolidin-1-yl)methyl)benzoyl)- |
| | 3,5-dimethylpiperazin-1-yl)methyl)-N-hydroxybenzamide |
| 461 | 4-(((3R,5S)-3,5-dimethyl-4-(4-(pyrrolidin-1- |
| | ylmethyl)benzoyl)piperazin-1-yl)methyl)-N-hydroxybenzamide |
| 462 | 4-(((3R,5S)-3,5-dimethyl-4-(4-((4-methylpiperazin-1- |
| | yl)methyl)benzoyl)piperazin-1-yl)methyl)-N- |
| | hydroxybenzamide |
| 463 | 4-(((3R,5S)-3,5-dimethyl-4-(4-((4-(methylsulfonyl)piperazin- |
| | 1-yl)methyl)benzoyl)piperazin-1-yl)methyl)-N-hydroxybenzamide |
| 466 | 4-(((3R,5S)-3,5-dimethyl-4-(4-(pyrrolidin-1- |
| | ylmethyl)benzyl)piperazin-1-yl)methyl)-N-hydroxybenzamide |
| 467 | 4-(((3R,5S)-3,5-dimethyl-4-(4-(piperidin-1-ylmethyl)benzyl)piperazin- |
| | 1-yl)methyl)-N-hydroxybenzamide |
| 468 | 4-(((3R,5S)-3,5-dimethyl-4-(4-((4-methylpiperazin-1- |
| | yl)methyl)benzyl)piperazin-1-yl)methyl)-N- |
| | hydroxybenzamide |
| 472 | (E)-3-(4-(((3R,5S)-3,5-dimethyl-4-(3-(morpholinomethyl)benzyl)piperazin- |
| | 1-yl)methyl)phenyl)-N-hydroxyacrylamide |
| 475 | (E)-3-(4-(((2S,6R)-2,6-dimethyl-4-(3-(pyrrolidin-1- |
| | ylmethyl)benzyl)piperazin-1-yl)methyl)phenyl)-N- |
| | hydroxyacrylamide |
| 476 | (E)-3-(4-(((2S,6R)-4-(3-((diethylamino)methyl)benzyl)- |
| | 2,6-dimethylpiperazin-1-yl)methyl)phenyl)-N- |
| | hydroxyacrylamide |
| 477 | (E)-3-(4-(((2S,6R)-4-(4-(furan-2-yl)benzyl)-2,6- |
| | dimethylpiperazin-1-yl)methyl)phenyl)-N- |
| | hydroxyacrylamide |
| 478 | 4-(((2S,6R)-4-(3-((diethylamino)methyl)benzyl)- |
| | 2,6-dimethylpiperazin-1-yl)methyl)-N-hydroxybenzamide |
| | (hydrochloride salt) |
| 479 | 4-(((2S,6R)-2,6-dimethyl-4-(3-(pyrrolidin-1- |
| | ylmethyl)benzyl)piperazin-1-yl)methyl)-N-hydroxybenzamide |
| 480 | (E)-3-(4-(((2S,6R)-2,6-dimethyl-4-(3-(pyrrolidin-1- |
| | ylmethyl)benzoyl)piperazin-1-yl)methyl)phenyl)- |
| | N-hydroxyacrylamide |
| 481 | 4-(((3R,5S)-3,5-dimethyl-4-(2-(1-(methylsulfonyl)-1,2,3,6- |
| | tetrahydropyridin-4-yl)benzyl)piperazin-1-yl)methyl)- |
| | N-hydroxybenzamide |
| 482 | 4-(((3R,5S)-4-(2-(1-acetyl-1,2,3,6-tetrahydropyridin- |
| | 4-yl)benzyl)-3,5-dimethylpiperazin-1-yl)methyl)-N-hydroxybenzamide |
| 483 | 4-(((3R,5S)-3,5-dimethyl-4-(2-(1-(2,2,2-trifluoroethyl)- |
| | 1,2,3,6-tetrahydropyridin-4-yl)benzyl)piperazin-1-yl)methyl)- |
| | N-hydroxybenzamide |
| 484 | N-hydroxy-4-(((3R,5S)-4-(2-(1-isopropyl-1,2,3,6-tetrahydropyridin- |
| | 4-yl)benzyl)-3,5-dimethylpiperazin-1-yl)methyl)benzamide |
| 485 | 4-(((3R,5S)-4-(2-(1-ethyl-1,2,3,6-tetrahydropyridin- |
| | 4-yl)benzyl)-3,5-dimethylpiperazin-1-yl)methyl)-N- |
| | hydroxybenzamide |
| 486 | 4-(((3R,5S)-4-(4-((4-acetylpiperazin-1-yl)methyl)benzyl)- |
| | 3,5-dimethylpiperazin-1-yl)methyl)-N- |
| | hydroxybenzamide (hydrochloride salt) |
| 487 | (E)-3-(4-(((2S,6R)-4-(3-((diethylamino)methyl)benzoyl)- |
| | 2,6-dimethylpiperazin-1-yl)methyl)phenyl)- |
| | N-hydroxyacrylamide |
| 520 | 3-(((3R,5S)-4-benzyl-3,5-dimethylpiperazin-1- |
| | yl)methyl)-N-hydroxy-1H-indole-6-carboxamide |
| 569 | 5-(((3R,5S)-4-benzyl-3,5-dimethylpiperazin-1- |
| | yl)methyl)-N-hydroxy-1H-indole-2-carboxamide |
| 571 | 5-(((3R,5S)-4-benzyl-3,5-dimethylpiperazin-1- |
| | yl)methyl)-N-hydroxybenzofuran-2-carboxamide |
| 573 | 6-(((3R,5S)-4-benzyl-3,5-dimethylpiperazin-1- |
| | yl)methyl)-N-hydroxy-1H-indole-2-carboxamide |
| 574 | 2-(((3R,5S)-4-benzyl-3,5-dimethylpiperazin-1- |
| | yl)methyl)-N-hydroxybenzofuran-5-carboxamide |
| 609 | 5-(((3R,5S)-3,5-dimethyl-4-(3- |
| | (morpholinomethyl)benzyl)piperazin-1- |
| | yl)methyl)-N-hydroxypicolinamide |
| 652 | 4-(((3R,5S)-4-benzyl-3,5-dimethylpiperazin-1- |
| | yl)methyl)-N,2-dihydroxybenzamide |
| 653 | 4-(((3R,5S)-3,5-dimethyl-4-(3- |
| | (morpholinomethyl)benzyl)piperazin-1- |
| | yl)methyl)-N,2-dihydroxybenzamide |
| 696 | 4-(((3R,5S)-3,5-dimethyl-4-(3- |
| | (morpholinomethyl)benzyl)piperazin-1- |
| | yl)methyl)-3-fluoro-N-hydroxybenzamide |
| 812 | 5-(((3R,5S)-4-benzyl-3,5-dimethylpiperazin-1- |
| | yl)methyl)-N-hydroxypicolinamide |
| 813 | 4-(((3R,5S)-4-benzyl-3,5-dimethylpiperazin-1- |
| | yl)methyl)-3-fluoro-N-hydroxybenzamide |
| 814 | 4-((3R,5S)-4-benzyl-3,5-dimethylpiperazine-1- |
| | carbonyl)-N-hydroxybenzamide |
| 818 | 4-(((3R,5S)-4-benzyl-3,5-dimethylpiperazin-1- |
| | yl)sulfonyl)-N-hydroxybenzamide |
| 820 | 2-(4-(((3R,5S)-4-benzyl-3,5-dimethylpiperazin- |
| | 1-yl)sulfonyl)phenyl)-N-hydroxyacetamide |
| 822 | 4-(((3R,5S)-4-benzyl-3,5-dimethylpiperazin-1- |
| | yl)methyl)-N-hydroxycyclohexane-1-carboxamide |
| 823 | 2-(4-((3R,5S)-4-benzyl-3,5-dimethylpiperazine-1- |
| | carbonyl)phenyl)-N-hydroxyacetamide |
| 824 | 3-(4-((3R,5S)-4-benzyl-3,5-dimethylpiperazine-1- |
| | carbonyl)phenyl)-N-hydroxypropanamide |

Preferably, the compounds represented by formula I or pharmaceutically acceptable salts thereof according to the present invention may be selected from the group consisting of compounds 080, 081, 082, 083, 084, 098, 099, 100, 106, 107, 108, 109, 110, 112, 120, 121, 122, 123, 125, 126, 127, 128, 145, 146, 147, 148, 149, 159, 160, 161, 171, 173, 174, 175, 177, 186, 188, 193, 194, 195, 196, 198, 211, 214, 219, 248, 249, 250, 251, 252, 255, 265, 266, 267, 272, 283, 284, 285, 286, 292, 295, 297, 305, 326, 328, 329, 330, 332, 342, 343, 344, 345, 346, 349, 354, 356, 376, 380, 382, 383, 384, 385, 386, 387, 388, 403, 411, 413, 430, 431, 432, 433, 434, 435, 439, 440, 441, 442, 443, 444, 452, 453, 454, 455, 456, 467, 468, 481, 482, 483, 484, 485, 486, 569, 696, 813 and 823. More preferably, the compounds represented by formula I or pharmaceutically acceptable salts thereof according to the present invention may be selected from the group consisting of compounds 082, 083, 084, 098, 100, 120, 121, 122, 123, 125, 126, 127, 128, 145, 146, 148, 149, 159, 160, 161, 171, 174, 177, 194, 211, 249, 255, 283, 305, 326, 328, 329, 330, 332, 342, 343, 344, 345, 346, 349, 354, 356, 376, 382, 383, 387, 388, 411, 413, 431, 439, 441, 444, 452, 453, 454, 467, 468, 481, 482, 483, 484, 485, 696 and 813.

As used herein, the term “pharmaceutically acceptable salt” means any salt that is generally used in the pharmaceutical field. Examples of the pharmaceutically acceptable salt include, but are not limited to, salts with inorganic ions such as calcium, potassium, sodium or magnesium ions, salts with inorganic acids such as hydrochloric acid, nitric acid, phosphoric acid, bromic acid, iodic acid, perchloric acid, tartaric acid or sulfuric acid, salts with organic acids such as acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid or the like, salts with sulfonic acids such as methanesulfonic acid, ethanesulfonic acid, benzenesulfonic acid, p-toluenesulfonic acid or naphthalenesulfonic acid, salts with amino acids such as glycine, arginine or lysine, and salts with amines such as trimethylamine, tri-ethylamine, ammonia, pyridine or picoline.

In the present invention, preferred salts include salts with hydrochloric acid, trifluoroacetic acid, citric acid, bromic acid, maleic acid, phosphoric acid, sulfuric acid, tartaric acid or the like, and preferred examples of such compounds include compounds 230, 245, 250, 251, 253, 266, 270, 271, 273, 274, 275, 290, 291, 292, 293, 294, 295, 296, 297, 478 and 486 as disclosed herein.

The compounds represented by formula I may contain one or more asymmetrical carbon atoms, and thus may exist in the form of racemates, racemic mixtures, single enantiomers (optical isomer), diastereomeric mixtures, and individual diastereomers. The compounds of formula I can be separated into such isomers by methods known in the art, for example, column chromatography or HPLC. Alternatively, stereoisomers of the compounds of formula I may be synthesized by stereospecific synthesis using optically pure starting materials and/or reagents of known configuration. Particularly, the isomers may be optical isomers.

In the present invention, the compound represented by Formula I may be prepared by means of a method disclosed in Korean Patent Registration No. 1697518, but is not limited thereto.

A pharmaceutical composition comprising histone deacetylase 6 inhibitor of the present invention is used in preventing or treating CMT disease.

Charcot-Marie-Tooth (CMT) disease (hereditary motor and sensory neuropathy: HMSN) is the most common type of hereditary peripheral nerve disorder, which is known to be mainly caused by a defect in axonal transport.

In the present invention, CMT disease may be at least one selected from the group consisting of CMT type 1, CMT type 2, CMT type 4, CMTX, DSN, CH (congenital hypomyelination), HNPP (hereditary neuropathy with liability to pressure palsy) and GAN (giant axonal neuropathy), but is not limited thereto.

In the present invention, CMT disease may be prevented or treated by means of administration of the pharmaceutical composition according to the present invention. For example, the pharmaceutical composition according to the present invention may prevent or treat CMT disease associated with histone deacetylase 6 activity by containing a novel dimethylpiperazine derivative compound having histone deacetylase 6 (HDAC6) inhibitory activity, and thus by improving axonal transport.

In one embodiment of the present invention, the compound represented by Formula I selectively has an inhibitory activity to the histone deacetylase 6 (HDAC6), and thus may alleviate a decline in motor ability and improve an electrophysiological index, may treat or alleviate symptoms caused by CMT disease, such as axonal atrophy and loss of myelinated nerves, and may inhibit or delay an expression of such symptoms. Particularly, the compound represented by Formula I according to the present invention may effectively treat or alleviate symptoms shown in CMT disease, such as a decrease in motor nerve conduction velocity, a decrease in compound muscle action potential, ongoing neuronal degeneration, muscle weakness, abnormal sense, schwann cell hyperplasia, loss of myelinated nerves and axonal atrophy, and may inhibit or delay an expression of such symptoms.

For administration, the pharmaceutical composition according to the present invention may further contain at least one pharmaceutically acceptable carrier in addition to the compound of formula I, an isomer thereof or a pharmaceutically acceptable salt thereof. The pharmaceutically acceptable carrier that is used in the present invention may be at least one of physiological saline, sterile water, Ringer solution, buffered saline, dextrose solution, maltodextrin solution, glycerol, ethanol, and a mixture of one or more thereof. If necessary, the composition may contain other conventional additives such as an antioxidant, a buffer or a bacteriostatic agent. In addition, the composition can be formulated into injectable formulations such as solutions, suspensions, turbid fluid, etc, pills, capsules, granules and tablets using a diluent, a dispersing agent, a surfactant, a binder and a lubricant. Thus, the composition of the present invention may be in the form of patches, liquids, pills, capsules, granules, tablets, suppositories, etc. These formulations can be prepared either by conventional methods that are used for formulation in the art or by the method disclosed in Remington's Pharmaceutical Science (the latest edition), Mack Publishing Company, Easton Pa. In addition, the composition of the present invention can be prepared as various formulations depending on diseases or components.

As a non-limiting example of preparations for oral administration using the pharmaceutical composition of the present invention, there may be tablets, troches, lozenges, water-soluble suspensions, oil suspensions, prepared powders, granules, emulsions, hard capsules, soft capsules, syrups, elixirs or the like. To formulate the pharmaceutical composition of the present invention into preparations for oral administration, the followings may be used: binders such as lactose, saccharose, sorbitol, mannitol, starch, amylopectin, cellulose, gelatin or the like; excipients such as dicalcium phosphate, etc.; disintegrants such as maize starch, sweet potato starch or the like; lu-bricants such as magnesium stearate, calcium stearate, sodium stearyl fumarate, polyethylene glycol wax or the like; etc., wherein sweetening agents, flavoring agents, syrups, etc. may also be used. Furthermore, in case of the capsules, liquid carriers such as fatty oil, etc. may be further used in addition to the above-mentioned materials.

As a non-limiting example of parenteral preparations using the pharmaceutical composition of the present invention, there may be injectable solutions, suppositories, powders for respiratory inhalation, aerosols for spray, ointments, powders for ap-plication, oils, creams, etc. To formulate the pharmaceutical composition of the present invention into preparations for parenteral administration, the followings may be used: sterilized aqueous solutions, nonaqueous solvents, suspensions, emulsions, freeze-dried preparations, external preparations, etc. As the nonaqueous solvents and suspensions, the followings may be used, but not limited thereto: propylene glycol, polyethylene glycol, vegetable oils such as olive oil, injectable esters such as ethyl oleate, etc.

The composition of the present invention may be administered orally or parenterally (e.g., intravenously, subcutaneously, intraperitoneally or topically) depending on the intended use. The dose of the pharmaceutical composition varies depending on the patient's weight, age, sex, health conditions, diet, the time of administration, the mode of administration, excretion rate, the severity of the disease, and the like.

A pharmaceutically effective dose and effective dosage of the pharmaceutical composition of the present invention may vary depending on a method for formulating the pharmaceutical composition, an administration mode, an administration time and/or administration route, etc., and may be diversified according to various factors including a type and degree of reaction to be achieved by means of administration of the pharmaceutical composition, a type of an individual for administration, such individual's age, weight, general health condition, disease symptom or severity, gender, diet and excretion, components of other drug compositions to be used for the corre-sponding individual at the same time or different times, etc., as well as other similar factors well known in a pharmaceutical field, and those skilled in the art may easily determine and prescribe an effective dosage for intended treatment.

The pharmaceutical composition of the present invention may be administered once a day or divided into several times a day. The pharmaceutical composition of the present invention may be administered as an individual therapeutic agent or in combination with other therapeutic agents, and may be administered sequentially or simultaneously with a conventional therapeutic agent. Considering all the factors above, the pharmaceutical composition of the present invention may be administered in such an amount that a maximum effect may be achieved by a minimum amount without a side effect, and such amount may be easily determined by those skilled in the art to which the present invention pertains.

In addition to the compound represented by Formula I, isomers thereof or pharmaceutically acceptable salts thereof, thepharmaceutical composition of the present invention may further contain at least one effective component which shows a medicinal effect the same thereas or similar thereto.

The pharmaceutical composition of the present invention may exhibit an excellent effect even when solely used, but may be further used in combination with various methods such as hormone therapy, drug treatment, etc. in order to increase a therapeutic efficiency.

The present invention also provides a method for preventing or treating CMT disease, comprising a step of administering a therapeutically effective amount of the compound represented by Formula I, isomers thereof or pharmaceutically acceptable salts thereof or the pharmaceutical composition comprising thereof, into a subject in need thereof.

As used herein, the term “therapeutically effective amount” refers to an amount of the compound represented by Formula I, isomers thereof or pharmaceutically acceptable salts thereof, which are effective in preventing or treating CMT disease.

In the therapeutic method of the present invention, a suitable total daily amount of the compound represented by Formula I, isomers thereof or pharmaceutically acceptable salts thereof may be determined within a range of correct medical decision by doctors in charge, and may be, for example, in a range of about 0.1 to 10,000 mg/kg, in a range of about 1 to 8,000 mg/kg, in a range of about 5 to 6,000 mg/kg, or in a range of about 10 to 4,000 mg/kg, and preferably the amount thereof in a range of about 50 to 2,000 mg/kg may be administered once a day or divided into several times a day. However, for the purpose of the present invention, it is preferable that a specific, therapeutically effective dose should be differently applied to each certain patient depending on various factors including a type and degree of reaction to be achieved therefrom, a specific composition including a presence of other preparations used in some cases, a patient's age, weight, general health condition, gender and diet, an administration time, an administration route, a secretion rate of the composition, a treatment period and a drug used together with the specific composition or simultaneously therewith, as well as other similar factors well known in a pharmaceutical field.

The method for preventing or treating CMT disease according to the present invention includes not only dealing with the disease itself before expression of its symptoms, but also inhibiting or avoiding such symptoms by administering the compound represented by Formula I, isomers thereof or pharmaceutically acceptable salts thereof. In managing the disease, a preventive or therapeutic dose of a certain active component may vary depending on a nature and severity of the disease or condition and a route of administering the active component. A dose and a frequency thereof may vary depending on an individual patient's age, weight and reactions. A suitable dose and usage may be easily selected by those skilled in the art, naturally considering such factors. Also, the method for preventing or treating CMT disease according to the present invention may further include administering a therapeutically effective amount of an additional active agent, which is helpful in treating the disease, along with the compound represented by Formula I, isomers thereof or pharmaceutically acceptable salts thereof, and the additional active agent may exhibit a synergy effect or an additive effect together with the compound represented by Formula I.

The present invention also provides a use of the compound represented by Formula I, isomers thereof or pharmaceutically acceptable salts thereof in preparation of a medicament for preventing or treating CMT disease. For the preparation of a medicament, the compound represented by Formula I, isomers thereof or pharmaceutically acceptable salts thereof may be combined with acceptable adjuvants, diluents, carriers, etc., and may be prepared into a complex preparation together with other active agents and thus have a synergy action of active ingredients.

The present invention also provides a use of the compound represented by formula I, isomers thereof or pharmaceutically acceptable salts thereof for preventing or treating Charcot-Marie-Tooth (CMT) diseases.

Matters mentioned in the use, composition and therapeutic method of the present invention are equally applied, if not contradictory to each other.

### Advantageous Effects of Invention

A pharmaceutical composition comprising a compound represented by Formula I according to the present invention, isomers thereof or pharmaceutically acceptable salts thereof selectively has histone deacetylase 6 (HDAC6) inhibitory activity and thus is effective in preventing or treating CMT disease.

### BRIEF DESCRIPTION OF DRAWINGS

FIG. 1 shows the results of evaluating behaviors and a degree of electrophysiological improvement after repeatedly administering a compound 84 into 2.5-week-old CMT1A mice (C22) for two weeks according to one embodiment of the present invention (N=10 per group. Normal group: WT dosed with DW, Control group: CMT littermate mice dosed with DW (CW 22). N=10), (a) Time of latency to fall, (b) Grip strength (measured in two weeks after administration), (c) MNCV, and (d) CMAP (measured in three weeks after administration). Data are indicated as mean±SEM. One-way ANOVA using Dunnett test was used to make a comparison between Tg and pharmacotherapeutic groups. *p<0.05, **p<0.01, ***p<0.0001. T-test was used to make a comparison between WT and Tg. #p<0.05, ###p<0.001.

FIG. 2 shows the results of evaluating a degree of histopathological improvement after repeatedly administering the compound 84 into 2.5-week-old CMT1A mice (C22) for two weeks according to one embodiment of the present invention (N=4 per group. Normal group: Wild type (WT) dosed with distilled water (DW), Control group: CMT littermate mice dosed with distilled water (DW), N=3), (a & b) Size distribution histogram of myelinated fibers and unmyelinated fibers, (c) Number of myelinated axons and unmyelinated axons, (d) Total number of axons, and (e) Representative histological image of toluidine blue-dyed sciatic nerve cross section for each group. Scale bar: 50 μm (×200). WT was dosed with DW, and C22 was dosed with DW or dosed with the compound 84 at 10 mg/kg, 25 mg/kg or 100 mg/kg for two weeks. Data are indicated as mean±SEM. One-way ANOVA using Dunnett test was used to make a comparison between control and pharmacotherapeutic groups. *p<0.05, **p<0.01, ***p<0.0001. T-test was used to make a comparison between normal (WT) and control (Tg) groups. #p<0.05, ###p<0.001.

### MODE FOR THE INVENTION

Hereinafter, the present invention will be described in more detail through preparation examples and embodiments. However, these preparation examples and embodiments are provided only for the purpose of illustrating the present invention, and thus the present invention is not limited thereto.

### Preparation Example 1: Synthesis of Compound 84 (4-(((3R,5S)-4-benzyl-3,5-dimethylpiperazin-1-yl)methyl)-N-hydroxybenzamide)

### Step 1: Synthesis of methyl 4-(((3R,5S)-3,5-dimethylpiperazin-1-yl)methyl)benzoate

(2S,6R)-2,6-dimethylpiperazine (50.000 g, 437.867 mmol) and Cs₂CO₃ (171.199 g, 525.440 mmol) were dissolved in acetonitrile (200 mL) at 0° C., and methyl 4-(bromomethyl)benzoate (formula 1-1, 80.242 g, 350.293 mmol) was added to the solution, followed by stirring at room temperature for 5 hours. The reaction mixture was filtered through a glass filter to remove solids, and the filtrate was concentrated under reduced pressure to remove the solvent. Water was added to the concentrate, followed by extraction with ethyl acetate. The organic layer was washed with a saturated aqueous solution of sodium chloride, dried with anhydrous magnesium sulfate, filtered, and concentrated under reduced pressure. Hexane (100 mL) was added to the concentrate and stirred, and the precipitated solid was filtered, washed with hexane, and dried to yield the desired compound (85.200 g, 74.2%) as a white solid.

### Step 2: Synthesis of methyl 4-(((3R,5S)-4-benzyl-3,5-dimethylpiperazin-1-yl)methyl)benzoate

Methyl 4-(((3R,5S)-3,5-dimethylpiperazin-1-yl)methyl)benzoate (formula 1-2, 30.000 g, 114.351 mmol), benzyl bromide (14.961 mL, 125.786 mmol) and K₂CO 3 (23.707 g, 171.527 mmol) were dissolved in acetonitrile (150 mL) at room temperature, and the solution was stirred at the same temperature for 17 hours. The reaction mixture was filtered through a glass filter to remove solids, and the filtrate was concentrated under reduced pressure to remove the solvent. Water was added to the concentrate, followed by extraction with ethyl acetate. The organic layer was washed with a saturated aqueous solution of sodium chloride, dried with anhydrous magnesium sulfate, filtered, and concentrated under reduced pressure. The concentrate was purified by column chromatography (silicon dioxide; 120 g cartridge; ethyl acetate/hexane=from 0% to 30%) and concentrated to afford the desired compound (22.400 g, 55.6%) as a white solid.

### Step 3: Synthesis of Compound 84

Methyl 4-(((3R,5S)-4-benzyl-3,5-dimethylpiperazin-1-yl)methyl)benzoate (formula 1-3, 15.000 g, 42.557 mmol), hydroxylamine (52.061 mL, 851.136 mmol, 50.00% aqueous solution) and potassium hydroxide (23.879 g, 425.568 mmol) were dissolved in methanol (300 mL) at 0° C., and the solution was stirred at the same temperature for 1 hour. The reaction mixture was concentrated under reduced pressure to remove the solvent, and a saturated aqueous solution of sodium hydrogen carbonate was added to the concentrate, followed by extraction with methylene chloride. The organic layer was washed with a saturated aqueous solution of sodium chloride, dried with anhydrous magnesium sulfate, filtered, and concentrated under reduced pressure. The concentrate was purified by column chromatography (silicon dioxide; 120 g cartridge; methanol/methylene chloride=from 0% to 20%) and concentrated, and then the obtained material was crystallized from diethyl ether (200 mL) and methylene chloride (50 mL) at 25° C. and filtered. The resulting solid was washed with diethyl ether and dried to yield compound 84 (12.580 g, 83.6%) as a white solid.

¹H NMR (400 MHz, DMSO-d₆) δ 11.18 (brs, 1H), 9.03 (brs, 1H), 7.70 (d, 2H, J=8.2 Hz), 7.36-7.33 (m, 4H), 7.28 (dd, 2H, J=7.5, 7.5 Hz), 7.18 (dd, 1H, J=7.2, 7.2 Hz), 3.73 (s, 2H), 3.44 (s, 2H), 2.64 (d, 2H, J=10.6 Hz), 2.61-2.53 (m, 2H), 1.81 (t, 2H, J=10.5 Hz), 0.90 (d, 6H, J=6.1 Hz); LRMS (ES) m/z 354.2 (M++1).

### Preparation Example 2: Synthesis of Compound 382 (4-(((3R,5S)-3,5-dimethyl-4-(3-(piperidin-1-ylmethyl)benzyl)piperazin-1-yl)methyl)—N-hydroxybenzamide)

### Step 1: Synthesis of methyl 4-(((3R,5S)-3,5-dimethyl-4-(3-(piperidin-1-ylmethyl)benzyl)piperazin-1-yl)methyl)ben zoate

Methyl 4-(((3R,5S)-4-(3-formylbenzyl)-3,5-dimethylpiperazin-1-yl)methyl)benzoate (formula 5-1, 0.150 g, 0.394 mmol) and piperidine (0.038 mL, 0.434 mmol) were dissolved in methylene chloride (10 mL), and the solution was stirred at room temperature for 30 minutes. Na(OAc)₃BH (0.125 g, 0.591 mmol) was added to the reaction solution, followed by stirring at the same temperature for 17 hours. Then, a saturated aqueous solution of sodium hydrogen carbonate was added to the reaction mixture, followed by extraction with methylene chloride. The extract was filtered through a plastic filter to remove solid residue and an aqueous layer, and then concentrated under reduced pressure. The concentrate was purified by column chromatography (silicon dioxide, 4 g cartridge; methanol/methylene chloride=from 0% to 10%) and concentrated to afford the desired compound (0.095 g, 53.6%) as a pale yellow oil.

### Step 2: Synthesis of Compound 382

Methyl 4-(((3R,5S)-3,5-dimethyl-4-(3-(piperidin-1-ylmethyl)benzyl)piperazin-1-yl)methyl)ben zoate (formula 5-2, 0.044 g, 0.098 mmol), hydroxylamine (0.060 mL, 0.979 mmol, 50.00% aqueous solution) and potassium hydroxide (0.127 g, 1.957 mmol) were dissolved in methanol (3 mL) at room temperature, and the solution was stirred at the same temperature for 30 minutes. Then, the reaction mixture was concentrated under reduced pressure to remove the solvent, and a saturated aqueous solution of sodium hydrogen carbonate was added to the concentrate, followed by extraction with methylene chloride. The extract was filtered through a plastic filter to remove solid residue and an aqueous layer, and then concentrated under reduced pressure to afford compound 382 (0.037 g, 83.9%) as a white solid.

¹H NMR (400 MHz, DMSO-d₆) δ 7.68 (d, 2H, J=8.2 Hz), 7.30-7.28 (m, 3H), 7.22-7.20 (m, 2H), 7.08 (d, 1H, J=6.2 Hz), 3.72 (s, 2H), 3.41 (s, 2H), 3.40 (s, 2H), 2.65-2.62 (m, 2H), 2.58-2.53 (m, 2H), 2.29 (s, 4H), 1.80 (t, 2H, J=10.7 Hz), 1.49-1.46 (m, 4H), 1.40-1.38 (m, 2H), 0.90 (d, 6H, J=6.1 Hz); LRMS (ES) m/z 451.2 (M++1).

### Preparation Example 3: Synthesis of Compound 454 (N-hydroxy-4-(((3R,5S)-4-(3-((4-isopentylpiperazin-1-yl)methyl)benzyl)-3,5-dimethylpiperazin-1-yl)methyl)benzamide)

### Step 1: Synthesis of tert-butyl 4-isopentylpiperazine-1-carboxylate

Tert-butyl piperazine-1-carboxylate (2.000 g, 10.738 mmol), 1-bromo-3-methylbutane (1.352 mL, 11.275 mmol) and Cs₂CO₃ (4.198 g, 12.886 mmol) were dissolved in acetonitrile (150 mL) at room temperature, and the solution was stirred at the same temperature for 17 hours. The reaction mixture was filtered through a glass filter to remove solids, and the filtrate was concentrated under reduced pressure to remove the solvent. The concentrate was purified by column chromatography (silicon dioxide, 12 g cartridge; ethyl acetate/hexane=from 0% to 10%) and concentrated to afford the desired compound (1.220 g, 44.3%) as a colorless oil.

### Step 2: Synthesis of 1-isopentylpiperazine trifluoroacetate

Tert-butyl 4-isopentylpiperazine-1-carboxylate (1.000 g, 3.900 mmol) was dissolved in methylene chloride (10 mL)/trifluoroacetic acid (5 mL) at room temperature, and the solution was stirred at the same temperature for 17 hours. The reaction mixture was concentrated under reduced pressure to remove the solvent, and the concentrate was crystallized from ethyl acetate (20 mL) at room temperature and filtered, and the resulting solid was washed with ethyl acetate and dried to afford the desired compound (0.929 g, 94.0%) as a white solid.

### Step 3: Synthesis of methyl 4-(((3R,5S)-4-(3-((4-isopentylpiperazin-1-yl)methyl)benzyl)-3,5-dimethylpiperazin-1-ylmethyl)benzoate

Methyl 4-(((3R,5S)-4-(3-formylbenzyl)-3,5-dimethylpiperazin-1-yl)methyl)benzoate (formula 5-1, 0.220 g, 0.578 mmol) and 1-isopentylpiperazine trifluoroacetate (0.220 g, 0.867 mmol) were dissolved in methylene chloride (5 mL), and the solution was stirred at room temperature for 1 hour. Na(OAc)₃BH (0.245 g, 1.156 mmol) was added to the reaction solution, which was then further stirred at the same temperature for 17 hours. Then, a saturated aqueous solution of sodium hydrogen carbonate was added to the reaction mixture, followed by extraction with methylene chloride. The extract was filtered through a plastic filter to remove solid residue and an aqueous layer, and then concentrated under reduced pressure. The concentrate was purified by column chromatography (silicon dioxide, 4 g cartridge; methanol/methylene chloride=from 0% to 10%) and concentrated to afford the desired compound (0.232 g, 77.0%) as a pale yellow oil.

### Step 4: Synthesis of Compound 454

Methyl 4-(((3R,5S)-4-(3-((4-isopentylpiperazin-1-yl)methyl)benzyl)-3,5-dimethylpiperazin-1-yl)methyl)benzoate (formula 5-2, 0.232 g, 0.446 mmol), hydroxylamine (0.545 mL, 8.910 mmol, 50.00% aqueous solution) and potassium hydroxide (0.250 g, 4.455 mmol) were dissolved in methanol (5 mL) at room temperature, and the solution was stirred at the same temperature for 30 minutes. The reaction mixture was concentrated under reduced pressure to remove the solvent, and a saturated aqueous solution of sodium hydrogen carbonate was added to the resulting concentrate, followed by extraction with methylene chloride. The extract was filtered through a plastic filter to remove solid residue and an aqueous layer, and was then concentrated under reduced pressure to afford compound 454 (0.067 g, 29.6%) as a white solid.

¹H-NMR (400 MHz, DMSO-d₆) δ 7.69 (d, 2H, J=8.2 Hz), 7.30 (d, 2H, J=8.1 Hz), 7.27 (s, 1H), 7.23-7.21 (m, 2H), 7.09-7.08 (m, 1H), 3.72 (s, 2H), 3.42 (s, 2H), 3.41 (s, 2H), 2.63 (d, 2H, J=10.2 Hz), 2.58-2.53 (m, 2H), 2.34-2.27 (m, 8H), 2.24 (t, 2H, J=7.6 Hz), 1.80 (t, 2H, J=10.5 Hz), 1.57-1.53 (m, 1H), 1.31-1.25 (m, 2H), 0.89 (d, 6H, J=6.1 Hz), 0.85 (d, 6H, J=6.6 Hz).

### <Example 1> Analysis of Effect of HDAC6-Specific Inhibitor on Axonal Transport Velocity of Mitochondria (In Vitro)

An effect of HDAC6-specific inhibitor on axonal transport of mitochondria was analyzed to identify if a compound represented by Formula I of the present invention selectively inhibits an HDAC6 activity and thus increases acetylation of tubulin, i.e., a key substrate of HDAC6 to show an effect of improving a transport velocity of mitochondria, which had been decreased by amyloid-beta treatment within a neuronal axon. To that end, on the 17^{th} to 18^{th} days (E17-18) of insemination, the hippocampal neurons from a Sprague-Dawley (SD) rat fetus were cultured for seven days in a culture container for imaging, which had been coated with extracellular matrix, and were treated with amyloid-beta protein fragments at a concentration of 1M. In 24 hours later, such neurons were treated with the compound on the 8^{th} day of in vitro culture. In three hours later, they were treated with MitoTracker Red CMXRos (Life Tech-nologies, NY, USA) for last five minutes to stain mitochondria. An image on the axonal transport of stained neuron mitochondria was taken with a confocal microscope (Leica SP8; Leica microsystems, UK) at an interval of one second for one minute to measure a transport velocity of each mitochondria per second with an IMARIS analysis program (BITPLANE, Zurich, Switzerland), and to set a section, in which the group treated with amyloid-beta had shown a significant decrease in the transport velocity of mitochondria compared to a vehicle. After that, the results of normalization with the vehicle set at 100% and with the group treated with amyloid-beta set at 0% were described in the following table 1.

TABLE 1

| | | |
|---|---|---|
| Compound | Concentration | Velocity distribution (%) |

| | | |
|---|---|---|
| Vehicle | — | 100% |
| Amyloid beta | — | 0% |

| | | | |
|---|---|---|---|
| 84 | 0.5 | uM | * |
| | 15 | uM | *** |
| 454 | 1 | uM | * |
| | 3 | uM | * |
| | 15 | uM | ** |

Upon treatment with the compound, a velocity distribution is indicated as:
* 0%~50%;
** 50%~100%;
*** >100%

From the results above, it was identified that the compound represented by Formula I of the present invention shows an excellent efficacy of improving an axonal transport velocity of mitochondria as shown in the table 1 above.

### <Example 2> HDAC6/1 Enzyme Inhibition Assay (In Vitro)

A comparative experiment was conducted with an existing developed material as a control group in order to identify the selectivity of the compound represented by Formula I of the present invention to HDAC6 through an experiment on HDAC1 and HDAC6 enzyme activity inhibition.

The HDAC enzyme activity was measured with HDAC Fluorimetric Drug Discovery Kit (BML-[1424] AK511, 516) of Enzo Life Science, Inc. For the test on the HDAC1 enzyme activity, human recombinant HDAC1 (BML-SE456) was used as an enzyme source and Fluor de Lys®-“SIRT1 (BNL-KI177)” was used as a substrate. A 5-fold dilution of the compound was divided into a 96-well plate, after which 0.3 μg of the enzyme and 10 μM of the substrate were inserted into each well and subjected to reaction at 30° C. for 60 minutes, such that Fluor de Lys® Developer II (BMLKI176) was inserted thereinto and subjected to reaction for 30 minutes and finished. After that, a fluorescence value (Ex 360, Em 460) was measured with a multi-plate reader (Flexstation 3, Molecular Device). An experiment on HDAC6 enzyme was conducted in accordance with the same protocol as an HDAC1 enzyme activity test method by using human recombinant HDAC6 (382180) of Calbiochem Inc. Each IC₅₀ value was calculated with GraphPad Prism 4.0 program, and final result values were described in the following table 2.

TABLE 2

| | HDAC1 | HDAC6 | HDAC6 |
|---|---|---|---|
| Compound | (μM) | (μM) | selectivity (fold) |
| 84 | 11.9 | 0.023 | 517 |
| 382 | >10 | 0.083 | >120 |
| 454 | ND | 0.044 | >237 |

As shown in the table 2 above, it was identified that the compounds represented by Formula I of the present invention show an excellent selective HDAC6 inhibitory activity.

### <Example 3> Analysis of HDAC6 Inhibitory Effect and Selectivity for Other HDACs

The HDAC6 inhibitory effect and selectivity of the compound represented by Formula I were identified at an enzyme level.

The experiment was requested at Reaction biology Corp. (Malvern, Pa., USA) and performed according to a test method established within the organization. Particularly, a serial dilution of the compound 84 was divided into a plate, after which a substrate, i.e., RHK—K(Ac)-AMC and an enzyme were inserted together into 50 mM Tris-HCl buffer (pH 8.0, 137 mM NaCl, 2.7 mM KCl, 1 mM MgCl₂, 1 mg/ml BSA) to induce a reaction. After that, 50 mm Tris-HCl buffer (pH 8.0, 137 mM NaCl, 2.7 mM KCl, 1 mM MgCl₂) containing 2 mM nicotinamide and 16 mg/mL trypsin was inserted thereinto and then subjected to reaction, after which a fluorescence signal was measured at Ex. 360 nm/Em. 460 nm to measure an enzyme activity, such that the results thereof were shown in the following table 3. Pan-HDAC inhibitor, i.e., LBH589 was used as a control group.

TABLE 3

| | IC₅₀ (nM) | | | | | | | | | | |
|---|---|---|---|---|---|---|---|---|---|---|---|
| | HDAC-1 | HDAC-2 | HDAC-3 | HDAC-4 | HDAC-5 | HDAC-6 | HDAC-7 | HDAC-8 | HDAC-9 | HDAC-10 | HDAC-11 |
| LBH589 | 3.8 | 12 | 4.8 | 120 | 6.3 | 3.2 | 12.7 | 53 | 8.6 | 8.7 | 7.8 |
| Compound | ND | ND | ND | ND | ND | 72.2 | ND | 5170 | ND | ND | ND |
| 84 | | | | | | | | | | | |

As shown in the table 3 above, it was identified that IC₅₀ for HDAC6 of the compound 84 is 72.2 nM and IC₅₀ for HDAC8 is 5170 nM, while other HDAC isotypes are not inhibited at all.

In other words, it was identified that the compound 84 is excellent in HDAC6 inhibitory capacity and very selective to other HDAC isotypes.

### <Example 4> Evaluation of Medicinal Effect

To identify a therapeutic effect and mechanism of the compound 84 on CMT disease, CMT1A mice (C22) were treated with the compound 84 to identify the animal's behavioral symptoms as well as histological improvements and changes in protein expression.

CMT1A occurs at the highest frequency in the group of unmyelinating CMT diseases caused by duplication of PMP22 genes which constitute myelin. It is known that the CMT1A mice (C22) used in the test have seven human PMP22 genes inserted therein, which are overexpressed about 2.7 times more than endogenous mouse PMP22 mRNA. Symptoms such as a decline in motor functions, abnormal sense, etc. occurred to the mice from before 2.5 weeks old, and muscle weakness, hoof, tail drag are observed therefrom due to degeneration of peripheral nerves. Histologically, a decrease in myelination, schwann cell hypertrophy, onion bulb, axonal degeneration/loss and muscular atrophy are observed.

Particularly, the compound 84 was repeatedly administered into the 2.5-week-old CMT1A mice (C22) at 10, 25 and 100 mg/kg twice a day for two weeks. Behavioral evaluation and electrophysiological analysis were performed on the last day, immediately after which the animals were sacrificed to carry out histopathological analysis and evaluation.

All the results were indicated as mean±standard error, and the validity of medicinal effect was determined through statistical significance between negative control group and each test material group. As for statistical analysis, the homogeneity of dispersion was identified with ANOVA (one-way ANOVA for single measurement and two-way ANOVA for repeated measurement). As a result of Dunnett or Bonferroni post test, it was determined as statistically significant, if p value is less than 0.05.

### <Example 4-1> Evaluation of Animal Behaviors

Rotarod, balance beam and grip strength tests were conducted once a day for two days. Thereafter, the animals were distributed by the Z-array method based on the results of rotarod, grip strength, and body weight. C22 mice were divided into four groups consisting of DW treated group and three compound 84 treated groups (10, 25, 100 mg/kg) with 10 mice per group. The test articles were repeatedly administered twice a day orally, and the behavioral test was performed 30 min after administration of the test articles.

Rotarod Test

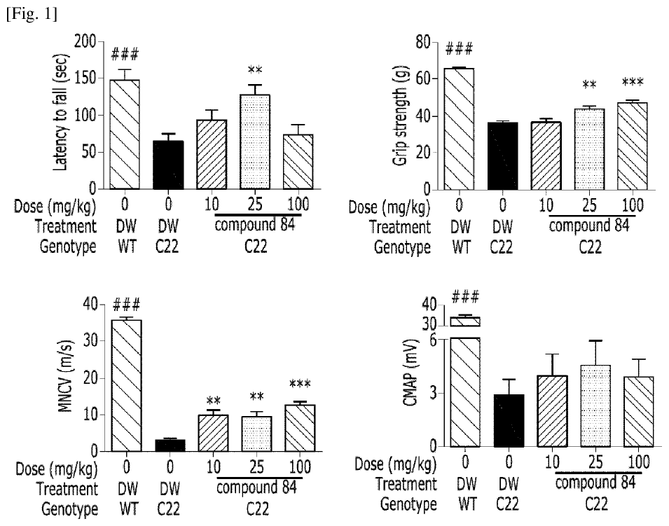

The Rotarod test (ROTA ROD, LE8205, Panlab) was performed to evaluate a motor coordination function/motor function. For three days before the test, all the test animals were placed on a road at 8 rpm and subjected to adaptive training five times a day. The animals, whose time of falling from the rod reached 150-180 seconds, were used in the test (including about 80% of all the animals). In both group separation and the present experiment, time at which animals fell from the rod was measured at a fixed speed of 8 rpm for three minutes. In a single experiment, the Rotarod test was performed three times in total. Out of its three results, the maximum value was used as an evaluation value. The results thereof were shown in FIG. 1(a).

In the FIG. 1, DW WT (littermate) means the normal group of mice dosed with distilled water, and DW C22 (littermate) means CMT mice (control group) dosed with distilled water.

As a result of repeatedly administering the compound 84 into the 2.5-week-old CMT1A mice (C22) for two weeks, it might be seen that time of latency to fall is increased in a dose-dependent manner compared to the control group (littermate dosed with DW (DW C22)), thus alleviating CMT disease, as shown in FIG. 1(a).

Grip Strength Test

CMT (hereditary motor and sensory neuropathy) shows a key symptom, in which the distribution of muscle-controlling nerves is decreased to cause muscle atrophy due to neurodegeneration. In an actual clinical test, a degree of muscle atrophy is evaluated with an ankle dorsiflexion examination and a grip test. GST (GRIP STRENGTH TEST, BIO-GS3, BIOSEB) is mainly used in an animal experiment. For the GST, strength of four feet was evaluated with grid. All the experiments were performed by one person. When a mouse tried a grip, a slightly little tension was applied there to make the mouse keep a grip, after which the mouse was pulled slightly above the horizon with a gradient of about 150 to measure the maximum tension. Out of the values consecutively measured five times, the maximum value was calibrated with a weight, and then used as evaluation results. The test was performed in the same way as above once a week for two weeks before and during drug administration. The results thereof were shown in FIG. 1(b).

As a result of repeatedly administering the compound 84 into the 2.5-week-old CMT1A mice (C22) for two weeks, it might be seen that grip strength is increased in a dose-dependent manner compared to the control group (littermate dosed with DW (DW C22)), as shown in FIG. 1(b).

Nerve Conduction Study (NCS)

The evaluation of the nerve conduction study (NSC) was as follows. A supramaximal stimulus was applied to proximal peripheral nerves, after which the compound muscle action potentials (CMAP) produced from distal muscles controlled by the nerves were recorded to measure a latent period, amplitude, nerve conduction velocity (NCV), duration, shape, area and the like, which were then compared with normal values and thus evaluated. For the nerve conduction study (NSC), an electrical stimulus was given to the sciatic nerves of the mice anesthetized with isoflurane, after which the electrical stimulus given to sural nerves was measured with VikingQuest (Natus Medical Inc.). The results thereof were shown in FIGS. 1(c) and (d).

As a result of repeatedly administering the compound 84 into the 2.5-week-old CMT1A mice (C22) for two weeks, it might be seen that motor nerve conduction velocity (MNCV) and compound muscle action potentials (CMAP) are increased in a dose-dependent way compared to the control group (littermate dosed with DW (DW C22)), as shown in FIGS. 1(c) and (d).

As such, the compound 84 improved the motor ability and the electrophysiological index, which had been deficient in the C22 mice. Consequently, it was identified that the compound 84 has a medicinal effect as a therapeutic agent for CMT disease.

### <Example 4-2> Histopathological Analysis

A histopathological analysis was performed as follows. In 0.5 hours after the last administration of the compound 84, the mice were euthanized through cervical dis-location, after which their sciatic nerves were collected therefrom. For a histopathological examination, the collected sciatic nerves were fixed into 2.5% glu-taraldehyde solution. The fixed tissues were prepared into a plastic-embedded block through a tissue treatment process, then micro-sectioned in the semithin thickness of 500 nm to prepare sciatic nerve tissue fragments, and then stained with toluidine blue.

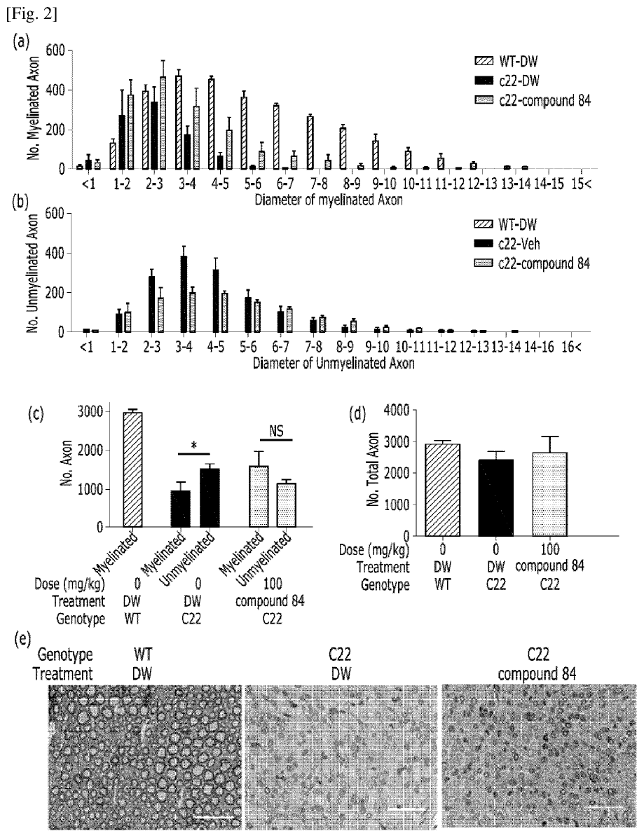

The stained nerve tissues were photographed at 400× magnification with an optical microscope. In a photographed entire area, the inner axon diameters of total myelinated fibers and total unmyelinated fibers were measured with an image J program. The results thereof were analyzed by using the numbers of total myelinated fibers and total unmyelinated fibers as well as a size distribution chart on the diameters of respective nerve fibers. The results thereof were shown in FIG. 2.

In the FIG. 2, DW WT (littermate) or WT-Veh (littermate) means the normal group of mice dosed with distilled water, and DW C22 (littermate) or C22-Veh (littermate) means CMT mice dosed with distilled water.

As shown in FIG. 2(e), it was identified in the histopathological evaluation of sciatic nerves that the control group of mice (C22, DW) shows a moderate loss of myelinated nerve fibers, axonal atrophy and schwann cell hyperplasia compared to the normal group of mice (WT, DW), while the animals dosed with the compound 84 shows a significant improvement in morphological changes (the loss of myelinated nerve fibers, axonal atrophy and schwann cell hyperplasia), which had appeared in the control group of mice.

In particular, as a result of quantifying a ratio of myelinated nerves and unmyelinated nerves, it was identified that the group dosed with the compound 84 shows a significant increase in the quantified ratio compared to the control group of mice (FIGS. 2(c) and (d)). As a result of analyzing a size distribution, it was seen that the control group (C22-Veh) shows a decrease in the size of diameters of myelinated nerves compared to the normal group (WT-Veh), while the group dosed with the compound 84 shows an increase in the size of diameters of myelinated nerves compared to the control group (FIG. 2(a)).

As such, it may be seen that the compound 84 alleviates the axonal atrophy and the loss of myelinated nerves, which have been observed in the CMT mice (C22). A therapeutic effect of the compound 84 on CMT disease might be identified from the present test.

1. A pharmaceutical composition for preventing or treating Charcot-Marie-Tooth (CMT) disease, comprising a compound represented by the following Formula I, isomers thereof or pharmaceutically acceptable salts thereof as an active ingredient: 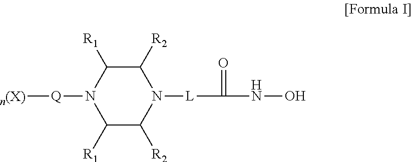
wherein
R₁ is hydrogen or —CH₃,
R₂ is hydrogen or —CH₃, wherein R₂ is —CH₃ when R₁ is hydrogen, and
R₂ is hydrogen when R₁ is —CH₃,
L is —(C₄-C₅ alkyl)-; —(C₁-C₃ alkyl)-L₁-; —C(═O)-L₁- or —S(═O)₂-L₁-,
wherein —(C₄-C₅ alkyl)- and —(C₁-C₃ alkyl)- may be unsubstituted or substituted with —CH₃,
L₁ is —(C₃-C₆)cycloalkyl-; 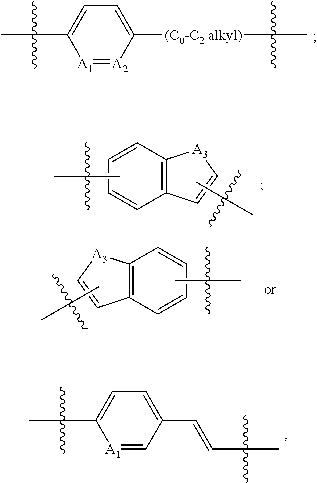
A₁ and A₂ are each independently —N— or —CR₃—, wherein both A₁ and A₂ cannot be —N—,
R₃ is hydrogen; —F; —Cl; —Br; —I; or —OH, and
A₃ is —NH— or —O—,
Q is selected from the group consisting of —(C₁-C₆)alkyl-; —(C₂-C₆)alkenyl-; —C(═O)—; —C(═S)—; —S(═O)₂— and 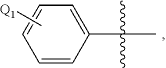
wherein —(C₁-C₆)alkyl- and —(C₂-C₆)alkenyl- may be unsubstituted or each independently substituted 1 to 3 —CH₃ groups or halogen atoms,
Q₁ is hydrogen; —F; —Cl; —Br; or —I,
n is an integer of 0, 1 or 2, wherein n is 0 when Q is 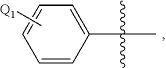
n is
1 when Q is —C(═O)—; —C(═S)— or —S(═O)₂-, and n is 1 or 2 when Q is —(C₁-C₆)alkyl- or —(C₂-C₆)alkenyl-, and
X may be selected from the group consisting of —C₁-C₆ alkyl; —C₃-C₆ cycloalkyl; —C₂-C₆ alkenyl; —C₃-C₆ cycloalkenyl; —(C₀-C₂ alkyl)Ar; —OAr; —(C₀-C₂ alkyl)Het; naphthyl and following groups: 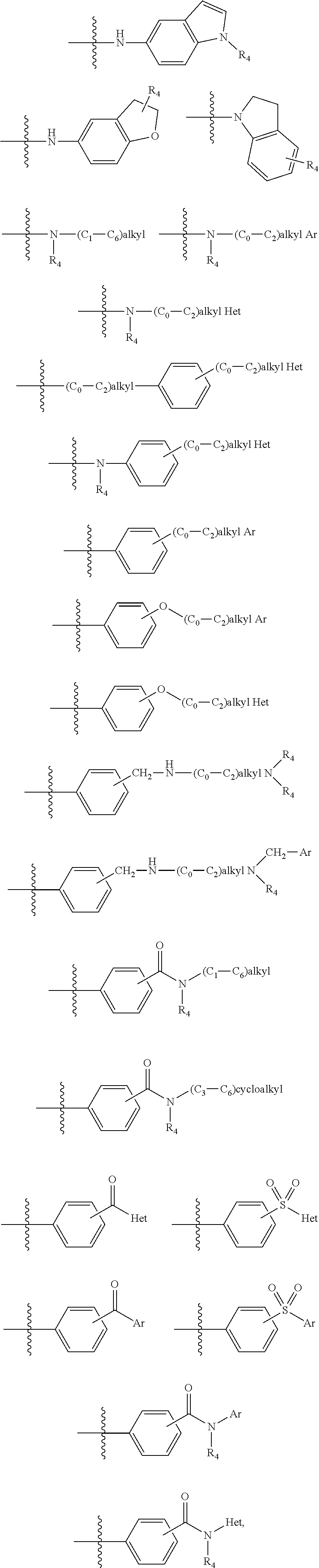
wherein R₄ is H or —C₁-C₄ alkyl,
—C₀-C₂ alkyl, —C₂-C₆ alkenyl and —C₁-C₆ alkyl may be unsubstituted or substituted with 1 to 2 —CH₃ groups; 1 to 3 —F groups; or both,
Ar is a C₆ monocyclic aromatic compound, which may be unsubstituted or substituted with one or more halogen atoms; —OH; —NH₂; —C₁—C₆ alkyl; —O(C₁-C₆)alkyl; —C₃-C₆ cycloalkenyl; —NH(C₁-C₆ alkyl); —N(C₁-C₃ alkyl)₂; —CH₂N(C₁-C₃ alkyl)₂; —S(═O)₂—(C₁-C₃ alkyl) or phenyl groups, wherein —C₁-C₃ alkyl; —C₁-C₆ alkyl and —C₃-C₆ cycloalkenyl may be each independently substituted with 1 to 5 —F or —CH₃ groups, and
Het is a 4- to 6-membered heteroaromatic or non-aromatic ring compound containing 1 to 3 elements selected from the group consisting of N, O and S while having 0 to 3 double bonds, and may be unsubstituted or substituted with one or more halogen atoms; —C₁-C₆ alkyl; —C(═O)(C₁-C₃ alkyl); —S(═O)₂ (C₁-C₃ alkyl) or benzyl groups, wherein —C₁-C₃ alkyl and —C₁-C₆ alkyl may be each independently substituted with —OH; 1 to 5 —F or —CH₃ groups.

2. The pharmaceutical composition according to claim 1, wherein the compound represented by Formula I is a compound represented by the following Formula II or Formula III: 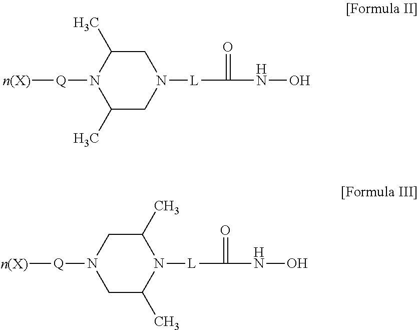
wherein
L is —(C₅ alkyl)-; —(C₁-C₂ alkyl)-L₁-; —C(═O)-L₁- or —S(═O)₂-L₁-,
wherein —(C₅ alkyl)- and —(C₁-C₂ alkyl)- are straight-chain and may be unsubstituted or substituted with —CH₃,
L₁ is —(C₃-C₆) cycloalkyl-; 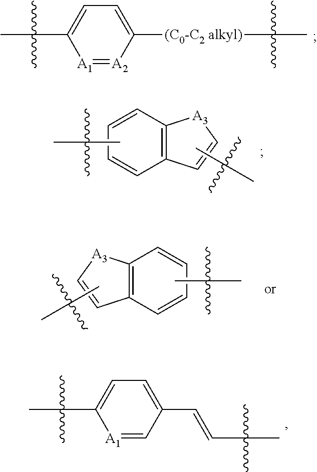
A₁ and A₂ are each independently —N— or —CR₃—, wherein both A₁ and A₂ cannot be —N—,
R₃ is hydrogen; —F or —OH, and
A₃ is —NH— or —O—,
Q is selected from the group consisting of —(C₁-C₃)alkyl-; —C(═O)—; —C(═S)—; —S(═O)₂— and Q, 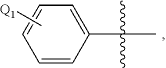
wherein —(C₁-C₃)alkyl- may be unsubstituted or substituted with 1 to 3 —CH₃ groups or halogen atoms,
Q₁ is hydrogen; —F or —Cl,
n is an integer of 0 or 1, wherein n is 0 when Q is 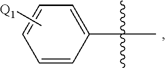
and
n is 1 when Q is —C(═O)—, —C(═S)—, —S(═O)₂— or —(C₁-C₃)alkyl-, and
X may be selected from the group consisting of —C₁-C₆ alkyl; —C₃-C₆ cycloalkyl; —C₂-C₆ alkenyl; —C₃-C₆ cycloalkenyl; —(C₀-C₂ alkyl)Ar; —OAr; —(C₀-C₂ alkyl)Het; naphthyl; and following groups: 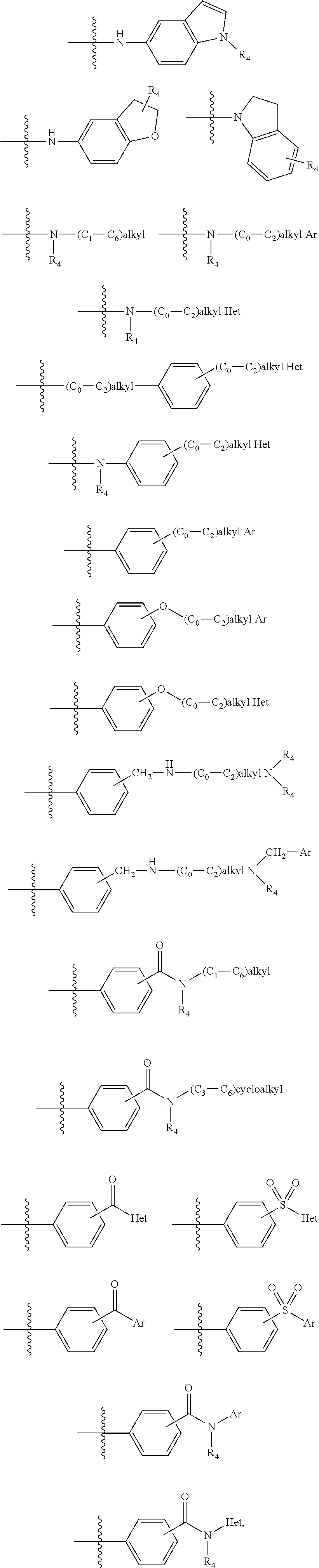
wherein R₄ is H or —C₁-C₄ alkyl, —C₀-C₂ alkyl; —C₂-C₆ alkenyl and —C₁-C₆ alkyl may be unsubstituted or substituted with 1 or 2 —CH₃ groups or 1 to 3 —F groups,
Ar is a C₆ monocyclic aromatic compound, which may be unsubstituted or substituted with one or more halogen atoms; —OH; —NH₂; —C₁—C₆ alkyl; —O(C₁-C₆)alkyl; —C₃-C₆ cycloalkenyl; —NH(C₁-C₆ alkyl); —N(C₁—C₃ alkyl)₂; —CH₂N(C₁-C₃ alkyl)₂; —S(═O)₂—(C₁-C₃ alkyl) or phenyl groups, wherein —C₁-C₃ alkyl; —C₁-C₆ alkyl and —C₃-C₆ cycloalkenyl may be each independently substituted with 1 to 5 —F or —CH₃ groups, and
Het is a 4- to 6-membered heteroaromatic or non-aromatic ring compound containing 1 to 3 elements selected from the group consisting of N; O and S while having 0 to 3 double bonds, and may be unsubstituted or substituted with one or more halogen atoms; —C₁-C₆ alkyl; —C(═O)(C₁-C₃ alkyl); —S(═O)₂ (C₁-C₃ alkyl) or benzyl groups, wherein —C₁-C₃ alkyl and —C₁-C₆ alkyl may be each independently substituted with —OH; or 1 to 5 —F or —CH₃ groups.

3. The pharmaceutical composition according to claim 1, wherein the Het is selected from the following group: 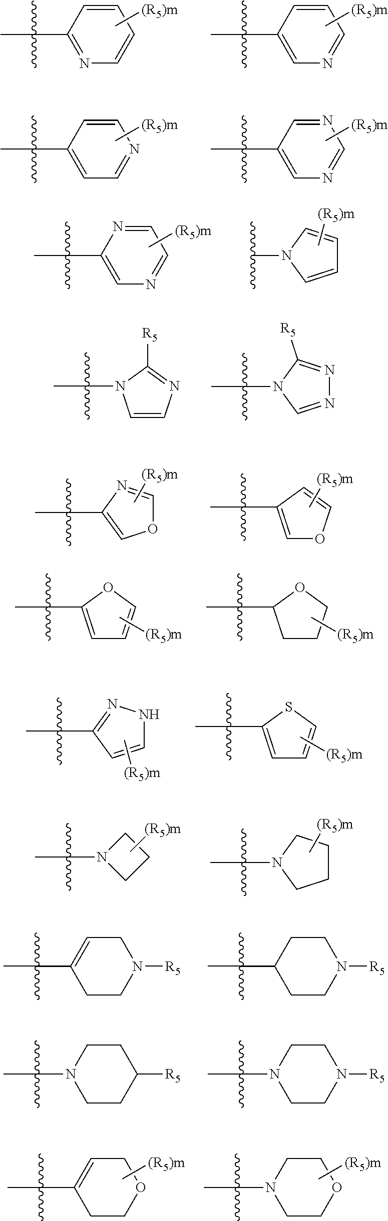
wherein, R₅ is each independently hydrogen; —F; —Cl; —C₁-C₆ alkyl; —C(═O)(C₁-C₃ alkyl); —S(═O)₂ (C₁-C₃ alkyl) or benzyl, wherein —C₁-C₃ alkyl and —C₁-C₆ alkyl may be each independently substituted with —OH; 1 to 5 —F or —CH₃, and
m is 0 or an integer of 1, 2 or 3 (when m is 0, the Het is unsubstituted, and when m is 1, 2 or 3, the Het may be each independently substituted with the R₅).

4. The pharmaceutical composition according to claim 1, wherein the L, L₁, A₁, A₂, A₃, R₃, R₄, Q and X have the following structure:
L is —CH₂-L₁-,
wherein
L₁ is 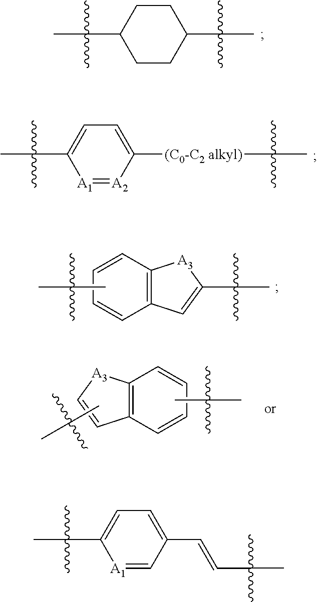
A₁ and A₂ are each independently —N— or —CR₃—, wherein both A₁ and A₂ cannot be —N—,
R₃ is hydrogen; —F or —OH, and
A₃ is —NH— or —O—,
Q is —CH₂—, —C(═O)— or —S(═O)₂—, and
X may be selected from the group consisting of —C₁-C₆ alkyl; —(C₀-C₂ alkyl)Ar; —(C₀-C₂alkyl)Het; —OAr; and following groups: 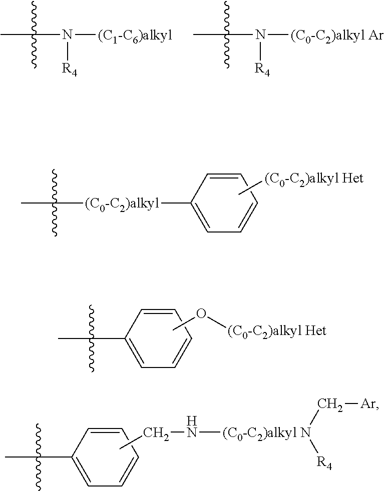
wherein R₄ is H or —C₁-C₄ alkyl,
—C₀-C₂ alkyl and —C₁-C₆ alkyl may be unsubstituted, substituted with 1 or 2 —CH₃ groups or 1 to 3 —F groups, or substituted with 1 or 2 —CH₃ groups and 1 to 3 —F groups, and
Ar and Het is each independently as defined in formula I.

5. The pharmaceutical composition according to claim 1, wherein the compound represented by Formula I is one of the compounds described in the following table:

| No. | Name of Compound |
|---|---|
| 080 | 4-(((2S,6R)-4-benzoyl-2,6-dimethylpiperazin-1- |
| | yl)methyl)-N-hydroxybenzamide |
| 081 | 4-(((2S,6R)-4-benzyl-2,6-dimethylpiperazin-1- |
| | yl)methyl)-N-hydroxybenzamide |
| 082 | 4-(((3R,5S)-4-acetyl-3,5-dimethylpiperazin-1- |
| | yl)methyl)-N-hydroxybenzamide |
| 083 | 4-(((3R,5S)-4-benzoyl-3,5-dimethylpiperazin-1- |
| | yl)methyl)-N-hydroxybenzamide |
| 084 | 4-(((3R,5S)-4-benzyl-3,5-dimethylpiperazin-1- |
| | yl)methyl)-N-hydroxybenzamide |
| 098 | (2S,6R)-4-(4-(hydroxycarbamoyl)benzyl)-N-isopropyl- |
| | 2,6-dimethylpiperazine-1-carboxamide |
| 099 | (2S,6R)-N-(3-chlorophenyl)-4-(4- |
| | (hydroxycarbamoyl)benzyl)-2,6-dimethylpiperazine-1- |
| | carboxamide |
| 100 | 4-(((3R,5S)-3,5-dimethyl-4-(phenylsulfonyl)piperazin- |
| | 1-yl)methyl)-N-hydroxybenzamide |
| 103 | (3R,5S)-4-(4-(hydroxycarbamoyl)benzyl)-N-isopropyl- |
| | 3,5-dimethylpiperazine-1-carboxamide |
| 104 | (3R,5S)-N-(3-chlorophenyl)-4-(4- |
| | (hydroxycarbamoyl)benzyl)-3,5-dimethylpiperazine-1- |
| | carboxamide |
| 105 | 4-(((2S,6R)-2,6-dimethyl-4-(phenylsulfonyl)piperazin- |
| | 1-yl)methyl)-N-hydroxybenzamide |
| 106 | 4-(((2S,6R)-4-(4-chlorobenzoyl)-2,6- |
| | dimethylpiperazin-1-yl)methyl)-N-hydroxybenzamide |
| 107 | 4-(((2S,6R)-4-(3-chlorobenzoyl)-2,6- |
| | dimethylpiperazin-1-yl)methyl)-N-hydroxybenzamide |
| 108 | 4-(((2S,6R)-4-(2-chlorobenzoyl)-2,6- |
| | dimethylpiperazin-1-yl)methyl)-N-hydroxybenzamide |
| 109 | 4-(((2S,6R)-4-((4-chlorophenyl)sulfonyl)-2,6- |
| | dimethylpiperazin-1-yl)methyl)-N-hydroxybenzamide |
| 110 | 4-(((2S,6R)-4-((2-chlorophenyl)sulfonyl)-2,6- |
| | dimethylpiperazin-1-yl)methyl)-N-hydroxybenzamide |
| 111 | 4-(((2S,6R)-2,6-dimethyl-4-picolinoylpiperazin-1- |
| | yl)methyl)-N-hydroxybenzamide |
| 112 | 4-(((2S,6R)-2,6-dimethyl-4-nicotinoylpiperazin-1- |
| | yl)methyl)-N-hydroxybenzamide |
| 113 | 4-(((2S,6R)-2,6-dimethyl-4-(pyridin-3- |
| | ylsulfonyl)piperazin-1-yl)methyl)-N-hydroxybenzamide |
| 114 | 4-(((2S,6R)-2,6-dimethyl-4-(thiophene-2- |
| | carbonyl)piperazin-1-yl)methyl)-N-hydroxybenzamide |
| 115 | 4-(((2S,6R)-4-(furan-2-carbonyl)-2,6- |
| | dimethylpiperazin-1-yl)methyl)-N-hydroxybenzamide |
| 118 | 4-(((2S,6R)-4-(2-chlorobenzyl)-2,6-dimethylpiperazin- |
| | 1-yl)methyl)-N-hydroxybenzamide |
| 119 | 4-(((2R,6S)-4-(4-fluorophenyl)-2,6-dimethylpiperazin- |
| | 1-yl)methyl)-N-hydroxybenzamide |
| 120 | 4-(((3R,5S)-4-(2-chlorobenzoyl)-3,5- |
| | dimethylpiperazin-1-yl)methyl)-N-hydroxybenzamide |
| 121 | 4-(((3R,5S)-4-(3-chlorobenzoyl)-3,5- |
| | dimethylpiperazin-1-yl)methyl)-N-hydroxybenzamide |
| 122 | 4-(((3R,5S)-4-(4-chlorobenzoyl)-3,5- |
| | dimethylpiperazin-1-yl)methyl)-N-hydroxybenzamide |
| 123 | 4-(((3R,5S)-3,5-dimethyl-4-picolinoylpiperazin-1- |
| | yl)methyl)-N-hydroxybenzamide |
| 125 | 4-(((3R,5S)-4-(furan-2-carbonyl)-3,5- |
| | dimethylpiperazin-1-yl)methyl)-N-hydroxybenzamide |
| 126 | 4-(((3R,5S)-3,5-dimethyl-4-(thiophene-2- |
| | carbonyl)piperazin-1-yl)methyl)-N-hydroxybenzamide |
| 127 | 4-(((3R,5S)-4-(2-chlorobenzyl)-3,5-dimethylpiperazin- |
| | 1-yl)methyl)-N-hydroxybenzamide |
| 128 | 4-(((3R,5S)-4-(3-chlorobenzyl)-3,5-dimethylpiperazin- |
| | 1-yl)methyl)-N-hydroxybenzamide |
| 145 | 4-(((3R,5S)-4-(furan-2-ylmethyl)-3,5- |
| | dimethylpiperazin-1-yl)methyl)-N-hydroxybenzamide |
| 146 | 4-(((3R,5S)-3,5-dimethyl-4-(2-phenylacetyl)piperazin- |
| | 1-yl)methyl)-N-hydroxybenzamide |
| 147 | 4-(((3R,5S)-4-ethyl-3,5-dimethylpiperazin-1- |
| | yl)methyl)-N-hydroxybenzamide |
| 148 | 4-(((3R,5S)-3,5-dimethyl-4-propylpiperazin-1- |
| | yl)methyl)-N-hydroxybenzamide |
| 149 | 4-(((3R,5S)-3,5-dimethyl-4-(2,2,2- |
| | trifluoroethyl)piperazin-1-yl)methyl)-N- |
| | hydroxybenzamide |
| 154 | 6-((3R,5S)-4-benzyl-3,5-dimethylpiperazin-1-yl)-N- |
| | hydroxyhexanamide |
| 159 | (2S,6R)-4-(4-(hydroxycarbamoyl)benzyl)-2,6-dimethyl- |
| | N-phenylpiperazine-1-carboxamide |
| 160 | (2S,6R)-4-(4-(hydroxycarbamoyl)benzyl)-2,6-dimethyl- |
| | N-(o-tolyl)piperazine-1-carboxamide |
| 161 | (2S,6R)-N-benzyl-4-(4-(hydroxycarbamoyl)benzyl)-2,6- |
| | dimethylpiperazine-1-carboxamide |
| 162 | (3R,5S)-4-(4-(hydroxycarbamoyl)benzyl)-3,5-dimethyl- |
| | N-phenylpiperazine-1-carboxamide |
| 163 | (3R,5S)-4-(4-(hydroxycarbamoyl)benzyl)-N-(2- |
| | methoxyphenyl)-3,5-dimethylpiperazine-1-carboxamide |
| 164 | (3R,5S)-4-(4-(hydroxycarbamoyl)benzyl)-N-(3- |
| | methoxyphenyl)-3,5-dimethylpiperazine-1-carboxamide |
| 165 | (3R,5S)-4-(4-(hydroxycarbamoyl)benzyl)-N-(4- |
| | methoxyphenyl)-3,5-dimethylpiperazine-1-carboxamide |
| 166 | (3R,5S)-4-(4-(hydroxycarbamoyl)benzyl)-3,5-dimethyl- |
| | N-(o-tolyl)piperazine-1-carboxamide |
| 167 | (3R,5S)-4-(4-(hydroxycarbamoyl)benzyl)-3,5-dimethyl- |
| | N-(m-tolyl)piperazine-1-carboxamide |
| 168 | (3R,5S)-N-benzyl-4-(4-(hydroxycarbamoyl)benzyl)-3,5- |
| | dimethylpiperazine-1-carboxamide |
| 171 | 4-(((3R,5S)-4-benzyl-3,5-dimethylpiperazin-1- |
| | yl)methyl)-2-fluoro-N-hydroxybenzamide |
| 172 | (E)-3-(4-(((3R,5S)-4-benzyl-3,5-dimethylpiperazin-1- |
| | yl)methyl)phenyl)-N-hydroxyacrylamide |
| 173 | 2-(4-(((3R,5S)-4-benzyl-3,5-dimethylpiperazin-1- |
| | yl)methyl)phenyl)-N-hydroxyacetamide |
| 174 | 4-(((2S,6R)-4-benzyl-2,6-dimethylpiperazin-1- |
| | yl)methyl)-2-fluoro-N-hydroxybenzamide |
| 175 | (E)-3-(4-(((2S,6R)-4-benzyl-2,6-dimethylpiperazin-1- |
| | yl)methyl)phenyl)-N-hydroxyacrylamide |
| 176 | 2-(4-(((2S,6R)-4-benzyl-2,6-dimethylpiperazin-1- |
| | yl)methyl)phenyl)-N-hydroxyacetamide |
| 177 | phenyl (2S,6R)-4-(4-(hydroxycarbamoyl)benzyl)-2,6- |
| | dimethylpiperazine-1-carboxylate |
| 183 | 4-(((2S,6R)-2,6-dimethyl-4-phenethylpiperazin-1- |
| | yl)methyl)-N-hydroxybenzamide |
| 184 | 4-(((3R,5S)-3,5-dimethyl-4-phenethylpiperazin-1- |
| | yl)methyl)-N-hydroxybenzamide |
| 185 | (3R,5S)-4-(4-(hydroxycarbamoyl)benzyl)-3,5-dimethyl- |
| | N-(1-methyl-1H-indol-5-yl)piperazine-1-carboxamide |
| 186 | (3R,5S)-N-(3-(1H-pyrrol-1-yl)phenyl)-4-(4- |
| | (hydroxycarbamoyl)benzyl)-3,5-dimethylpiperazine-1- |
| | carboxamide |
| 187 | (3R,5S)-N-(2,3-dihydrobenzofuran-5-yl)-4-(4- |
| | (hydroxycarbamoyl)benzyl)-3,5-dimethylpiperazine-1- |
| | carboxamide |
| 188 | 4-(((3R,5S)-3,5-dimethyl-4-(3- |
| | phenylpropanoyl)piperazin-1-yl)methyl)-N- |
| | hydroxybenzamide |
| 189 | 4-(((2S,6R)-2,6-dimethyl-4-(m- |
| | tolylcarbamothioyl)piperazin-1-yl)methyl)-N- |
| | hydroxybenzamide |
| 193 | 4-(((2S,6R)-4-(2-fluoro-2-methylpropyl)-2,6- |
| | dimethylpiperazin-1-yl)methyl)-N-hydroxybenzamide |
| 194 | 6-(((3R,5S)-4-benzyl-3,5-dimethylpiperazin-1- |
| | yl)methyl)-N-hydroxynicotinamide |
| 195 | 6-(((2S,6R)-4-benzyl-2,6-dimethylpiperazin-1- |
| | yl)methyl)-N-hydroxynicotinamide |
| 196 | (3R,5S)-4-(4-(hydroxycarbamoyl)benzyl)-N,3,5- |
| | trimethyl-N-phenylpiperazine-1-carboxamide |
| 197 | N-hydroxy-4-(((2S,6R)-4-(indoline-1-carbonyl)-2,6- |
| | dimethylpiperazin-1-yl)methyl)benzamide |
| 198 | (3R,5S)-N-butyl-4-(4-(hydroxycarbamoyl)benzyl)-3,5- |
| | dimethyl-N-phenylpiperazine-1-carboxamide |
| 204 | 4-(((3R,5S)-3,5-dimethyl-4-(4,4,4- |
| | trifluorobutyl)piperazin-1-yl)methyl)-N- |
| | hydroxybenzamide |
| 211 | 4-(((3R,5S)-4-(2-fluorobenzyl)-3,5-dimethylpiperazin- |
| | 1-yl)methyl)-N-hydroxybenzamide |
| 212 | 4-(((3R,5S)-4-(2,5-difluorobenzyl)-3,5- |
| | dimethylpiperazin-1-yl)methyl)-N-hydroxybenzamide |
| 213 | 4-(((3R,5S)-3,5-dimethyl-4-(2,4,5- |
| | trifluorobenzyl)piperazin-1-yl)methyl)-N- |
| | hydroxybenzamide |
| 214 | 4-(((3R,5S)-4-(3,5-bis(trifluoromethyl)benzyl)-3,5- |
| | dimethylpiperazin-1-yl)methyl)-N-hydroxybenzamide |
| 215 | 4-(((2S,6R)-2,6-dimethyl-4-(2,4,5- |
| | trifluorobenzyl)piperazin-1-yl)methyl)-N- |
| | hydroxybenzamide |
| 218 | 4-(2-((3R,5S)-4-benzyl-3,5-dimethylpiperazin-1- |
| | yl)ethyl)-N-hydroxybenzamide |
| 219 | 4-(1-((3R,5S)-4-benzyl-3,5-dimethylpiperazin-1- |
| | yl)ethyl)-N-hydroxybenzamide |
| 220 | 4-(((2S,6R)-2,6-dimethyl-4-(1-phenylethyl)piperazin- |
| | 1-yl)methyl)-N-hydroxybenzamide |
| 222 | 4-(((3R,5S)-4-(2-(3-fluorophenyl)acetyl)-3,5- |
| | dimethylpiperazin-1-yl)methyl)-N-hydroxybenzamide |
| 223 | 4-(((3R,5S)-3,5-dimethyl-4-(2-(3- |
| | (trifluoromethyl)phenyl)acetyl)piperazin-1- |
| | yl)methyl)-N-hydroxybenzamide |
| 224 | 4-(((3R,5S)-4-(2-(3-chlorophenyl)acetyl)-3,5- |
| | dimethylpiperazin-1-yl)methyl)-N-hydroxybenzamide |
| 225 | 4-(((3R,5S)-4-(3-fluorobenzyl)-3,5-dimethylpiperazin- |
| | 1-yl)methyl)-N-hydroxybenzamide |
| 230 | (3R,5S)-4-(4-(hydroxycarbamoyl)benzyl)-N-(3- |
| | methoxybenzyl)-3,5-dimethylpiperazine-1-carboxamide |
| | (Trifluoroacetic acid salt) |
| 231 | (3R,5S)-N-(3-fluorobenzyl)-4-(4- |
| | (hydroxycarbamoyl)benzyl)-3,5-dimethylpiperazine-1- |
| | carboxamide |
| 232 | (2S,6R)-N-(3-fluorobenzyl)-4-(4- |
| | (hydroxycarbamoyl)benzyl)-2,6-dimethylpiperazine-1- |
| | carboxamide |
| 233 | 4-(((2S,6R)-4-(2-(3-chlorophenyl)acetyl)-2,6- |
| | dimethylpiperazin-1-yl)methyl)-N-hydroxybenzamide |
| 234 | 4-(((3R,5S)-4-(2-([1,1′-biphenyl]-3-yl)acetyl)-3,5- |
| | dimethylpiperazin-1-yl)methyl)-N-hydroxybenzamide |
| 242 | 4-(((3R,5S)-4-(3-(furan-2-yl)benzoyl)-3,5- |
| | dimethylpiperazin-1-yl)methyl)-N-hydroxybenzamide |
| 243 | 4-(((3R,5S)-4-(3-(furan-3-yl)benzoyl)-3,5- |
| | dimethylpiperazin-1-yl)methyl)-N-hydroxybenzamide |
| 244 | 4-(((3R,5S)-4-(3-(3,6-dihydro-2H-pyran-4-yl)benzoyl)- |
| | 3,5-dimethylpiperazin-1-yl)methyl)-N-hydroxybenzamide |
| 245 | 4-(((3R,5S)-3,5-dimethyl-4-(3-(pyridin-4- |
| | yl)benzoyl)piperazin-1-yl)methyl)-N-hydroxybenzamide |
| | (Trifluoroacetic acid salt) |
| 246 | 4-(((3R,5S)-3,5-dimethyl-4-(3-(pyridin-3- |
| | yl)benzoyl)piperazin-1-yl)methyl)-N-hydroxybenzamide |
| 247 | 4-(((3R,5S)-4-(4′,4′-dimethyl-2′,3′,4′,5′-tetrahydro- |
| | [1,1′-biphenyl]-3-carbonyl)-3,5-dimethylpiperazin-1- |
| | yl)methyl)-N-hydroxybenzamide |
| 248 | 4-(((3R,5R)-4-benzyl-3,5-dimethylpiperazin-1- |
| | yl)methyl)-N-hydroxybenzamide |
| 249 | 4-(((3R,5R)-4-(furan-2-carbonyl)-3,5- |
| | dimethylpiperazin-1-yl)methyl)-N-hydroxybenzamide |
| 250 | 4-(((2S,6R)-4-(2-([1,1′-biphenyl]-3-yl)acetyl)-2,6- |
| | dimethylpiperazin-1-yl)methyl)-N-hydroxybenzamide |
| | (Trifluoroacetic acid salt) |
| 251 | 4-(((2S,6R)-2,6-dimethyl-4-(2-(3-(pyridin-4- |
| | yl)phenyl)acetyl)piperazin-1-yl)methyl)-N- |
| | hydroxybenzamide (Trifluoroacetic acid salt) |
| 252 | 4-(((2S,6R)-4-(2-(3-(3,6-dihydro-2H-pyran-4- |
| | yl)phenyl)acetyl)-2,6-dimethylpiperazin-1-yl)methyl)- |
| | N-hydroxybenzamide |
| 253 | 4-(((2S,6R)-4-(2-(4′,4′-dimethyl-2′,3′,4′,5′- |
| | tetrahydro-[1,1′-biphenyl]-3-yl)acetyl)-2,6- |
| | dimethylpiperazin-1-yl)methyl)-N-hydroxybenzamide |
| | (Trifluoroacetic acid salt) |
| 255 | 4-(((3R,5S)-4-(3-(1H-pyrrol-1-yl)benzyl)-3,5- |
| | dimethylpiperazin-1-yl)methyl)-N-hydroxybenzamide |
| 256 | 4-(((2S,6R)-4-(3-(1H-pyrrol-1-yl)benzyl)-2,6- |
| | dimethylpiperazin-1-yl)methyl)-N-hydroxybenzamide |
| 257 | 4-(((2S,6R)-4-(3-(furan-2-yl)benzoyl)-2,6- |
| | dimethylpiperazin-1-yl)methyl)-N-hydroxybenzamide |
| 258 | 4-(((2S,6R)-4-(3-(furan-3-yl)benzoyl)-2,6- |
| | dimethylpiperazin-1-yl)methyl)-N-hydroxybenzamide |
| 259 | 4-(((2S,6R)-4-(3-(3,6-dihydro-2H-pyran-4-yl)benzoyl)- |
| | 2,6-dimethylpiperazin-1-yl)methyl)-N-hydroxybenzamide |
| 260 | 4-(((2S,6R)-2,6-dimethyl-4-(3-(pyridin-4- |
| | yl)benzoyl)piperazin-1-yl)methyl)-N-hydroxybenzamide |
| 261 | 4-(((2S,6R)-2,6-dimethyl-4-(3-(pyridin-3- |
| | yl)benzoyl)piperazin-1-yl)methyl)-N-hydroxybenzamide |
| 262 | 4-(((2S,6R)-4-(3-(2-fluoropyridin-4-yl)benzoyl)-2,6- |
| | dimethylpiperazin-1-yl)methyl)-N-hydroxybenzamide |
| 263 | 4-(((2S,6R)-4-(4′,4′-dimethyl-2′,3′,4′,5′-tetrahydro- |
| | [1,1′-biphenyl]-3-carbonyl)-2,6-dimethylpiperazin-1- |
| | yl)methyl)-N-hydroxybenzamide |
| 265 | 4-(((3R,5S)-3,5-dimethyl-4-(naphthalen-2- |
| | ylmethyl)piperazin-1-yl)methyl)-N-hydroxybenzamide |
| 266 | 4-(((3R,5S)-3,5-dimethyl-4-(pyridin-4- |
| | ylmethyl)piperazin-1-yl)methyl)-N-hydroxybenzamide |
| | (Trifluoroacetic acid salt) |
| 267 | 4-(((3R,5S)-4-(3-chloro-2-(trifluoromethyl)benzyl)- |
| | 3,5-dimethylpiperazin-1-yl)methyl)-N-hydroxybenzamide |
| 268 | 4-(((2S,6R)-2,6-dimethyl-4-(naphthalen-2- |
| | ylmethyl)piperazin-1-yl)methyl)-N-hydroxybenzamide |
| 270 | N-hydroxy-4-(((3R,5S)-4-isopentyl-3,5- |
| | dimethylpiperazin-1-yl)methyl)benzamide |
| | (Trifluoroacetic acid salt) |
| 271 | N-hydroxy-4-(((2S,6R)-4-isopentyl-2,6- |
| | dimethylpiperazin-1-yl)methyl)benzamide |
| | (Trifluoroacetic acid salt) |
| 272 | 4-(((3R,5S)-3,5-dimethyl-4-(3-methylbut-2-en-1- |
| | yl)piperazin-1-yl)methyl)-N-hydroxybenzamide |
| 273 | N-hydroxy-4-(((2S,6R)-4-isopropyl-2,6- |
| | dimethylpiperazin-1-yl)methyl)benzamide |
| | (Trifluoroacetic acid salt) |
| 274 | 4-(((2S,6R)-4-butyl-2,6-dimethylpiperazin-1- |
| | yl)methyl)-N-hydroxybenzamide (Trifluoroacetic acid |
| | salt) |
| 275 | 4-(((3R,5S)-4-butyl-3,5-dimethylpiperazin-1- |
| | yl)methyl)-N-hydroxybenzamide (Trifluoroacetic acid |
| | salt) |
| 276 | 4-(((2S,6R)-4-(2-(furan-2-yl)benzoyl)-2,6- |
| | dimethylpiperazin-1-yl)methyl)-N-hydroxybenzamide |
| 277 | 4-(((2S,6R)-4-(2-(furan-3-yl)benzoyl)-2,6- |
| | dimethylpiperazin-1-yl)methyl)-N-hydroxybenzamide |
| 278 | 4-(((2S,6R)-4-(2-(3,6-dihydro-2H-pyran-4-yl)benzoyl)- |
| | 2,6-dimethylpiperazin-1-yl)methyl)-N-hydroxybenzamide |
| 279 | 4-(((2S,6R)-2,6-dimethyl-4-(2-(pyridin-3- |
| | yl)benzoyl)piperazin-1-yl)methyl)-N-hydroxybenzamide |
| 280 | 4-(((2S,6R)-4-(4′,4′-dimethyl-2′,3′,4′,5′-tetrahydro- |
| | [1,1′-biphenyl]-2-carbonyl)-2,6-dimethylpiperazin-1- |
| | yl)methyl)-N-hydroxybenzamide |
| 283 | 4-(((3R,5S)-4-(2-(furan-2-yl)benzoyl)-3,5- |
| | dimethylpiperazin-1-yl)methyl)-N-hydroxybenzamide |
| 284 | 4-(((3R,5S)-4-(2-(furan-3-yl)benzoyl)-3,5- |
| | dimethylpiperazin-1-yl)methyl)-N-hydroxybenzamide |
| 285 | 4-(((3R,5S)-3,5-dimethyl-4-(2-(pyridin-4- |
| | yl)benzoyl)piperazin-1-yl)methyl)-N-hydroxybenzamide |
| 286 | 4-(((3R,5S)-3,5-dimethyl-4-(2-(pyridin-3- |
| | yl)benzoyl)piperazin-1-yl)methyl)-N-hydroxybenzamide |
| 288 | 4-(((3R,5S)-4-(4′,4′-dimethyl-2′,3′,4′,5′-tetrahydro- |
| | [1,1′-biphenyl]-2-carbonyl)-3,5-dimethylpiperazin-1- |
| | yl)methyl)-N-hydroxybenzamide |
| 290 | 4-(((3R,5S)-3,5-dimethyl-4-(4- |
| | methylbenzoyl)piperazin-1-yl)methyl)-N- |
| | hydroxybenzamide (Trifluoroacetic acid salt) |
| 291 | N-hydroxy-4-(((3R,5S)-4-(4-methoxybenzoyl)-3,5- |
| | dimethylpiperazin-1-yl)methyl)benzamide |
| | (Trifluoroacetic acid salt) |
| 292 | 4-(((3R,5S)-3,5-dimethyl-4-pivaloylpiperazin-1- |
| | yl)methyl)-N-hydroxybenzamide (Trifluoroacetic acid |
| | salt) |
| 293 | 4-(((3R,5S)-3,5-dimethyl-4-propionylpiperazin-1- |
| | yl)methyl)-N-hydroxybenzamide (Trifluoroacetic acid |
| | salt) |
| 294 | 4-(((3R,5S)-4-butyryl-3,5-dimethylpiperazin-1- |
| | yl)methyl)-N-hydroxybenzamide (Trifluoroacetic acid |
| | salt) |
| 295 | 4-(((3R,5S)-4-(cyclopropanecarbonyl)-3,5- |
| | dimethylpiperazin-1-yl)methyl)-N-hydroxybenzamide |
| | (Trifluoroacetic acid salt) |
| 296 | N-hydroxy-4-(((3R,5S)-4-isobutyryl-3,5- |
| | dimethylpiperazin-1-yl)methyl)benzamide |
| | (Trifluoroacetic acid salt) |
| 297 | 4-(((3R,5S)-3,5-dimethyl-4-(3- |
| | methylbutanoyl)piperazin-1-yl)methyl)-N- |
| | hydroxybenzamide (Trifluoroacetic acid salt) |
| 298 | 4-(((2S,6R)-2,6-dimethyl-4-(3- |
| | (trifluoromethyl)benzyl)piperazin-1-yl)methyl)-N- |
| | hydroxybenzamide |
| 299 | 4-(((2S,6R)-4-(4-(dimethylamino)benzyl)-2,6- |
| | dimethylpiperazin-1-yl)methyl)-N-hydroxybenzamide |
| 300 | 4-(((2S,6R)-2,6-dimethyl-4-(4- |
| | (methylsulfonyl)benzyl)piperazin-1-yl)methyl)-N- |
| | hydroxybenzamide |
| 301 | 4-(((2S,6R)-4-(4-(1H-imidazol-1-yl)benzyl)-2,6- |
| | dimethylpiperazin-1-yl)methyl)-N-hydroxybenzamide |
| 302 | 4-(((2S,6R)-2,6-dimethyl-4-(4-(thiophen-2- |
| | yl)benzyl)piperazin-1-yl)methyl)-N-hydroxybenzamide |
| 303 | 4-(((2S,6R)-4-(4-(furan-2-yl)benzyl)-2,6- |
| | dimethylpiperazin-1-yl)methyl)-N-hydroxybenzamide |
| 304 | 4-(((2S,6R)-4-(4-(4H-1,2,4-triazol-4-yl)benzyl)-2,6- |
| | dimethylpiperazin-1-yl)methyl)-N-hydroxybenzamide |
| 305 | 4-(((2S,6R)-2,6-dimethyl-4-(2-(2-methyl-1H-imidazol- |
| | 1-yl)benzyl)piperazin-1-yl)methyl)-N-hydroxybenzamide |
| 309 | 4-(((3R,5S)-4-(4-(furan-3-yl)benzyl)-3,5- |
| | dimethylpiperazin-1-yl)methyl)-N-hydroxybenzamide |
| 321 | (2S,6R)-N-(3-(1H-pyrrol-1-yl)phenyl)-4-(4-((E)-3- |
| | (hydroxyamino)-3-oxoprop-1-en-1-yl)benzyl)-2,6- |
| | dimethylpiperazine-1-carboxamide |
| 322 | (E)-3-(4-(((3R,5S)-4-(furan-2-carbonyl)-3,5- |
| | dimethylpiperazin-1-yl)methyl)phenyl)-N- |
| | hydroxyacrylamide |
| 323 | (E)-3-(4-(((3R,5S)-3,5-dimethyl-4-(2- |
| | phenylacetyl)piperazin-1-yl)methyl)phenyl)-N- |
| | hydroxyacrylamide |
| 326 | 4-(((3R,5S)-4-(2-(3,6-dihydro-2H-pyran-4-yl)benzoyl)- |
| | 3,5-dimethylpiperazin-1-yl)methyl)-N-hydroxybenzamide |
| 327 | 4-(((3R,5S)-4-(2-(furan-2-yl)benzyl)-3,5- |
| | dimethylpiperazin-1-yl)methyl)-N-hydroxybenzamide |
| 328 | 4-(((3R,5S)-4-(2-(furan-3-yl)benzyl)-3,5- |
| | dimethylpiperazin-1-yl)methyl)-N-hydroxybenzamide |
| 329 | 4-(((3R,5S)-3,5-dimethyl-4-(2-(pyridin-4- |
| | yl)benzyl)piperazin-1-yl)methyl)-N-hydroxybenzamide |
| 330 | 4-(((3R,5S)-3,5-dimethyl-4-(2-(pyridin-3- |
| | yl)benzyl)piperazin-1-yl)methyl)-N-hydroxybenzamide |
| 331 | 4-(((3R,5S)-4-((4′,4′-dimethyl-2′,3′,4′,5′- |
| | tetrahydro-[1,1′-biphenyl]-2-yl)methyl)-3,5- |
| | dimethylpiperazin-1-yl)methyl)-N-hydroxybenzamide |
| 332 | 4-(((3R,5S)-4-(2-(3,6-dihydro-2H-pyran-4-yl)benzyl)- |
| | 3,5-dimethylpiperazin-1-yl)methyl)-N-hydroxybenzamide |
| 340 | 4-(((3R,5S)-4-(4-(furan-2-yl)benzoyl)-3,5- |
| | dimethylpiperazin-1-yl)methyl)-N-hydroxybenzamide |
| 342 | 4-(((3R,5S)-4-(3-(furan-2-yl)benzyl)-3,5- |
| | dimethylpiperazin-1-yl)methyl)-N-hydroxybenzamide |
| 343 | 4-(((3R,5S)-4-(3-(furan-3-yl)benzyl)-3,5- |
| | dimethylpiperazin-1-yl)methyl)-N-hydroxybenzamide |
| 344 | 4-(((3R,5S)-3,5-dimethyl-4-(3-(pyridin-3- |
| | yl)benzyl)piperazin-1-yl)methyl)-N-hydroxybenzamide |
| 345 | 4-(((3R,5S)-3,5-dimethyl-4-(3-(pyridin-4- |
| | yl)benzyl)piperazin-1-yl)methyl)-N-hydroxybenzamide |
| 346 | 4-(((3R,5S)-4-(3-(3,6-dihydro-2H-pyran-4-yl)benzyl)- |
| | 3,5-dimethylpiperazin-1-yl)methyl)-N-hydroxybenzamide |
| 347 | 4-(((3R,5S)-4-((4′,4′-dimethyl-2′,3′,4′,5′- |
| | tetrahydro-[1,1′-biphenyl]-3-yl)methyl)-3,5- |
| | dimethylpiperazin-1-yl)methyl)-N-hydroxybenzamide |
| 348 | 4-(((2S,6R)-4-(2-(furan-3-yl)benzyl)-2,6- |
| | dimethylpiperazin-1-yl)methyl)-N-hydroxybenzamide |
| 349 | 4-(((2S,6R)-2,6-dimethyl-4-(2-(pyridin-3- |
| | yl)benzyl)piperazin-1-yl)methyl)-N-hydroxybenzamide |
| 350 | 4-(((2S,6R)-2,6-dimethyl-4-(2-(pyridin-4- |
| | yl)benzyl)piperazin-1-yl)methyl)-N-hydroxybenzamide |
| 351 | 4-(((2S,6R)-4-(2-(3,6-dihydro-2H-pyran-4-yl)benzyl)- |
| | 2,6-dimethylpiperazin-1-yl)methyl)-N-hydroxybenzamide |
| 352 | 4-(((2S,6R)-4-((4′,4′-dimethyl-2′,3′,4′,5′- |
| | tetrahydro-[1,1′-biphenyl]-2-yl)methyl)-2,6- |
| | dimethylpiperazin-1-yl)methyl)-N-hydroxybenzamide |
| 354 | 4-(((3R,5S)-3,5-dimethyl-4-(oxazol-4- |
| | ylmethyl)piperazin-1-yl)methyl)-N-hydroxybenzamide |
| 355 | 4-(((3R,5S)-3,5-dimethyl-4-(pyrimidin-5- |
| | ylmethyl)piperazin-1-yl)methyl)-N-hydroxybenzamide |
| 356 | 4-(((3R,5S)-3,5-dimethyl-4-(3- |
| | (morpholinomethyl)benzyl)piperazin-1-yl)methyl)-N- |
| | hydroxybenzamide |
| 376 | 4-(((3R,5S)-3,5-dimethyl-4-(4- |
| | (morpholinomethyl)benzyl)piperazin-1-yl)methyl)-N- |
| | hydroxybenzamide |
| 380 | 4-(((3R,5S)-4-(3-((1-(2-fluoro-2- |
| | methylpropyl)piperidin-4-yl)methoxy)benzyl)-3,5- |
| | dimethylpiperazin-1-yl)methyl)-N-hydroxybenzamide |
| 382 | 4-(((3R,5S)-3,5-dimethyl-4-(3-(piperidin-1- |
| | ylmethyl)benzyl)piperazin-1-yl)methyl)-N- |
| | hydroxybenzamide |
| 383 | 4-(((3R,5S)-4-(3-(((R)-3-fluoropyrrolidin-1- |
| | yl)methyl)benzyl)-3,5-dimethylpiperazin-1-yl)methyl)- |
| | N-hydroxybenzamide |
| 384 | 4-(((3R,5S)-4-(3-((3,3-difluoroazetidin-1- |
| | yl)methyl)benzyl)-3,5-dimethylpiperazin-1-yl)methyl)- |
| | N-hydroxybenzamide |
| 385 | 4-(((3R,5S)-4-(3-((4-benzylpiperazin-1- |
| | yl)methyl)benzyl)-3,5-dimethylpiperazin-1-yl)methyl)- |
| | N-hydroxybenzamide |
| 386 | N-hydroxy-4-(((3R,5S)-4-(3-((4-(2-hydroxy-2- |
| | methylpropyl)piperazin-1-yl)methyl)benzyl)-3,5- |
| | dimethylpiperazin-1-yl)methyl)benzamide |
| 387 | 4-(((3R,5S)-4-(3-((4-(2-fluoro-2- |
| | methylpropyl)piperazin-1-yl)methyl)benzyl)-3,5- |
| | dimethylpiperazin-1-yl)methyl)-N-hydroxybenzamide |
| 388 | 4-(((3R,5S)-4-(4-((1-(2-fluoro-2- |
| | methylpropyl)piperidin-4-yl)methoxy)benzyl)-3,5- |
| | dimethylpiperazin-1-yl)methyl)-N-hydroxybenzamide |
| 396 | 4-(((2S,6R)-4-(3-(furan-3-yl)benzyl)-2,6- |
| | dimethylpiperazin-1-yl)methyl)-N-hydroxybenzamide |
| 400 | 4-(((2S,6R)-4-((4′,4′-dimethyl-2′,3′,4′,5′- |
| | tetrahydro-[1,1′-biphenyl]-4-yl)methyl)-2,6- |
| | dimethylpiperazin-1-yl)methyl)-N-hydroxybenzamide |
| 401 | 4-(((2S,6R)-4-(4-(3,6-dihydro-2H-pyran-4-yl)benzyl)- |
| | 2,6-dimethylpiperazin-1-yl)methyl)-N-hydroxybenzamide |
| 402 | 4-(((3R,5S)-3,5-dimethyl-4-(tetrahydrofuran-2- |
| | carbonyl)piperazin-1-yl)methyl)-N-hydroxybenzamide |
| 403 | 4-(((3R,5S)-3,5-dimethyl-4-(1H-pyrazole-3- |
| | carbonyl)piperazin-1-yl)methyl)-N-hydroxybenzamide |
| 404 | 4-(((3R,5S)-3,5-dimethyl-4-(pyrazine-2- |
| | carbonyl)piperazin-1-yl)methyl)-N-hydroxybenzamide |
| 405 | N-hydroxy-4-(((3R,5S)-4-isonicotinoyl-3,5- |
| | dimethylpiperazin-1-yl)methyl)benzamide |
| 411 | 4-(((3R,5S)-4-(4-(((R)-3-fluoropyrrolidin-1- |
| | yl)methyl)benzyl)-3,5-dimethylpiperazin-1-yl)methyl)- |
| | N-hydroxybenzamide |
| 412 | 4-(((3R,5S)-4-(4-((3,3-difluoroazetidin-1- |
| | yl)methyl)benzyl)-3,5-dimethylpiperazin-1-yl)methyl)- |
| | N-hydroxybenzamide |
| 413 | 4-(((3R,5S)-4-(4-((4-benzylpiperazin-1- |
| | yl)methyl)benzyl)-3,5-dimethylpiperazin-1-yl)methyl)- |
| | N-hydroxybenzamide |
| 423 | 4-(((3R,5S)-3,5-dimethyl-4-(3-(pyrrolidin-1- |
| | ylmethyl)benzoyl)piperazin-1-yl)methyl)-N- |
| | hydroxybenzamide |
| 424 | 4-(((3R,5S)-3,5-dimethyl-4-(3-(piperidin-1- |
| | ylmethyl)benzoyl)piperazin-1-yl)methyl)-N- |
| | hydroxybenzamide |
| 425 | 4-(((3R,5S)-4-(3-((diethylamino)methyl)benzoyl)-3,5- |
| | dimethylpiperazin-1-yl)methyl)-N-hydroxybenzamide |
| 426 | 4-(((3R,5S)-4-(3-((diethylamino)methyl)benzyl)-3,5- |
| | dimethylpiperazin-1-yl)methyl)-N-hydroxybenzamide |
| 427 | 4-(((3R,5S)-3,5-dimethyl-4-(3-(pyrrolidin-1- |
| | ylmethyl)benzyl)piperazin-1-yl)methyl)-N- |
| | hydroxybenzamide |
| 428 | 4-(((3R,5S)-3,5-dimethyl-4-(3-((4-methylpiperazin-1- |
| | yl)methyl)benzyl)piperazin-1-yl)methyl)-N- |
| | hydroxybenzamide |
| 429 | 4-(((3R,5S)-4-(3-((4-ethylpiperazin-1- |
| | yl)methyl)benzyl)-3,5-dimethylpiperazin-1-yl)methyl)- |
| | N-hydroxybenzamide |
| 430 | N-hydroxy-4-(((3R,5S)-4-(3-((4-isopropylpiperazin-1- |
| | yl)methyl)benzyl)-3,5-dimethylpiperazin-1- |
| | yl)methyl)benzamide |
| 431 | 4-(((3R,5S)-4-(3-((4-acetylpiperazin-1- |
| | yl)methyl)benzyl)-3,5-dimethylpiperazin-1-yl)methyl)- |
| | N-hydroxybenzamide |
| 432 | 4-(((3R,5S)-3,5-dimethyl-4-(3-((4-methylpiperazin-1- |
| | yl)methyl)benzoyl)piperazin-1-yl)methyl)-N- |
| | hydroxybenzamide |
| 433 | 4-(((3R,5S)-4-(3-((4-ethylpiperazin-1- |
| | yl)methyl)benzoyl)-3,5-dimethylpiperazin-1- |
| | yl)methyl)-N-hydroxybenzamide |
| 434 | N-hydroxy-4-(((3R,5S)-4-(3-((4-isopropylpiperazin-1- |
| | yl)methyl)benzoyl)-3,5-dimethylpiperazin-1- |
| | yl)methyl)benzamide |
| 435 | 4-(((3R,5S)-4-(3-((4-acetylpiperazin-1- |
| | yl)methyl)benzoyl)-3,5-dimethylpiperazin-1- |
| | yl)methyl)-N-hydroxybenzamide |
| 439 | 4-(((3R,5S)-3,5-dimethyl-4-(3- |
| | (morpholinomethyl)benzoyl)piperazin-1-yl)methyl)-N- |
| | hydroxybenzamide |
| 440 | 4-(((3R,5S)-4-(3-(((1-(diethylamino)propan-2- |
| | yl)amino)methyl)benzoyl)-3,5-dimethylpiperazin-1- |
| | yl)methyl)-N-hydroxybenzamide |
| 441 | 4-(((3R,5S)-4-(3-(((1-(benzyl(methyl)amino)propan-2- |
| | yl)amino)methyl)benzoyl)-3,5-dimethylpiperazin-1- |
| | yl)methyl)-N-hydroxybenzamide |
| 442 | N-hydroxy-4-(((3R,5S)-4-(3-((4-isopentylpiperazin-1- |
| | yl)methyl)benzoyl)-3,5-dimethylpiperazin-1- |
| | yl)methyl)benzamide |
| 443 | 4-(((3R,5S)-3,5-dimethyl-4-(3-((4-(2,2,2- |
| | trifluoroethyl)piperazin-1- |
| | yl)methyl)benzoyl)piperazin-1-yl)methyl)-N- |
| | hydroxybenzamide |
| 444 | 4-(((3R,5S)-3,5-dimethyl-4-(3-((4- |
| | (methylsulfonyl)piperazin-1- |
| | yl)methyl)benzoyl)piperazin-1-yl)methyl)-N- |
| | hydroxybenzamide |
| 446 | 4-(((3R,5S)-3,5-dimethyl-4-(3-(morpholine-4- |
| | carbonyl)benzyl)piperazin-1-yl)methyl)-N- |
| | hydroxybenzamide |
| 452 | 4-(((3R,5S)-3,5-dimethyl-4-(3-((4- |
| | (methylsulfonyl)piperazin-1- |
| | yl)methyl)benzyl)piperazin-1-yl)methyl)-N- |
| | hydroxybenzamide |
| 453 | 4-(((3R,5S)-3,5-dimethyl-4-(3-((4-(2,2,2- |
| | trifluoroethyl)piperazin-1- |
| | yl)methyl)benzyl)piperazin-1-yl)methyl)-N- |
| | hydroxybenzamide |
| 454 | N-hydroxy-4-(((3R,5S)-4-(3-((4-isopentylpiperazin-1- |
| | yl)methyl)benzyl)-3,5-dimethylpiperazin-1- |
| | yl)methyl)benzamide |
| 455 | 4-(((3R,5S)-4-(3-(((1-(diethylamino)propan-2- |
| | yl)amino)methyl)benzyl)-3,5-dimethylpiperazin-1- |
| | yl)methyl)-N-hydroxybenzamide |
| 456 | 4-(((3R,5S)-4-(3-(((1-(benzyl(methyl)amino)propan-2- |
| | yl)amino)methyl)benzyl)-3,5-dimethylpiperazin-1- |
| | yl)methyl)-N-hydroxybenzamide |
| 457 | 4-(((3R,5S)-3,5-dimethyl-4-(2- |
| | (morpholinomethyl)benzyl)piperazin-1-yl)methyl)-N- |
| | hydroxybenzamide |
| 458 | 4-(((3R,5S)-3,5-dimethyl-4-(4- |
| | (morpholinomethyl)benzoyl)piperazin-1-yl)methyl)-N- |
| | hydroxybenzamide |
| 459 | 4-(((3R,5S)-3,5-dimethyl-4-(4-(piperidin-1- |
| | ylmethyl)benzoyl)piperazin-1-yl)methyl)-N- |
| | hydroxybenzamide |
| 460 | 4-(((3R,5S)-4-(4-(((R)-3-fluoropyrrolidin-1- |
| | yl)methyl)benzoyl)-3,5-dimethylpiperazin-1- |
| | yl)methyl)-N-hydroxybenzamide |
| 461 | 4-(((3R,5S)-3,5-dimethyl-4-(4-(pyrrolidin-1- |
| | ylmethyl)benzoyl)piperazin-1-yl)methyl)-N- |
| | hydroxybenzamide |
| 462 | 4-(((3R,5S)-3,5-dimethyl-4-(4-((4-methylpiperazin-1- |
| | yl)methyl)benzoyl)piperazin-1-yl)methyl)-N- |
| | hydroxybenzamide |
| 463 | 4-(((3R,5S)-3,5-dimethyl-4-(4-((4- |
| | (methylsulfonyl)piperazin-1- |
| | yl)methyl)benzoyl)piperazin-1-yl)methyl)-N- |
| | hydroxybenzamide |
| 466 | 4-(((3R,5S)-3,5-dimethyl-4-(4-(pyrrolidin-1- |
| | ylmethyl)benzyl)piperazin-1-yl)methyl)-N- |
| | hydroxybenzamide |
| 467 | 4-(((3R,5S)-3,5-dimethyl-4-(4-(piperidin-1- |
| | ylmethyl)benzyl)piperazin-1-yl)methyl)-N- |
| | hydroxybenzamide |
| 468 | 4-(((3R,5S)-3,5-dimethyl-4-(4-((4-methylpiperazin-1- |
| | yl)methyl)benzyl)piperazin-1-yl)methyl)-N- |
| | hydroxybenzamide |
| 472 | (E)-3-(4-(((3R,5S)-3,5-dimethyl-4-(3- |
| | (morpholinomethyl)benzyl)piperazin-1- |
| | yl)methyl)phenyl)-N-hydroxyacrylamide |
| 475 | (E)-3-(4-(((2S,6R)-2,6-dimethyl-4-(3-(pyrrolidin-1- |
| | ylmethyl)benzyl)piperazin-1-yl)methyl)phenyl)-N- |
| | hydroxyacrylamide |
| 476 | (E)-3-(4-(((2S,6R)-4-(3- |
| | ((diethylamino)methyl)benzyl)-2,6-dimethylpiperazin- |
| | 1-yl)methyl)phenyl)-N-hydroxyacrylamide |
| 477 | (E)-3-(4-(((2S,6R)-4-(4-(furan-2-yl)benzyl)-2,6- |
| | dimethylpiperazin-1-yl)methyl)phenyl)-N- |
| | hydroxyacrylamide |
| 478 | 4-(((2S,6R)-4-(3-((diethylamino)methyl)benzyl)-2,6- |
| | dimethylpiperazin-1-yl)methyl)-N-hydroxybenzamide |
| | (hydrochloride salt) |
| 479 | 4-(((2S,6R)-2,6-dimethyl-4-(3-(pyrrolidin-1- |
| | ylmethyl)benzyl)piperazin-1-yl)methyl)-N- |
| | hydroxybenzamide |
| 480 | (E)-3-(4-(((2S,6R)-2,6-dimethyl-4-(3-(pyrrolidin-1- |
| | ylmethyl)benzoyl)piperazin-1-yl)methyl)phenyl)-N- |
| | hydroxyacrylamide |
| 481 | 4-(((3R,5S)-3,5-dimethyl-4-(2-(1-(methylsulfonyl)- |
| | 1,2,3,6-tetrahydropyridin-4-yl)benzyl)piperazin-1- |
| | yl)methyl)-N-hydroxybenzamide |
| 482 | 4-(((3R,5S)-4-(2-(1-acetyl-1,2,3,6-tetrahydropyridin- |
| | 4-yl)benzyl)-3,5-dimethylpiperazin-1-yl)methyl)-N- |
| | hydroxybenzamide |
| 483 | 4-(((3R,5S)-3,5-dimethyl-4-(2-(1-(2,2,2- |
| | trifluoroethyl)-1,2,3,6-tetrahydropyridin-4- |
| | yl)benzyl)piperazin-1-yl)methyl)-N-hydroxybenzamide |
| 484 | N-hydroxy-4-(((3R,5S)-4-(2-(1-isopropyl-1,2,3,6- |
| | tetrahydropyridin-4-yl)benzyl)-3,5-dimethylpiperazin- |
| | 1-yl)methyl)benzamide |
| 485 | 4-(((3R,5S)-4-(2-(1-ethyl-1,2,3,6-tetrahydropyridin- |
| | 4-yl)benzyl)-3,5-dimethylpiperazin-1-yl)methyl)-N- |
| | hydroxybenzamide |
| 486 | 4-(((3R,5S)-4-(4-((4-acetylpiperazin-1- |
| | yl)methyl)benzyl)-3,5-dimethylpiperazin-1-yl)methyl)- |
| | N-hydroxybenzamide (hydrochloride salt) |
| 487 | (E)-3-(4-(((2S,6R)-4-(3- |
| | ((diethylamino)methyl)benzoyl)-2,6-dimethylpiperazin- |
| | 1-yl)methyl)phenyl)-N-hydroxyacrylamide |
| 520 | 3-(((3R,5S)-4-benzyl-3,5-dimethylpiperazin-1- |
| | yl)methyl)-N-hydroxy-1H-indole-6-carboxamide |
| 569 | 5-(((3R,5S)-4-benzyl-3,5-dimethylpiperazin-1- |
| | yl)methyl)-N-hydroxy-1H-indole-2-carboxamide |
| 571 | 5-(((3R,5S)-4-benzyl-3,5-dimethylpiperazin-1- |
| | yl)methyl)-N-hydroxybenzofuran-2-carboxamide |
| 573 | 6-(((3R,5S)-4-benzyl-3,5-dimethylpiperazin-1- |
| | yl)methyl)-N-hydroxy-1H-indole-2-carboxamide |
| 574 | 2-(((3R,5S)-4-benzyl-3,5-dimethylpiperazin-1- |
| | yl)methyl)-N-hydroxybenzofuran-5-carboxamide |
| 609 | 5-(((3R,5S)-3,5-dimethyl-4-(3- |
| | (morpholinomethyl)benzyl)piperazin-1-yl)methyl)-N- |
| | hydroxypicolinamide |
| 652 | 4-(((3R,5S)-4-benzyl-3,5-dimethylpiperazin-1- |
| | yl)methyl)-N,2-dihydroxybenzamide |
| 653 | 4-(((3R,5S)-3,5-dimethyl-4-(3- |
| | (morpholinomethyl)benzyl)piperazin-1-yl)methyl)-N,2- |
| | dihydroxybenzamide |
| 696 | 4-(((3R,5S)-3,5-dimethyl-4-(3- |
| | (morpholinomethyl)benzyl)piperazin-1-yl)methyl)-3- |
| | fluoro-N-hydroxybenzamide |
| 812 | 5-(((3R,5S)-4-benzyl-3,5-dimethylpiperazin-1- |
| | yl)methyl)-N-hydroxypicolinamide |
| 813 | 4-(((3R,5S)-4-benzyl-3,5-dimethylpiperazin-1- |
| | yl)methyl)-3-fluoro-N-hydroxybenzamide |
| 814 | 4-((3R,5S)-4-benzyl-3,5-dimethylpiperazine-1- |
| | carbonyl)-N-hydroxybenzamide |
| 818 | 4-(((3R,5S)-4-benzyl-3,5-dimethylpiperazin-1- |
| | yl)sulfonyl)-N-hydroxybenzamide |
| 820 | 2-(4-(((3R,5S)-4-benzyl-3,5-dimethylpiperazin-1- |
| | yl)sulfonyl)phenyl)-N-hydroxyacetamide |
| 822 | 4-(((3R,5S)-4-benzyl-3,5-dimethylpiperazin-1- |
| | yl)methyl)-N-hydroxycyclohexane-1-carboxamide |
| 823 | 2-(4-((3R,5S)-4-benzyl-3,5-dimethylpiperazine-1- |
| | carbonyl)phenyl)-N-hydroxyacetamide |
| 824 | 3-(4-((3R,5S)-4-benzyl-3,5-dimethylpiperazine-1- |
| | carbonyl)phenyl)-N-hydroxypropanamide |

6. The pharmaceutical composition according to claim 5, wherein the compound represented by Formula I is a compound described in the following table:

| No. | Name of Compound |
|---|---|
| 081 | 4-(((2S,6R)-4-benzyl-2,6-dimethylpiperazin-1- |
| | yl)methyl)-N-hydroxybenzamide |
| 082 | 4-(((3R,5S)-4-acetyl-3,5-dimethylpiperazin-1- |
| | yl)methyl)-N-hydroxybenzamide |
| 083 | 4-(((3R,5S)-4-benzoyl-3,5-dimethylpiperazin-1- |
| | yl)methyl)-N-hydroxybenzamide |
| 084 | 4-(((3R,5S)-4-benzyl-3,5-dimethylpiperazin-1- |
| | yl)methyl)-N-hydroxybenzamide |
| 098 | (2S,6R)-4-(4-(hydroxycarbamoyl)benzyl)-N- |
| | isopropyl-2,6-dimethylpiperazine-1-carboxamide |
| 099 | (2S,6R)-N-(3-chlorophenyl)-4-(4-(hydroxycarbamoyl)benzyl)- |
| | 2,6-dimethylpiperazine-1-carboxamide |
| 100 | 4-(((3R,5S)-3,5-dimethyl-4-(phenylsulfonyl)piperazin-1-yl)methyl)- |
| | N-hydroxybenzamide |
| 106 | 4-(((2S,6R)-4-(4-chlorobenzoyl)-2,6-dimethylpiperazin-1-yl)methyl)- |
| | N-hydroxybenzamide |
| 107 | 4-(((2S,6R)-4-(3-chlorobenzoyl)-2,6-dimethylpiperazin- |
| | 1-yl)methyl)-N-hydroxybenzamide |
| 108 | 4-(((2S,6R)-4-(2-chlorobenzoyl)-2,6-dimethylpiperazin- |
| | 1-yl)methyl)-N-hydroxybenzamide |
| 109 | 4-(((2S,6R)-4-((4-chlorophenyl)sulfonyl)-2,6-dimethylpiperazin- |
| | 1-yl)methyl)-N-hydroxybenzamide |
| 110 | 4-(((2S,6R)-4-((2-chlorophenyl)sulfonyl)-2,6-dimethylpiperazin- |
| | 1-yl)methyl)-N-hydroxybenzamide |
| 112 | 4-(((2S,6R)-2,6-dimethyl-4-nicotinoylpiperazin-1- |
| | yl)methyl)-N-hydroxybenzamide |
| 120 | 4-(((3R,5S)-4-(2-chlorobenzoyl)-3,5-dimethylpiperazin-1- |
| | yl)methyl)-N-hydroxybenzamide |
| 121 | 4-(((3R,5S)-4-(3-chlorobenzoyl)-3,5-dimethylpiperazin-1- |
| | yl)methyl)-N-hydroxybenzamide |
| 122 | 4-(((3R,5S)-4-(4-chlorobenzoyl)-3,5-dimethylpiperazin-1- |
| | yl)methyl)-N-hydroxybenzamide |
| 123 | 4-(((3R,5S)-3,5-dimethyl-4-picolinoylpiperazin-1-yl)methyl)-N- |
| | hydroxybenzamide |
| 125 | 4-(((3R,5S)-4-(furan-2-carbonyl)-3,5-dimethylpiperazin-1-yl)methyl)- |
| | N-hydroxybenzamide |
| 126 | 4-(((3R,5S)-3,5-dimethyl-4-(thiophene-2-carbonyl)piperazin-1- |
| | yl)methyl)-N-hydroxybenzamide |
| 127 | 4-(((3R,5S)-4-(2-chlorobenzyl)-3,5-dimethylpiperazin-1- |
| | yl)methyl)-N-hydroxybenzamide |
| 128 | 4-(((3R,5S)-4-(3-chlorobenzyl)-3,5-dimethylpiperazin-1- |
| | yl)methyl)-N-hydroxybenzamide |
| 145 | 4-(((3R,5S)-4-(furan-2-yl)methyl)-3,5-dimethylpiperazin- |
| | 1-yl)methyl)-N-hydroxybenzamide |
| 146 | 4-(((3R,5S)-3,5-dimethyl-4-(2-phenylacetyl)piperazin-1- |
| | yl)methyl)-N-hydroxybenzamide |
| 147 | 4-(((3R,5S)-4-ethyl-3,5-dimethylpiperazin-1-yl)methyl)-N- |
| | hydroxybenzamide |
| 148 | 4-(((3R,5S)-3,5-dimethyl-4-propylpiperazin-1-yl)methyl)- |
| | N-hydroxybenzamide |
| 149 | 4-(((3R,5S)-3,5-dimethyl-4-(2,2,2-trifluoroethyl)piperazin- |
| | 1-yl)methyl)-N-hydroxybenzamide |
| 159 | (2S,6R)-4-(4-(hydroxycarbamoyl)benzyl)-2,6-dimethyl-N- |
| | phenylpiperazine-1-carboxamide |
| 160 | (2S,6R)-4-(4-(hydroxycarbamoyl)benzyl)-2,6-dimethyl-N-(o-tolyl)piperazine- |
| | 1-carboxamide |
| 161 | (2S,6R)-N-benzyl-4-(4-(hydroxycarbamoyl)benzyl)-2,6- |
| | dimethylpiperazine-1-carboxamide |
| 171 | 4-(((3R,5S)-4-benzyl-3,5-dimethylpiperazin-1-yl)methyl)- |
| | 2-fluoro-N-hydroxybenzamide |
| 173 | 2-(4-(((3R,5S)-4-benzyl-3,5-dimethylpiperazin-1-yl)methyl)phenyl)- |
| | N-hydroxyacetamide |
| 174 | 4-(((2S,6R)-4-benzyl-2,6-dimethylpiperazin-1-yl)methyl)- |
| | 2-fluoro-N-hydroxybenzamide |
| 175 | (E)-3-(4-(((2S,6R)-4-benzyl-2,6-dimethylpiperazin-1- |
| | yl)methyl)phenyl)-N-hydroxyacrylamide |
| 177 | phenyl (2S,6R)-4-(4-(hydroxycarbamoyl)benzyl)-2,6- |
| | dimethylpiperazine-1-carboxylate |
| 186 | (3R,5S)-N-(3-(1H-pyrrol-1-yl)phenyl)-4-(4-(hydroxycarbamoyl)benzyl)- |
| | 3,5-dimethylpiperazine-1-carboxamide |
| 188 | 4-(((3R,5S)-3,5-dimethyl-4-(3-phenylpropanoyl)piperazin- |
| | 1-yl)methyl)-N-hydroxybenzamide |
| 193 | 4-(((2S,6R)-4-(2-fluoro-2-methylpropyl)-2,6-dimethylpiperazin-1- |
| | yl)methyl)-N-hydroxybenzamide |
| 194 | 6-(((3R,5S)-4-benzyl-3,5-dimethylpiperazin-1-yl)methyl)- |
| | N-hydroxynicotinamide |
| 195 | 6-(((2S,6R)-4-benzyl-2,6-dimethylpiperazin-1-yl)methyl)- |
| | N-hydroxynicotinamide |
| 196 | (3R,5S)-4-(4-(hydroxycarbamoyl)benzyl)-N,3,5-trimethyl- |
| | N-phenylpiperazine-1-carboxamide |
| 198 | (3R,5S)-N-butyl-4-(4-(hydroxycarbamoyl)benzyl)-3,5- |
| | dimethyl-N-phenylpiperazine-1-carboxamide |
| 211 | 4-(((3R,5S)-4-(2-fluorobenzyl)-3,5-dimethylpiperazin-1- |
| | yl)methyl)-N-hydroxybenzamide |
| 214 | 4-(((3R,5S)-4-(3,5-bis(trifluoromethyl)benzyl)-3,5- |
| | dimethylpiperazin-1-yl)methyl)-N-hydroxybenzamide |
| 219 | 4-(1-((3R,5S)-4-benzyl-3,5-dimethylpiperazin-1- |
| | yl)ethyl)-N-hydroxybenzamide |
| 248 | 4-(((3R,5R)-4-benzyl-3,5-dimethylpiperazin-1-yl)methyl)- |
| | N-hydroxybenzamide |
| 249 | 4-(((3R,5R)-4-(furan-2-carbonyl)-3,5-dimethylpiperazin- |
| | 1-yl)methyl)-N-hydroxybenzamide |
| 250 | 4-(((2S,6R)-4-(2-([1,1′-biphenyl]-3-yl)acetyl)-2,6- |
| | dimethylpiperazin-1-yl)methyl)-N-hydroxybenzamide |
| | (Trifluoroacetic acid salt) |
| 251 | 4-(((2S,6R)-2,6-dimethyl-4-(2-(3-(pyridin-4- |
| | yl)phenyl)acetyl)piperazin- |
| | 1-yl)methyl)-N-hydroxybenzamide |
| | (Trifluoroacetic acid salt) |
| 252 | 4-(((2S,6R)-4-(2-(3-(3,6-dihydro-2H-pyran-4-yl)phenyl)acetyl)-2,6- |
| | dimethylpiperazin-1-yl)methyl)-N-hydroxybenzamide |
| 255 | 4-(((3R,5S)-4-(3-(1H-pyrrol-1-yl)benzyl)-3,5-dimethylpiperazin- |
| | 1-yl)methyl)-N-hydroxybenzamide |
| 265 | 4-(((3R,5S)-3,5-dimethyl-4-(naphthalen-2-yl)methyl)piperazin-1- |
| | yl)methyl)-N-hydroxybenzamide |
| 266 | 4-(((3R,5S)-3,5-dimethyl-4-(pyridin-4-yl)methyl)piperazin-1-yl)methyl)-N- |
| | hydroxybenzamide (Trifluoroacetic acid salt) |
| 267 | 4-(((3R,5S)-4-(3-chloro-2-(trifluoromethyl)benzyl)-3,5- |
| | dimethylpiperazin-1-yl)methyl)-N-hydroxybenzamide |
| 272 | 4-(((3R,5S)-3,5-dimethyl-4-(3-methylbut-2-en-1- |
| | yl)piperazin-1-ylmethyl)-N-hydroxybenzamide |
| 283 | 4-(((3R,5S)-4-(2-(furan-2-yl)benzoyl)-3,5-dimethylpiperazin- |
| | 1-yl)methyl)-N-hydroxybenzamide |
| 284 | 4-(((3R,5S)-4-(2-(furan-3-yl)benzoyl)-3,5-dimethylpiperazin- |
| | 1-yl)methyl)-N-hydroxybenzamide |
| 285 | 4-(((3R,5S)-3,5-dimethyl-4-(2-(pyridin-4-yl)benzoyl)piperazin- |
| | 1-yl)methyl)-N-hydroxybenzamide |
| 286 | 4-(((3R,5S)-3,5-dimethyl-4-(2-(pyridin-3-yl)benzoyl)piperazin- |
| | 1-yl)methyl)-N-hydroxybenzamide |
| 292 | 4-(((3R,5S)-3,5-dimethyl-4-pivaloylpiperazin-1-yl)methyl)- |
| | N-hydroxybenzamide (Trifluoroacetic acid salt) |
| 295 | 4-(((3R,5S)-4-(cyclopropanecarbonyl)-3,5-dimethylpiperazin-1-yl)methyl)-N- |
| | hydroxybenzamide (Trifluoroacetic acid salt) |
| 297 | 4-(((3R,5S)-3,5-dimethyl-4-(3-methylbutanoyl)piperazin-1-yl)methyl)-N- |
| | hydroxybenzamide (Trifluoroacetic acid salt) |
| 305 | 4-(((2S,6R)-2,6-dimethyl-4-(2-(2-methyl-1H-imidazol-1-yl)benzyl)piperazin- |
| | 1-yl)methyl)-N-hydroxybenzamide |
| 326 | 4-(((3R,5S)-4-(2-(3,6-dihydro-2H-pyran-4-yl)benzoyl)-3,5-dimethylpiperazin-1- |
| | yl)methyl)-N-hydroxybenzamide |
| 328 | 4-(((3R,5S)-4-(2-(furan-3-yl)benzyl)-3,5-dimethylpiperazin- |
| | 1-yl)methyl)-N-hydroxybenzamide |
| 329 | 4-(((3R,5S)-3,5-dimethyl-4-(2-(pyridin-4-yl)benzyl)piperazin-1- |
| | yl)methyl)-N-hydroxybenzamide |
| 330 | 4-(((3R,5S)-3,5-dimethyl-4-(2-(pyridin-3-yl)benzyl)piperazin-1- |
| | yl)methyl)-N-hydroxybenzamide |
| 332 | 4-(((3R,5S)-4-(2-(3,6-dihydro-2H-pyran-4-yl)benzyl)-3,5-dimethylpiperazin-1- |
| | yl)methyl)-N-hydroxybenzamide |
| 342 | 4-(((3R,5S)-4-(3-(furan-2-yl)benzyl)-3,5-dimethylpiperazin-1- |
| | yl)methyl)-N-hydroxybenzamide |
| 343 | 4-(((3R,5S)-4-(3-(furan-3-yl)benzyl)-3,5-dimethylpiperazin-1- |
| | yl)methyl)-N-hydroxybenzamide |
| 344 | 4-(((3R,5S)-3,5-dimethyl-4-(3-(pyridin-3-yl)benzyl)piperazin-1- |
| | yl)methyl)-N-hydroxybenzamide |
| 345 | 4-(((3R,5S)-3,5-dimethyl-4-(3-(pyridin-4-yl)benzyl)piperazin-1- |
| | yl)methyl)-N-hydroxybenzamide |
| 346 | 4-(((3R,5S)-4-(3-(3,6-dihydro-2H-pyran-4-y1Jbenzyl)-3,5-dimethylpiperazin- |
| | 1-yl)methyl)-N-hydroxybenzamide |
| 349 | 4-(((2S,6R)-2,6-dimethyl-4-(2-(pyridin-3-yl)benzyl)piperazin-1- |
| | yl)methyl)-N-hydroxybenzamide |
| 354 | 4-(((3R,5S)-3,5-dimethyl-4-(oxazol-4-yl)methyl)piperazin-1- |
| | yl)methyl)-N-hydroxybenzamide |
| 356 | 4-(((3R,5S)-3,5-dimethyl-4-(3-(morpholinomethyl)benzyl)piperazin- |
| | 1-yl)methyl)-N-hydroxybenzamide |
| 376 | 4-(((3R,5S)-3,5-dimethyl-4-(4-(morpholinomethyl)benzyl)piperazin- |
| | 1-yl)methyl)-N-hydroxybenzamide |
| 380 | 4-(((3R,5S)-4-(3-((1-(2-fluoro-2-methylpropyl)piperidin-4-yl)methoxy)benzyl)-3,5- |
| | dimethylpiperazin-1-yl)methyl)-N-hydroxybenzamide |
| 382 | 4-(((3R,5S)-3,5-dimethyl-4-(3-(piperidin-1-yl)methyl)benzyl)piperazin-1- |
| | yl)methyl)-N-hydroxybenzamide |
| 383 | 4-(((3R,5S)-4-(3-(((R)-3-fluoropyrrolidin-1-yl)methyl)benzyl)-3,5- |
| | dimethylpiperazin-1-yl)methyl)-N-hydroxybenzamide |
| 384 | 4-(((3R,5S)-4-(3-((3,3-difluoroazetidin-1-yl)methyl)benzyl)-3,5- |
| | dimethylpiperazin-1-yl)methyl)-N-hydroxybenzamide |
| 385 | 4-(((3R,5S)-4-(3-((4-benzylpiperazin-1-yl)methyl)benzyl)-3,5- |
| | dimethylpiperazin-1-yl)methyl)-N-hydroxybenzamide |
| 386 | N-hydroxy-4-(((3R,5S)-4-(3-((4-(2-hydroxy-2-methylpropyl)piperazin-1- |
| | yl)methyl)benzyl)-3,5-dimethylpiperazin-1-yl)methyl)benzamide |
| 387 | 4-(((3R,5S)-4-(3-((4-(2-fluoro-2-methylpropyl)piperazin-1- |
| | yl)methyl)benzyl)-3,5-dimethylpiperazin-1-yl)methyl)-N-hydroxybenzamide |
| 388 | 4-(((3R,5S)-4-(4-((1-(2-fluoro-2-methylpropyl)piperidin-4- |
| | yl)methoxy)benzyl)-3,5-dimethylpiperazin-1-yl)methyl)-N-hydroxybenzamide |
| 403 | 4-(((3R,5S)-3,5-dimethyl-4-(1H-pyrazole-3-carbonyl)piperazin-1- |
| | yl)methyl)-N-hydroxybenzamide |
| 411 | 4-(((3R,5S)-4-(4-(((R)-3-fluoropyrrolidin-1-yl)methyl)benzyl)- |
| | 3,5-dimethylpiperazin-1-yl)methyl)-N-hydroxybenzamide |
| 413 | 4-(((3R,5S)-4-(4-((4-benzylpiperazin-1-yl)methyl)benzyl)- |
| | 3,5-dimethylpiperazin-1-yl)methyl)-N-hydroxybenzamide |
| 430 | N-hydroxy-4-(((3R,5S)-4-(3-((4-isopropylpiperazin-1- |
| | yl)methyl)benzyl)-3,5-dimethylpiperazin-1-yl)methyl)benzamide |
| 431 | 4-(((3R,5S)-4-(3-((4-acetylpiperazin-1-yl)methyl)benzyl)- |
| | 3,5-dimethylpiperazin-1-yl)methyl)-N-hydroxybenzamide |
| 432 | 4-(((3R,5S)-3,5-dimethyl-4-(3-((4-methylpiperazin-1- |
| | yl)methyl)benzoyl)piperazin-1-yl)methyl)-N-hydroxybenzamide |
| 433 | 4-(((3R,5S)-4-(3-((4-ethylpiperazin-1-yl)methyl)benzoyl)- |
| | 3,5-dimethylpiperazin-1-yl)methyl)-N-hydroxybenzamide |
| 434 | N-hydroxy-4-(((3R,5S)-4-(3-((4-isopropylpiperazin-1- |
| | yl)methyl)benzoyl)-3,5-dimethylpiperazin-1-yl)methyl)benzamide |
| 435 | 4-(((3R,5S)-4-(3-((4-acetylpiperazin-1-yl)methyl)benzoyl)- |
| | 3,5-dimethylpiperazin-1-yl)methyl)-N-hydroxybenzamide |
| 439 | 4-(((3R,5S)-3,5-dimethyl-4-(3-(morpholinomethyl)benzoyl)piperazin- |
| | 1-yl)methyl)-N-hydroxybenzamide |
| 440 | 4-(((3R,5S)-4-(3-(((1-(diethylamino)propan-2-yl)amino)methyl)benzoyl)- |
| | 3,5-dimethylpiperazin-1-yl)methyl)-N-hydroxybenzamide |
| 441 | 4-(((3R,5S)-4-(3-(((1-(benzyl(methyl)amino)propan-2-yl)amino)methyl)benzoyl)- |
| | 3,5-dimethylpiperazin-1-yl)methyl)-N-hydroxybenzamide |
| 442 | N-hydroxy-4-(((3R,5S)-4-(3-((4-isopentylpiperazin-1-yl)methyl)benzoyl)- |
| | 3,5-dimethylpiperazin-1-yl)methyl)benzamide |
| 443 | 4-(((3R,5S)-3,5-dimethyl-4-(3-((4-(2,2,2-trifluoroethyl)piperazin-1- |
| | yl)methyl)benzoyl)piperazin-1-yl)methyl)-N-hydroxybenzamide |
| 444 | 4-(((3R,5S)-3,5-dimethyl-4-(3-((4-(methylsulfonyl)piperazin-1- |
| | yl)methyl)benzoyl)piperazin-1-yl)methyl)-N-hydroxybenzamide |
| 452 | 4-(((3R,5S)-3,5-dimethyl-4-(3-((4-(methylsulfonyl)piperazin-1- |
| | yl)methyl)benzyl)piperazin-1-yl)methyl)-N-hydroxybenzamide |
| 453 | 4-(((3R,5S)-3,5-dimethyl-4-(3-((4-(2,2,2-trifluoroethyl)piperazin-1- |
| | yl)methyl)benzyl)piperazin-1-yl)methyl)-N-hydroxybenzamide |
| 454 | N-hydroxy-4-(((3R,5S)-4-(3-((4-isopentylpiperazin-1-yl)methyl)benzyl)-3,5- |
| | dimethylpiperazin-1-yl)methyl)benzamide |
| 455 | 4-(((3R,5S)-4-(3-(((1-(diethylamino)propan-2-yl)amino)methyl)benzyl)- |
| | 3,5-dimethylpiperazin-1-yl)methyl)-N-hydroxybenzamide |
| 456 | 4-(((3R,5S)-4-(3-(((1-(benzyl(methyl)amino)propan-2-yl)amino)methyl)benzyl)- |
| | 3,5-dimethylpiperazin-1-yl)methyl)-N-hydroxybenzamide |
| 467 | 4-(((3R,5S)-3,5-dimethyl-4-(4-(piperidin-1-yl)methyl)benzyl)piperazin-1- |
| | yl)methyl)-N-hydroxybenzamide |
| 468 | 4-(((3R,5S)-3,5-dimethyl-4-(4-((4-methylpiperazin-1-yl)methyl)benzyl)piperazin- |
| | 1-yl)methyl)-N-hydroxybenzamide |
| 481 | 4-(((3R,5S)-3,5-dimethyl-4-(2-(1-(methylsulfonyl)-1,2,3,6-tetrahydropyridin- |
| | 4-yl)benzyl)piperazin-1-yl)methyl)-N-hydroxybenzamide |
| 482 | 4-(((3R,5S)-4-(2-(1-acetyl-1,2,3,6-tetrahydropyridin-4-yl)benzyl)- |
| | 3,5-dimethylpiperazin-1-yl)methyl)-N-hydroxybenzamide |
| 483 | 4-(((3R,5S)-3,5-dimethyl-4-(2-(1-(2,2,2-trifluoroethyl)-1,2,3,6-tetrahydropyridin- |
| | 4-yl)benzyl)piperazin-1-yl)methyl)-N-hydroxybenzamide |
| 484 | N-hydroxy-4-(((3R,5S)-4-(2-(1-isopropyl-1,2,3,6-tetrahydropyridin-4-yl)benzyl)- |
| | 3,5-dimethylpiperazin-1-yl)methyl)benzamide |
| 485 | 4-(((3R,5S)-4-(2-(1-ethyl-1,2,3,6-tetrahydropyridin-4-yl)benzyl)-3,5- |
| | dimethylpiperazin-1-yl)methyl)-N-hydroxybenzamide |
| 486 | 4-(((3R,5S)-4-(4-((4-acetylpiperazin-1-yl)methyl)benzyl)-3,5- |
| | dimethylpiperazin-1-yl)methyl)-N-hydroxybenzamide (hydrochloride salt) |
| 569 | 5-(((3R,5S)-4-benzyl-3,5-dimethylpiperazin-1-yl)methyl)- |
| | N-hydroxy-1H-indo1e-2-carboxamide |
| 696 | 4-(((3R,5S)-3,5-dimethyl-4-(3-(morpholinomethyl)benzyl)piperazin-1- |
| | yl)methyl)-3-fluoro-N-hydroxybenzamide |
| 813 | 4-(((3R,5S)-4-benzyl-3,5-dimethylpiperazin-1-yl)methyl)- |
| | 3-fluoro-N-hydroxybenzamide |
| 823 | 2-(4-((3R,5S)-4-benzyl-3,5-dimethylpiperazine-1- |
| | carbonyl)phenyl)-N-hydroxyacetamide |

7. The pharmaceutical composition according to claim 6, wherein the compound represented by Formula I is a compound described in the following table:

| No. | Name of Compound |
|---|---|
| 082 | 4-(((3R,5S)-4-acetyl-3,5-dimethylpiperazin-1-yl)methyl)-N- |
| | hydroxybenzamide |
| 083 | 4-(((3R,5S)-4-benzoyl-3,5-dimethylpiperazin-1-yl)methyl)-N- |
| | hydroxybenzamide |
| 084 | 4-(((3R,5S)-4-benzyl-3,5-dimethylpiperazin-1-yl)methyl)-N- |
| | hydroxybenzamide |
| 098 | (2S,6R)-4-(4-(hydroxycarbamoyl)benzyl)-N-isopropyl-2,6- |
| | dimethylpiperazine-1-carboxamide |
| 100 | 4-(((3R,5S)-3,5-dimethyl-4-(phenylsulfonyl)piperazin-1- |
| | yl)methyl)-N-hydroxybenzamide |
| 120 | 4-(((3R,5S)-4-(2-chlorobenzoyl)-3,5-dimethylpiperazin-1- |
| | yl)methyl)-N-hydroxybenzamide |
| 121 | 4-(((3R,5S)-4-(3-chlorobenzoyl)-3,5-dimethylpiperazin-1- |
| | yl)methyl)-N-hydroxybenzamide |
| 122 | 4-(((3R,5S)-4-(4-chlorobenzoyl)-3,5-dimethylpiperazin-1- |
| | yl)methyl)-N-hydroxybenzamide |
| 123 | 4-(((3R,5S)-3,5-dimethyl-4-picolinoylpiperazin-1- |
| | yl)methyl)-N-hydroxybenzamide |
| 125 | 4-(((3R,5S)-4-(furan-2-carbonyl)-3,5-dimethylpiperazin-1- |
| | yl)methyl)-N-hydroxybenzamide |
| 126 | 4-(((3R,5S)-3,5-dimethyl-4-(thiophene-2-carbonyl)piperazin- |
| | 1-yl)methyl)-N-hydroxybenzamide |
| 127 | 4-(((3R,5S)-4-(2-chlorobenzyl)-3,5-dimethylpiperazin-1- |
| | yl)methyl)-N-hydroxybenzamide |
| 128 | 4-(((3R,5S)-4-(3-chlorobenzyl)-3,5-dimethylpiperazin-1- |
| | yl)methyl)-N-hydroxybenzamide |
| 145 | 4-(((3R,5S)-4-(furan-2-yl)methyl)-3,5-dimethylpiperazin-1- |
| | yl)methyl)-N-hydroxybenzamide |
| 146 | 4-(((3R,5S)-3,5-dimethyl-4-(2-phenylacetyl)piperazin-1- |
| | yl)methyl)-N-hydroxybenzamide |
| 148 | 4-(((3R,5S)-3,5-dimethyl-4-propylpiperazin-1-yl)methyl)-N- |
| | hydroxybenzamide |
| 149 | 4-(((3R,5S)-3,5-dimethyl-4-(2,2,2-trifluoroethyl)piperazin-1- |
| | yl)methyl)-N-hydroxybenzamide |
| 159 | (2S,6R)-4-(4-(hydroxycarbamoyl)benzyl)-2,6-dimethyl-N- |
| | phenylpiperazine-1-carboxamide |
| 160 | (2S,6R)-4-(4-(hydroxycarbamoyl)benzyl)-2,6-dimethyl-N-(o-tolyl) |
| | piperazine-1-carboxamide |
| 161 | (2S,6R)-N-benzyl-4-(4-(hydroxycarbamoyl)benzyl)-2,6- |
| | dimethylpiperazine-1-carboxamide |
| 171 | 4-(((3R,5S)-4-benzyl-3,5-dimethylpiperazin-1-yl)methyl)-2- |
| | fluoro-N-hydroxybenzamide |
| 174 | 4-(((2S,6R)-4-benzyl-2,6-dimethylpiperazin-1-yl)methyl)-2- |
| | fluoro-N-hydroxybenzamide |
| 177 | phenyl (2S,6R)-4-(4-(hydroxycarbamoyl)benzyl)-2,6- |
| | dimethylpiperazine-1-carboxylate |
| 194 | 6-(((3R,5S)-4-benzyl-3,5-dimethylpiperazin-1-yl)methyl)- |
| | N-hydroxynicotinamide |
| 211 | 4-(((3R,5S)-4-(2-fluorobenzyl)-3,5-dimethylpiperazin-1- |
| | yl)methyl)-N-hydroxybenzamide |
| 249 | 4-(((3R,5R)-4-(furan-2-carbonyl)-3,5-dimethylpiperazin-1- |
| | yl)methyl)-N-hydroxybenzamide |
| 255 | 4-(((3R,5S)-4-(3-(1H-pyrrol-1-yl)benzyl)-3,5-dimethylpiperazin-1- |
| | yl)methyl)-N-hydroxybenzamide |
| 283 | 4-(((3R,5S)-4-(2-(furan-2-yl)benzoyl)-3,5-dimethylpiperazin-1- |
| | yl)ethyl)-N-hydroxybenzamide |
| 305 | 4-(((2S,6R)-2,6-dimethyl-4-(2-(2-methyl-1H-imidazo1-1- |
| | yl)benzyl)piperazin-1-yl)methyl)-N-hydroxybenzamide |
| 326 | 4-(((3R,5S)-4-(2-(3,6-dihydro-2H-pyran-4-yl)benzoyl)-3,5- |
| | dimethylpiperazin-1-yl)methyl)-N-hydroxybenzamide |
| 328 | 4-(((3R,5S)-4-(2-(furan-3-yl)benzyl)-3,5-dimethylpiperazin- |
| | 1-yl)methyl)-N-hydroxybenzamide |
| 329 | 4-(((3R,5S)-3,5-dimethyl-4-(2-(pyridin-4-yl)benzyl)piperazin-1- |
| | yl)methyl)-N-hydroxybenzamide |
| 330 | 4-(((3R,5S)-3,5-dimethyl-4-(2-(pyridin-3-yl)benzyl)piperazin-1- |
| | yl)methyl)-N-hydroxybenzamide |
| 332 | 4-(((3R,5S)-4-(2-(3,6-dihydro-2H-pyran-4-yl)benzyl)-3,5- |
| | dimethylpiperazin-1-yl)methyl)-N-hydroxybenzamide |
| 342 | 4-(((3R,5S)-4-(3-(furan-2-yl)benzyl)-3,5-dimethylpiperazin-1- |
| | yl)methyl)-N-hydroxybenzamide |
| 343 | 4-(((3R,5S)-4-(3-(furan-3-yl)benzyl)-3,5-dimethylpiperazin-1- |
| | yl)methyl)-N-hydroxybenzamide |
| 344 | 4-(((3R,5S)-3,5-dimethyl-4-(3-(pyridin-3-yl)benzyl)piperazin-1- |
| | yl)methyl)-N-hydroxybenzamide |
| 345 | 4-(((3R,5S)-3,5-dimethyl-4-(3-(pyridin-4-yl)benzyl)piperazin-1- |
| | yl)methyl)-N-hydroxybenzamide |
| 346 | 4-(((3R,5S)-4-(3-(3,6-dihydro-2H-pyran-4-yl)benzyl)-3,5- |
| | dimethylpiperazin-1-yl)methyl)-N-hydroxybenzamide |
| 349 | 4-(((2S,6R)-2,6-dimethyl-4-(2-(pyridin-3-yl)benzyl)piperazin-1- |
| | yl)methyl)-N-hydroxybenzamide |
| 354 | 4-(((3R,5S)-3,5-dimethyl-4-(oxazol-4-ylmethyl)piperazin-1- |
| | yl)methyl)-N-hydroxybenzamide |
| 356 | 4-(((3R,5S)-3,5-dimethyl-4-(3-(morpholinomethyl)benzyl)piperazin-1- |
| | yl)methyl)-N-hydroxybenzamide |
| 376 | 4-(((3R,5S)-3,5-dimethyl-4-(4-(morpholinomethyl)benzyl)piperazin-1- |
| | yl)methyl)-N-hydroxybenzamide |
| 382 | 4-(((3R,5S)-3,5-dimethyl-4-(3-(piperidin-1-yl)methyl)benzyl)piperazin-1- |
| | yl)methyl)-N-hydroxybenzamide |
| 383 | 4-(((3R,5S)-4-(3-(((R)-3-fluoropyrrolidin-1-yl)methyl)benzyl)-3,5- |
| | dimethylpiperazin-1-yl)methyl)-N-hydroxybenzamide |
| 387 | 4-(((3R,5S)-4-(3-((4-(2-fluoro-2-methylpropyl)piperazin-1-yl)methyl)benzyl)- |
| | 3,5-dimethylpiperazin-1-yl)methyl)-N-hydroxybenzamide |
| 388 | 4-(((3R,5S)-4-(4-((1-(2-fluoro-2-methylpropyl)piperidin-4-yl)methoxy)benzyl)- |
| | 3,5-dimethylpiperazin-1-yl)methyl)-N-hydroxybenzamide |
| 411 | 4-(((3R,5S)-4-(4-(((R)-3-fluoropyrrolidin-1-yl)methyl)benzyl)-3,5- |
| | dimethylpiperazin-1-yl)methyl)-N-hydroxybenzamide |
| 413 | 4-(((3R,5S)-4-(4-((4-benzylpiperazin-1-yl)methyl)benzyl)-3,5- |
| | dimethylpiperazin-1-yl)methyl)-N-hydroxybenzamide |
| 431 | 4-(((3R,5S)-4-(3-((4-acetylpiperazin-1-yl)methyl)benzyl)-3,5- |
| | dimethylpiperazin-1-yl)methyl)-N-hydroxybenzamide |
| 439 | 4-(((3R,5S)-3,5-dimethyl-4-(3-(morpholinomethyl)benzoyl)piperazin-1- |
| | yl)methyl)-N-hydroxybenzamide |
| 441 | 4-(((3R,5S)-4-(3-(((1-(benzyl(methyl)amino)propan-2-yl)amino)methyl)benzoyl)- |
| | 3,5-dimethylpiperazin-1-yl)methyl)-N-hydroxybenzamide |
| 444 | 4-(((3R,5S)-3,5-dimethyl-4-(3-((4-(methylsulfonyl)piperazin-1- |
| | yl)methyl)benzoyl)piperazin-1-yl)methyl)-N-hydroxybenzamide |
| 452 | 4-(((3R,5S)-3,5-dimethyl-4-(3-((4-(methylsulfonyl)piperazin-1- |
| | yl)methyl)benzyl)piperazin-1-yl)methyl)-N-hydroxybenzamide |
| 453 | 4-(((3R,5S)-3,5-dimethyl-4-(3-((4-(2,2,2-trifluoroethyl)piperazin-1- |
| | yl)methyl)benzyl)piperazin-1-yl)methyl)-N-hydroxybenzamide |
| 454 | N-hydroxy-4-(((3R,5S)-4-(3-((4-isopentylpiperazin-1- |
| | yl)methyl)benzyl)-3,5-dimethylpiperazin-1-yl)methyl)benzamide |
| 467 | 4-(((3R,5S)-3,5-dimethyl-4-(4-(piperidin-1-yl)methyl)benzyl)piperazin-1- |
| | yl)methyl)-N-hydroxybenzamide |
| 468 | 4-(((3R,5S)-3,5-dimethyl-4-(4-((4-methylpiperazin-1-yl)methyl)benzyl)piperazin-1- |
| | yl)methyl)-N-hydroxybenzamide |
| 481 | 4-(((3R,5S)-3,5-dimethyl-4-(2-(1-(methylsulfonyl)-1,2,3,6-tetrahydropyridin- |
| | 4-yl)benzyl)piperazin-1-yl)methyl)-N-hydroxybenzamide |
| 482 | 4-(((3R,5S)-4-(2-(1-acetyl-1,2,3,6-tetrahydropyridin-4-yl)benzyl)- |
| | 3,5-dimethylpiperazin-1-yl)methyl)-N-hydroxybenzamide |
| 483 | 4-(((3R,5S)-3,5-dimethyl-4-(2-(1-(2,2,2-trifluoroethyl)-1,2,3,6- |
| | tetrahydropyridin-4-yl)benzyl)piperazin-1-yl)methyl)-N-hydroxybenzamide |
| 484 | N-hydroxy-4-(((3R,5S)-4-(2-(1-isopropyl-1,2,3,6-tetrahydropyridin- |
| | 4-yl)benzyl)-3,5-dimethylpiperazin-1-yl)methyl)benzamide |
| 485 | 4-(((3R,5S)-4-(2-(1-ethyl-1,2,3,6-tetrahydropyridin-4-yl)benzyl)- |
| | 3,5-dimethylpiperazin-1-yl)methyl)-N-hydroxybenzamide |
| 696 | 4-(((3R,5S)-3,5-dimethyl-4-(3-(morpholinomethyl)benzyl)piperazin-1- |
| | yl)methyl)-3-fluoro-N-hydroxybenzamide |
| 813 | 4-(((3R,5S)-4-benzyl-3,5-dimethylpiperazin-1-yl)methyl)-3-fluoro-N-hydroxybenzamide |

8. The pharmaceutical composition according to claim 1, wherein the compound represented by Formula I, isomers thereof or pharmaceutically acceptable salts thereof inhibit histone deacetylase 6 (HDAC6).

9. Use of the compound represented by Formula I according to claim 1, isomers thereof or pharmaceutically acceptable salts thereof in preparation of a medicament for preventing or treating Charcot-Marie-Tooth (CMT) disease.

10. A method for preventing or treating Charcot-Marie-Tooth (CMT) disease, comprising a step of administering a therapeutically effective amount of the compound represented by Formula I according to claim 1, isomers thereof or pharmaceutically acceptable salts thereof into a subject.

11. Use of the compound represented by Formula I according to claim 1, isomers thereof or pharmaceutically acceptable salts thereof for preventing or treating Charcot-Marie-Tooth (CMT) diseases.
